(19) United States
(12) Patent Application Publication
(10) Pub. No.: US 2026/0034107 A1
(43) Pub. Date: Feb. 5, 2026

(54) SUBSTITUTED FUSED IMIDAZOLE DERIVATIVES AND METHODS OF TREATING SICKLE CELL DISEASE AND RELATED COMPLICATIONS

(71) Applicant: vTv Therapeutics LLC, High Point, NC (US)

(72) Inventor: Otis Clinton ATTUCKS, Winston-Salem, NC (US)

(21) Appl. No.: 19/359,407

(22) Filed: Oct. 15, 2025

Related U.S. Application Data
(63) Continuation of application No. 17/374,407, filed on Jul. 13, 2021.
(63) Continuation of application No. PCT/US2020/013616, filed on Jan. 15, 2020.
(60) Provisional application No. 62/794,293, filed on Jan. 18, 2019.

(51) Int. Cl. A61K 31/437; A61K 31/428; A61K 45/06

(52) U.S. Cl. CPC A61K 31/437; A61K 31/428; A61K 45/06

(57) ABSTRACT

The present invention provides methods of treating sickle cell disease and related complications using compounds of Formula (I) and pharmaceutical compositions thereof either alone or in combination other active agents.

### FIELD OF THE INVENTION

The present invention provides methods of treating sickle cell disease and related complications using compounds of Formula (I) and pharmaceutical compositions thereof either alone or in combination with other active agents. The present invention also provides compounds and pharmaceutical compositions.

### BACKGROUND OF THE INVENTION

Sickle cell disease (SCD) is a life-threatening monogenic disorder. SCD is a severe hemoglobinopathy that produces multisystem complications due to the expression of abnormal sickle hemoglobin (HbS). The most common type of SCD is sickle cell anemia (SCA) (also referred to as HbSS or SS disease or hemoglobin S) in which there is homozygosity for the mutation that causes HbS. The more rare types of SCD in which there is heterozygosity (one copy of the mutation that causes HbS and one copy for another abnormal hemoglobin allele) for the mutation include sickle-hemoglobin C (HbSC), sickle ß⁺ thalassemia (HbS/ß+) and sickle ß0 thalassemia (HbS/ß⁰).

Sickle cell disease (SCD) arise from a point mutation that causes erythrocyte deformation or sickle-shaped erythrocytes. Sickled-shaped erythrocytes are associated with clinical manifestations of SCD, such as anemia, recurrent painful vaso-occlusive episodes, infections, acute chest syndrome, pulmonary hypertension, stroke, priapism, osteonecrosis, renal insufficiency, leg ulcers, retinopathies, and cardiac disease.

SCD arises from a single point mutation (GAG>GTG) in codon 6 of the HBB globin gene. The deoxygenated venous circulation causes a process of self-assembly (polymerization) that generates the sickled hemoglobin molecule (HbS) and damages the membrane and cytoskeleton of the erythrocyte. The HbS repetitively enter into sickling and unsickling cycles incrementally increasing the damage to the erythrocyte membrane (Ischemia-reperfusion (IR) injury) resulting in irreversibly sickle-shaped erythrocytes. As a consequence, these rigid blood cells are unable to deform as they pass through narrow capillaries, leading to vessel occlusion and ischemia. The actual anemia of the illness is caused by hemolysis, the destruction of the red cells, caused by their misshapes.

C-reactive protein (CRP) and the markers of oxidative stress are significantly increased following IR injury. The ensuing oxidative stress contributes to hemolysis, inactivation of nitric oxide (NO), and erythrocyte, leukocyte and platelet adhesive properties.

The sickled-shaped erythrocytes together with endothelial cells, activated leukocytes, platelets and plasma proteins participate in the multistep vaso-occlusion process.

Heme oxygenase-1 (HO-1) and interleukin 10 (IL-10) are characteristically found to be increased in SCD patients in an attempt to counteract the induced inflammation. HO-1 breaks down heme released during hemolysis thereby limiting oxidative stress and inflammation, while IL-10 limits the production of the pro-inflammatory cytokines.

Sickled erythrocytes stimulates leukocyte recruitment: ensuing the inflammatory stimulus, leukocytes are recruited to the activated endothelium of the venous circulation where it forms adhesive interactions with the activated endothelium and sickled erythrocytes, leading to a reduced blood flow and eventually vaso-occlusion.

SCD platelets show increased surface expressions of selectin P (SELP), activated α_{IIIb} ß₃ (GPIIbIIIa) and higher concentrations of the platelet activation markers. In healthy individuals, platelet adhesion is inhibited by the antithrombotic factor NO, while SCD platelet adhesion is stimulated by the activated endothelium. Platelets and sickled erythrocytes have been demonstrated to aggregate via the formation of thrombospondin bridges thereby contributing to vaso-occlusion.

Hydroxyurea (HU) is an approved treatment to modify the disease process of SCD. HU counteracts the pathophysiology of SCD by increasing the production of fetal hemoglobin (HbF)-containing erythrocytes and indirectly altering gene expression and proteins associated with the pathophysiology of SCD. The increased concentration of HbF-containing erythrocytes dilutes the concentration of sickled erythrocytes, may thereby sequentially trigger decreased hemolysis, increased NO bioavailability and decreased endothelium activation. However, HU has been demonstrated to reduce leukocyte counts in patients on therapy. Although HU improved clinical symptoms by reducing pain and vaso-occlusive crises, acute chest syndrome, transfusion requirements, and hospitalization, SCD patients treated with HU have demonstrated side effects such as inducing DNA damage, reducing sperm counts and producing iron nitrosyl Hb.

Thus, there is a need in the art for new, improved, and/or complimentary SCD therapies.

### SUMMARY OF THE INVENTION

PCT Publication No. WO 2011/103018 (“WO '018”) describes substituted fused imidazole derivatives that upregulate expression of HMOX1 in vitro. PCT Publication No. WO 2012/094580 (“WO '580”) describes various compounds that modulate cellular oxidative stress including fused imidazole derivatives having a structure similar to or the same as compounds disclosed in WO '018.

The present invention is directed to methods and compositions associated with treatment of one or more blood disorders. Although in particular embodiments the blood disorder is SCD, in specific embodiments, one or more other blood disorders may be treated with the present invention: a bleeding disorder (including clotting disorders, hypercoagulability, hemophilia, or von Willebrand disease, for example), platelet disorder (essential or primary thrombocythemia or thrombocytopenia, for example), and/or hemophilia or anemia may be treated, for example. In particular embodiments of the invention, there are methods and compositions for treatment and/or prevention of sickle cell disease (which may be referred to as sickle-cell anemia (or anemia; SCA) or drepanocytosis).

Mammalian and/or non-human mammals or cell lines may be used as sickle cell models. The individual treated with methods and/or compositions of the invention may be experiencing vaso-occlusive crisis, acute chest crisis, painful chest syndrome that may or may not require hospitalization, in specific cases. In specific embodiments, the individual may be experiencing or may experience negative side effects of a drug, such as a drug that directly or indirectly results in increased coagulation and/or increased inflammation; in specific embodiments, the drug is HU.

In certain embodiments of the invention, a compound of the invention is administered alone. In other embodiment, a compound of the invention is administered with one or more other drugs (some of which may or may not induce HbF production) for the treatment of SCD. For example, a compound of the invention may be administered in combination with HU for the treatment of SCD. In another example, a compound of the invention may be administered in combination with an Nrf2 activator, such as a fumarate ester (MMF or DMF) and bardoxolone methyl.

The individual treated may be known to have SCD, is suspected of or at risk for having SCD. In embodiments of the invention, an individual is diagnosed with sickle cell disease prior to receiving the inventive treatment.

The present invention is also directed to compounds of Formula (I) and pharmaceutically acceptable salts thereof and to pharmaceutical compositions comprising Formula (I) and pharmaceutically acceptable salts thereof, and methods of making thereof.

### BRIEF DESCRIPTION OF THE DRAWINGS

FIGS. 1A-1D show the relative cell growth and percent cell viability of KU812 cells in the presence of various concentrations (0, 0.5, 2.5, 5, 10, and 20 μM) of Compounds 73, 134, 473, and 236, respectively. Data are presented mean±SD (n=3). *, p<0.05

FIG. 1E comprises Western Blots showing the level of induction of HbF following treatment of KU812 cells with various concentrations (0, 0.5, 2.5, 5, 10, and 20 μM) of Compounds 73, 134, 473, and 236. Hydroxyurea (HU) and hemin were used as HbF induction positive controls, and β-actin was used as a protein loading control.

FIG. 2 shows HbF protein expression levels of KU812 cells obtained by FACs and analyzed as the mean concentration of HbF per cell measured by mean fluorescence intensity (MFI).

FIG. 3A comprises Western Blots showing the level of induction of HbF and HbS when sickle erythroid progenitor cells were treated with Compound 473 (0.5 and 2.5 μM) for 48 hours. Hydroxyurea (HU) and hemin were used as HbF induction positive controls, and ß-actin was used as a protein loading control.

FIG. 3B shows the percent of HbF positive cells (F-cells) when sickle erythroid progenitor cells were treated with Compound 473 (0.5 and 2.5 μM) for 48 hours and analyzed by flow cytometry. Hydroxyurea (HU) and hemin were used as HbF induction positive controls.

FIG. 4A contains images of sickle erythroid progenitor cells after culturing for 10 days, treating with Compound 473 for 48 hours at concentrations of 0.5 μM and 2.5 μM or with hemin (about 50 μM) or with hydroxyurea (HU) (about 100 μM), and then subjecting the cells to hypoxia conditions (1% 02 and 5% CO₂).

FIG. 4B shows the percent of sickled cells when sickle erythroid progenitor cells were cultured for 10 days and then treated with Compound 473 for 48 hours at concentrations of 0.5 μM and 2.5 μM or with hemin (about 50 μM) or with hydroxyurea (HU) (about 100 μM), and then subjected to hypoxia conditions (1% O₂ and 5% CO₂).

### DETAILED DESCRIPTION OF INVENTION

### I. Definitions

The following definitions are intended to clarify the terms defined. If a particular term used herein is not specifically defined, the term should not be considered to be indefinite. Rather, such undefined terms are to be construed in accordance with their plain and ordinary meaning to a person of ordinary skill in the field(s) of art to which the invention is directed.

As used herein the term “alkyl” refers to a straight or branched chain saturated hydrocarbon having one to ten carbon atoms, which may be optionally substituted, as herein further described, with multiple degrees of substitution being allowed. Examples of “alkyl” as used herein include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl.

The number carbon atoms in an alkyl group is represented by the phrase “C_{x-y} alkyl,” which refers to an alkyl group, as herein defined, containing from x to y, inclusive, carbon atoms. Thus, C₁₋₆ alkyl represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl.

As used herein, the term “alkylene” refers to a straight or branched chain divalent saturated hydrocarbon radical having from one to ten carbon atoms, which may be optionally substituted as herein further described, with multiple degrees of substitution being allowed. Examples of “alkylene” as used herein include, but are not limited to, methylene, ethylene, n-propylene, 1-methylethylene, 2-methylethylene, dimethylmethylene, n-butylene, 1-methyl-n-propylene, and 2-methyl-n-propylene.

The number of carbon atoms in an alkylene group is represented by the phrase “C_{x-y} alkylene,” which refers to an alkylene group, as herein defined, containing from x to y, inclusive, carbon atoms. Similar terminology will apply for other terms and ranges as well. Thus, C₁₋₄ alkylene represents an alkylene chain having from 1 to 4 carbons atoms, and, for example, includes, but is not limited to, methylene, ethylene, n-propylene, 1-methylethylene, 2-methylethylene, dimethylmethylene, n-butylene, 1-methyl-n-propylene, and 2-methyl-n-propylene.

As used herein, the term “cycloalkyl” refers to a saturated, three-to ten-membered, cyclic hydrocarbon ring, which may be optionally substituted as herein further described, with multiple degrees of substitution being allowed. Such “cycloalkyl” groups are monocyclic, bicyclic, or tricyclic. Examples of “cycloalkyl” groups as used herein include, but are not limited to, cyclopropyl, cyclobutyl, cyclopentyl, cyclohexyl, cycloheptyl, norbornyl, and adamantyl.

The number of carbon atoms in a cycloalkyl group will be represented by the phrase “C_{x-y} cycloalkyl,” which refers to a cycloalkyl group, as herein defined, containing from x to y, inclusive, carbon atoms. Similar terminology will apply for other terms and ranges as well. Thus, C₃₋₁₀ cycloalkyl represents a cycloalkyl group having from 3 to 10 carbons as described above, and for example, includes, but is not limited to, cyclopropyl, cyclobutyl, cyclopentyl, cyclohexyl, cycloheptyl, norbornyl, and adamantyl.

As used herein, the term “heterocycle” or “heterocyclyl” refers to an optionally substituted mono- or polycyclic saturated ring system containing one or more heteroatoms. Such “heterocycle” or “heterocyclyl” groups may be optionally substituted as herein further described, with multiple degrees of substitution being allowed. The term “heterocycle” or “heterocyclyl,” as used herein, does not include ring systems that contain one or more aromatic rings. Examples of heteroatoms include nitrogen, oxygen, or sulfur atoms, including N-oxides, sulfur oxides, and sulfur dioxides. Typically, the ring is three-to twelve-membered. Such rings may be optionally fused to one or more of another heterocyclic ring(s) or cycloalkyl ring(s). Examples of “heterocyclic” groups, as used herein include, but are not limited to, tetrahydrofuran, tetrahydropyran, 1,4-dioxane, 1,3-dioxane, piperidine, pyrrolidine, morpholine, tetrahydrothiopyran, and tetrahydrothiophene, where attachment can occur at any point on said rings, as long as attachment is chemically feasible. Thus, for example, “morpholine” refers to morpholin-2-yl, morpholin-3-yl, and morpholin-4-yl.

As used herein, when “heterocycle” or “heterocyclyl” is recited as a possible substituent, the “heterocycle” or “heterocyclyl” group can attach through either a carbon atom or any heteroatom, to the extent that attachment at that point is chemically feasible. For example, “heterocyclyl” would include pyrrolidin-1-yl, pyrrolidin-2-yl, and pyrrolidin-3-yl. When “heterocycle” or “heterocyclyl” groups contain a nitrogen atom in the ring, attachment through the nitrogen atom can alternatively be indicated by using an “-ino” suffix with the ring name. For example, pyrrolidino refers to pyrrolidin-1-yl.

As used herein the term “halogen” refers to fluorine, chlorine, bromine, or iodine.

As used herein, the term “oxo” refers to a >C═O substituent. When an oxo substituent occurs on an otherwise saturated group, such as with an oxo-substituted cycloalkyl group (e.g., 3-oxo-cyclobutyl), the substituted group is still intended to be a saturated group.

As used herein, the term “heteroaryl” refers to a five-to fourteen-membered optionally substituted mono- or polycyclic ring system, which contains at least one aromatic ring and also contains one or more heteroatoms. Such “heteroaryl” groups may be optionally substituted as herein further described, with multiple degrees of substitution being allowed. In a polycyclic “heteroaryl” group that contains at least one aromatic ring and at least one non-aromatic ring, the aromatic ring(s) need not contain a heteroatom. Thus, for example, “heteroaryl,” as used herein, would include indolinyl. Further, the point of attachment may be to any ring within the ring system without regard to whether the ring containing the attachment point is aromatic or contains a heteroatom. Thus, for example, “heteroaryl,” as used herein, would include indolin-1-yl, indolin-3-yl, and indolin-5-yl. Examples of heteroatoms include nitrogen, oxygen, or sulfur atoms, including N-oxides, sulfur oxides, and sulfur dioxides, where feasible. Examples of “heteroaryl” groups, as used herein include, but are not limited to, furyl, thiophenyl, pyrrolyl, oxazolyl, thiazolyl, imidazolyl, isoxazolyl, isothiazolyl, 1,2,4-triazolyl, pyrazolyl, pyridinyl, pyridazinyl, pyrimidinyl, indolyl, isoindolyl, benzo[b]thiophenyl, benzimidazolyl, benzothiazolyl, pteridinyl, and phenazinyl, where attachment can occur at any point on said rings, as long as attachment is chemically feasible. Thus, for example, “thiazolyl” refers to thiazol-2-yl, thiazol-4-yl, and thiaz-5-yl.

As used herein, when “heteroaryl” is recited as a possible substituent, the “heteroaryl” group can attach through either a carbon atom or any heteroatom, to the extent that attachment at that point is chemically feasible.

As used herein, the term “heterocyclylene” refers to an optionally substituted bivalent heterocyclyl group (as defined above). The points of attachment may be to the same ring atom or to different ring atoms, as long as attachment is chemically feasible. The two points of attachment can each independently be to either a carbon atom or a heteroatom, as long as attachment is chemically feasible. Examples include, but are not limited to,

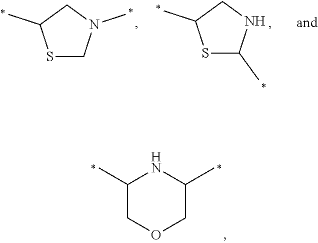

where the asterisks indicate points of attachment.

As used herein, the term “heteroarylene” refers to an optionally substituted bivalent heteroaryl group (as defined above). The points of attachment may be to the same ring atom or to different ring atoms, as long as attachment is chemically feasible. The two points of attachment can each independently be to either a carbon atom or a heteroatom, as long as attachment is chemically feasible. Examples include, but are not limited to,

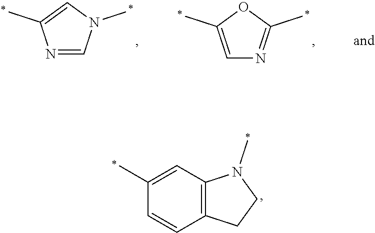

where the asterisks indicate points of attachment.

Various other chemical terms or abbreviations have their standard meaning to the skilled artisan. For example: “hydroxyl” refers to —OH; “methoxy” refers to —OCH₃; “cyano” refers to —CN; “amino” refers to —NH₂; “methylamino” refers to —NHCH₃; “sulfonyl” refers to —SO₂—; “carbonyl” refers to —C(O)—; “carboxy” or “carboxyl” refer to —CO₂H, and the like. Further, when a name recited multiple moieties, e.g., “methylaminocarbonyl-methyl”, an earlier-recited moiety is further from the point of attachment than any later-recited moieties. Thus, a term such as “methylaminocarbonylmethyl” refers to —CH₂—C(O)—NH—CH₃.

As used herein, the term “substituted” refers to substitution of one or more hydrogens of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a subject. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, the various functional groups represented will be understood to have a point of attachment at the functional group having the hyphen or dash (-) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

When any variable occurs more than one time in any one constituent (e.g., R^{d}), or multiple constituents, its definition on each occurrence is independent of its definition on every other occurrence.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

As used herein, the term “optionally” means that the subsequently described event(s) may or may not occur.

As used herein, “administer” or “administering” means to introduce, such as to introduce to a subject a compound or composition. The term is not limited to any specific mode of delivery, and can include, for example, intravenous delivery, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration).

As used herein, “treat” or “treating” or “treatment” can refer to one or more of delaying the progress of a disease or condition, controlling a disease or condition, delaying the onset of a disease or condition, ameliorating one or more symptoms characteristic of a disease or condition, or delaying the recurrence of a disease or condition or characteristic symptoms thereof, depending on the nature of a disease or condition and its characteristic symptoms. “Treat” or “treating” or “treatment” may also refer to inhibiting the disease, either physically, (e.g., stabilization of a discernible symptom), physiologically, (e.g., stabilization of a physical parameter), or both, and to inhibiting at least one physical parameter that may or may not be discernible to the subject. In certain embodiments, “treat” or “treating” or “treatment” refers to delaying the onset of the disease or at least one or more symptoms thereof in a subject which may be exposed to or predisposed to a disease even though that subject does not yet experience or display symptoms of the disease.

As used herein, “subject” may refer any mammal such as, but not limited to, humans. In one embodiment, the subject is a human. In another embodiment, the host is a human who exhibits one or more symptoms characteristic of a disease or condition. The term “subject” does not require one to have any particular status with respect to any hospital, clinic, or research facility (e.g., as an admitted patient, a study participant, or the like). In an embodiment, the subject may be “a subject in need thereof.”

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation.

As used herein, the term “compound of the invention” includes free acids, free bases, and any salts thereof of the compound of Formula (I). Thus, phrases such as “compound of embodiment 1” or “compound of claim 1” refer to any free acids, free bases, and any salts thereof that are encompassed by embodiment 1 or claim 1, respectively.

### II. Methods of Treatment

### A. Treatment of SCD and Related Disorders with Compounds of the Invention

In an embodiment, the present invention provides methods of increasing expression of HbF in cells by contacting certain cells, for example erythroid or retinal pigment epithelial (RPE) cells, with a therapeutically effective amount of a compound of the invention. In other embodiments, the present invention provides methods of increasing expression of HbF in cells by administering a compound of the invention to a subject in need thereof. In embodiment, the expression of HbF is increased such that HbF is greater than or equal to 1%, 2%, 3%, 4%, 5%, 6%, 7%, 8%, 9%, 10%, 12%, 14%, 16%, 18%, 20%, 21%, 22%, 23%, 24%, 25%, 26%, 27%, 28%, 29%, 30%, 35%, 40%, 45%, 50%, 55%, 60%, 65%, 70%, 80%, or 90% of the total hemoglobin in a subject or in a sample taken from a subject. In embodiment, the expression of HbF is increased such that HbF is increased by at least 1%, 2%, 3%, 4%, 5%, 6%, 7%, 8%, 9%, 10%, 12%, 14%, 16%, 18%, 20%, percentage point of the total hemoglobin in a subject or in a sample taken from a subject relative to a baseline sample taken prior to treatment of the subject. In another embodiment where the subject is a human less than 19 years of age, the expression of HbF is increased such that HbF is greater than or equal to 1%, 2%, 3%, 4%, 5%, 6%, 7%, 8%, 9%, 10%, 12%, 14%, 16%, 18%, 20%, 21%, 22%, 23%, 24%, 25%, 26%, 27%, 28%, 29%, 30%, 35%, 40%, 45%, 50%, 55%, 60%, 65%, 70%, 80%, or 90% of the total hemoglobin in a subject or in a sample taken from a subject. The methods can be used to compensate for a mutation in the human beta-globin gene in cells that have one or more mutations in the beta-globin gene or an expression control sequence thereof, for example mutations that result in the expression of the HbS form of hemoglobin. Compensating for the mutation includes, but is not limited to, increasing the amount of HbF and reducing the amount of HbS in the subject compared to untreated subjects or prior to treatment of a subject. In another embodiment, the method of treatment results in an increase in the ratio of HbF to HbS expressed in cells in a subject in need thereof. The methods can be used for treating sickle cell disease, for example sickle cell anemia, and other hemoglobinopathies or thalassemias as well as complications related to SCD, for example retinopathy.

In another embodiment, the present invention provides a method of inhibiting polymerization of HbS, of increasing dissolved oxygen levels in a subject's blood, of reducing levels of reactive oxygen species (ROS), or any combination thereof by administering a compound of the invention to a subject in need thereof.

In another embodiment, the present invention provides a method of reducing sickling in response to reduced air pressure, reduced barometric pressure, reduced partial pressure of oxygen or hypoxia, reducing incidences or rate of painful crises, reducing incidences or rate of painful crises requiring hospitalization, reducing the incidences of chest syndrome, reducing the number of transfusion events, reducing the number of units of blood transfused per event or any combination thereof by administering a compound of the invention to a subject in need thereof. The reduction of incidences or rate may be over a week, month, or year.

In another embodiment, the invention provides a method of treatment comprising administering a compound (or salt) of any one of embodiments 1 to 250 to a subject. In another embodiment, the invention provides a method of treatment comprising administering between 0.1 milligrams and 2 grams of a compound (or salt) of any one of embodiments 1 to 250 to a subject.

In each of the methods described above or below, a compound (or salt) of any of embodiments 1 to 250 may be administered to a subject as part of a pharmaceutically formulation, as described herein.

In each of the methods described herein, the method may further include the step of determining whether the subject has one or more genetic alterations associated with SCD or first determining whether the subject has biochemical or morphological alterations associated with SCD.

In each of the methods described herein, the method may further include the step of determining whether administration of a compound of the invention has increased expression of HbF, decreased biomarkers associated with SCD such ROS, or reduced the symptoms associated with SCD. The method may further comprise the step of administering a higher dose of a compound of the invention if the subject has not increased expression of HbF, does not have decreased biomarkers associated with SCD such ROS, or does not have reduced the symptoms associated with SCD.

### B. Treatments in Combination with HU or an Nrf2 Activator

Methods for treating SCD or complications thereof described herein may also include administering a compound of the invention in combination with or alternation with HU or an Nrf2 activator. The combination may be administered in amounts effective to induce or increase expression of HbF.

### C. Diseases to be Treated

The compounds of the invention and the combinations described herein can be used to treat subjects with one or more mutations in the beta-globin gene (HBB gene). Mutations in the beta globin gene can cause sickle cell disease, beta thalassemia, or related diseases or conditions thereof. As discussed in more detail below, mutations in the beta-globin gene can be identified before or after manifestations of a disease's clinical symptoms. The compositions can be administered to a subject with one or more mutations in the beta-globin gene before or after the onset of clinical symptoms. Therefore, in some embodiments, the compositions are administered to a subject that has been diagnosed with one or more mutations in the beta-globin gene, but does not yet exhibit clinical symptoms. In some embodiments, the compositions are administered to a subject that is exhibiting one or more symptoms of a disease, condition, or syndrome associated with, or caused by one or more mutations in the beta-globin gene.

### 1. Sickle Cell Disease

Sickle cell disease (SCD) typically arises from a mutation substituting thymine for adenine in the sixth codon of the beta-chain gene of hemoglobin (i.e., GAG to GTG of the HBB gene). This mutation causes glutamate to valine substitution in position 6 of the Hb beta chain. The resulting Hb, referred to as HbS, has the physical properties of forming polymers under deoxy conditions. SCD is typically an autosomal recessive disorder. Therefore, in some embodiments, the disclosed compositions and methods are used to treat a subject homozygous for an autosomal recessive mutation in beta-chain gene of hemoglobin (i.e., homozygous for sickle cell hemoglobin (HbS)). Also referred to as HbSS disease or sickle cell anemia (the most common form), subjects homozygote for the S globin typically exhibit a severe or moderately severe phenotype and have the shortest survival of the hemoglobinopathies.

Sickle cell trait or the carrier state is the heterozygous form characterized by the presence of around 40% HbS, absence of anemia, inability to concentrate urine (isosthenuria), and hematuria. Under conditions leading to hypoxia, it may become a pathologic risk factor. Accordingly, in some embodiments, the disclosed compositions and methods are used to treat a subject heterozygous for an autosomal recessive mutation in the beta-chain gene of hemoglobin (i.e., heterozygous for HbS).

### 2. Beta-Thalassemia

Beta-thalassemias (ß-thalassemias) are a group of inherited blood disorders caused by a variety of mutational mechanisms that result in a reduction or absence of synthesis of ß-globin and leading to accumulation of aggregates of unpaired, insoluble α-chains that cause ineffective erythropoiesis, accelerated red cell destruction, and severe anemia. Subjects with beta-thalassemia exhibit variable phenotypes ranging from severe anemia to clinically asymptomatic individuals. The genetic mutations present in ß-thalassemias are diverse, and can be caused by a number of different mutations. The mutations can involve a single base substitution or deletions or inserts within, near or upstream of the ß-globin gene. For example, mutations occur in the promoter regions preceding the beta-globin genes or cause production of abnormal splice variants. Examples of thalassemias include thalassemia minor, thalassemia intermedia, and thalassemia major.

### 3. Sickle Cell Related Disorders

Although carriers of sickle cell trait do not suffer from SCD, individuals with one copy of HbS and one copy of a gene that codes for another abnormal variant of hemoglobin, such as HbC or Hb beta-thalassemia, have a less severe form of the disease. A subject that is a double heterozygote for HbS and HbC (HbSC disease) is typically characterized by symptoms of moderate clinical severity. Another common structural variant of beta-globin is hemoglobin E or hemoglobin E (HbE). A subject that is a double heterozygote for HbS and HbE has HbS/HbE syndrome, which usually causes a phenotype similar to HbS/b+ thalassemia, discussed below.

Some mutations in the beta-globin gene can cause other structural variations of hemoglobin or can cause a deficiency in the amount of ß-globin being produced. These types of mutations are referred to as beta-thalassemia mutations. The absence of beta-globin is referred to as beta-zero (ß-0) thalassemia. A subject that is a double heterozygote for HbS and ß-0 thalassemia (i.e., HbS/ß-0 thalassemia) can suffer symptoms clinically indistinguishable from sickle cell anemia. A reduced amount of beta-globin is referred to as ß-plus (ß+) thalassemia. A subject that is a double heterozygote for HbS and ß+ thalassemia (i.e., HbS/ß+ thalassemia) can have mild-to-moderate severity of clinical symptoms with variability among different ethnicities. Rare combinations of HbS with other abnormal hemoglobins include HbD Los Angeles, G-Philadelphia, HbO Arab, and others.

Therefore, in some embodiments, the disclosed compositions and methods are used to treat a subject with an HbS/ß-0 genotype, an HbS/ß+genotype, an HBSC genotype, an HbS/HbE genotype, an HbD Los Angeles genotype, a G-Philadelphia genotype, or an abHbO Arab genotype.

As discussed above, retinopathy due to SCD can also be treated by administering an effective amount of a compound of the invention, optionally in combination or alternation with HU or with an Nrf2 activator in amounts effective to induce expression of HbF in retinal cells, for example in RPE cells. Administration of a compound of the invention optionally in combination with HU or with an Nrf2 activator may reduce or inhibit the formation of occlusions in the peripheral retina of a sickle cell patient.

### 4. Non-Erythroid Cell Related Disorders

Although red blood cells are the primary producers of hemoglobin, reports indicate that other, non-hematopoietic cells, including, but not limited to, macrophage, retinal pigment cells, and alveolar epithelial cells such as alveolar type II (ATII) cells and Clara cells also synthesize hemoglobin. In some embodiments, the compositions disclosed herein are used to increase HbF expression in non-erythroid cells including, but not limited to, macrophage, retinal pigment cells, and alveolar epithelial cells such as alveolar type II (ATII) cells and Clara cells. In some embodiments, the compositions disclosed herein are used to increase HbF expression in non-erythroid cells at interfaces where oxygen-carbon dioxide diffusion occurs, including, but not limited to the eyes and lungs. In some embodiments, the compositions are used to induce, increase, or enhance hemoglobin synthesis in retinal pigment cells in an effective amount to prevent, reduce, or alleviate one or more symptoms of age-related macular degeneration or diabetic retinopathy.

### D. Symptoms of SCD, Beta-Thalassemias, and Related Disorders

In some embodiments, the compositions disclosed herein are administered to a subject in an amount effective to treat one or more symptoms of sickle cell disease, a beta-thalassemia, or a related disorder.

Beta-thalassemia can include symptoms such as anemia, fatigue and weakness, pale skin or jaundice, protruding abdomen with enlarged spleen and liver, dark urine, abnormal facial bones, poor growth, and poor appetite.

In subjects with sickle cell disease, or a related disorder, physiological changes in RBCs can result in a disease with the following signs: (1) hemolytic anemia; (2) vaso-occlusive crisis; and (3) multiple organ damage from microinfarcts, including heart, skeleton, spleen, and central nervous system.

### III. Compositions for Use in Treating SCD and Related Disorders

### A. Compounds of the Invention (Compounds of Formula (I)))

A compound of Formula (I) has the structure shown below

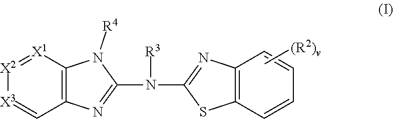

wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloalkyl, heterocyclyl, —C₁₋₆ alkylene-C₃₋₁₀ heterocyclyl, phenyl, heteroaryl, or NR^{h}R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

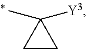

where Y³ is cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from Rx, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloalkyl, where the alkyl, alkylene, and cycloalkyl groups are optionally substituted one or more times with substituents independently selected from Rz;
R⁴ is —C₁₋₆ alkyl or —C₁₋₆ alkylene-C₃₋₁₀ cycloalkyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloalkyl, where the alkyl, alkylene, and cycloalkyl groups are optionally substituted one or more times with substituents independently selected from R^{x};
R^{a} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{d},
   i) —S(O)_{w}—R^{d},
   j) —S(O)₂O—R^{d},
   k) —NR^{d}R^{e},
   l) —C(O)—R^{d},
   m) —C(O)—O—R^{d},
   n) —OC(O)—R^{d},
   o) —C(O)NR^{d}R^{e},
   p) —C(O)-heterocyclyl,
   q) —NR^{d}C(O)R^{e},
   r) —OC(O)NR^{d}R^{e},
   s) —NR^{d}C(O)OR^{d}, or
   t) —NR^{d}C(O)NR^{d}R^{e},
   where the alkyl, cycloalkyl, and heterocyclyl groups are optionally substituted one or more times with substituents independently selected from R^{y};
R^{b} is
   a) -halogen,
   b) —C₁₋₆ alkyl,
   c) —C₃₋₁₀ cycloalkyl,
   d) -heterocyclyl,
   e) -phenyl,
   f) -heteroaryl,
   g) -cyano,
   h) —CF₃,
   i) —OCF₃,
   j) —O—R^{f},
   k) —S(O)_{w}—R^{f},
   l) —S(O)₂O—R^{f},
   m) —NR^{f}R^{g},
   n) —C(O)—R^{f},
   o) —C(O)—O—R^{f},
   p) —OC(O)—R^{f},
   q) —C(O)NR^{f}R^{g},
   r) —C(O)-heterocyclyl,
   s) —NR^{f}C(O)R^{g},
   t) —OC(O)NR^{f}R^{g},
   u) —NR^{f}C(O)OR^{f}, or
   v)—NR^{f}C(O)NR^{f}R^{g},
   where the alkyl, cycloalkyl, heterocyclyl, phenyl, and heteroaryl groups are optionally substituted one or more times with substituents independently selected from R^{z};
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O) NR^{h}R^{k},
   p) —C(O)-heterocyclyl,
   q) —NR^{h}C(O)R^{k},
   r) —OC(O)NR^{h}R^{k},
   s) —NR^{h}C(O)OR^{k},
   t) —NR^{h}C(O)NR^{h}R^{k},
   u) —NR^{h}S(O)_{w}R^{k},
   v) -phenyl,
   w) -heteroaryl, or
   x) —O—(C₁₋₄ alkylene)—O—(C₁₋₄ alkylene)-N(R^{h})C(O)—OR^{k},
   where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃-10 cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R²;
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
   a) -halogen,
   b) —NH₂,
   c) -cyano,
   d) -carboxy,
   e) -hydroxy,
   f) -thiol,
   g) —CF₃,
   h) —OCF₃,
   i) —C(O)—NH₂,
   j) —S(O)₂—NH₂,
   k) oxo,
   l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   z) —S(O)₂—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂, or
   bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂;
R^{x} is
   a) —R^{x}
   b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   d) —O-phenyl,
   e) —O-heteroaryl,
   f) —C(O)-phenyl,
   g) —C(O)-heteroaryl,
   h) —C(O)—O-phenyl, or
   i) —C(O)—O-heteroaryl;
R^{z} is
   a) —R^{y}
   b) -phenyl,
   c) -heteroaryl;
   d) —O-phenyl,
   e) —O-heteroaryl,
   f) —C(O)-phenyl,
   g) —C(O)-heteroaryl,
   h) —C(O)—O-phenyl, or
   i) —C(O)—O-heteroaryl;
v is an integer from 0 to 4, and
w is an integer from 0 to 2.
Embodiment 2: A compound according to embodiment 1 wherein
G is hydrogen, —C₁₋₈ alkyl, —C₃₋₁₀ cycloalkyl, —C₁₋₆ alkylene-C₃₋₁₀ cycloalkyl, heterocyclyl, phenyl, heteroaryl, or NR^{h}R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

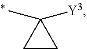

where Y³ is -cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
R^{c} is
   a) -halogen,
   b) —C₁₋₆ alkyl,
   c) —C₃₋₁₀ cycloalkyl,
   d) -heterocyclyl,
   e)-cyano,
   f)—CF₃,
   g)—OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   q) —NR^{h}C(O)R^{k},
   r) —OC(O)NR^{h}R^{k},
   s) —NR^{h}C(O)OR^{k},
   t) —NR^{h}C(O)NR^{h}R^{k},
   u) -phenyl,
   v) -heteroaryl, or
   w) —O—(C₁₋₄ alkylene)—O—(C₁₋₄ alkylene)-N(R^{h})C(O)—OR^{k},
   where the alkylene, alkyl, cycloalkyl, heterocyclyl, phenyl, and heteroaryl groups are optionally substituted one or more times with substituents independently selected from R^{x};
R^{h} and R^{k} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{y} is
   a) -halogen,
   b) —NH₂,
   c) -cyano,
   d) -carboxy,
   e) —C₁₋₆ alkyl, optionally substituted one or more times with halogen,
   f) -heterocyclyl, optionally substituted one or more times with halogen,
   g) —C₃₋₁₀ cycloalkyl, optionally substituted one or more times with halogen,
   h) —O—C₁₋₆ alkyl, optionally substituted one or more times with halogen,
   i) —O—C₃₋₁₀ cycloalkyl, optionally substituted one or more times with halogen,
   j) -hydroxy,
   k) -thiol,
   l) —CF₃,
   m) —OCF₃,
   n) —C(O)—C₁₋₆ alkyl, optionally substituted one or more times with halogen,
   o) —C(O)—O—C₁₋₆ alkyl, optionally substituted one or more times with halogen,
   p) —S—C₁₋₆ alkyl, optionally substituted one or more times with halogen, or
   q) —S(O)₂—C₁₋₆ alkyl, optionally substituted one or more times with halogen.
Embodiment 3: A compound according to embodiment 2, wherein R³ is hydrogen.
Embodiment 4: A compound according to embodiment 2, wherein R³ is methyl.
Embodiment 5: A compound according to any one of embodiments 2 to 4, wherein X¹ is ═N—.
Embodiment 6: A compound according to any one of embodiments 2 to 4, wherein X¹ is ═CH—.
Embodiment 7: A compound according to any one of embodiments 2 to 6, wherein v is an integer from 0 to 2.
Embodiment 8: A compound according to any one of embodiments 2 to 6, wherein v is 0 or 1.
Embodiment 9: A compound according to any one of embodiments 2 to 6, wherein vis 1.
Embodiment 10: A compound according to any one of embodiments 2 to 6, wherein v is 1, and R² is attached at either the 5-position or the 6-position of the benzothiazole ring.
Embodiment 11: A compound according to any one of embodiments 2 to 6, wherein v is 1, and R² is attached at the 6-position of the benzothiazole ring.
Embodiment 12: A compound according to any one of embodiments 2 to 68, wherein v is 2, and one R² is attached at the 6-position of the benzothiazole ring.
Embodiment 13: A compound according to any one of embodiments 2 to 6, wherein v is 2, and R² is attached at the 5-position and the 6-position of the benzothiazole ring.
Embodiment 14: A compound according to any one of embodiments 2 to 13, wherein R² is -halogen, —C₁₋₆ alkyl, —CF₃, —OCF₃, —O—R^{f}, or —S(O).—R^{f}, where the alkyl group is optionally substituted one or more times with substituents independently selected from R^{z}.
Embodiment 15: A compound according to any one of embodiments 2 to 13, wherein R² is -halogen, -methyl, —CF₃, —OCF₃, —SCF₃, —O-heteroaryl, or —S(O)₂—CH₃.
Embodiment 16: A compound according to any one of embodiments 2 to 13, wherein R² is selected from —Cl, —F, —CF₃, and —OCF₃.
Embodiment 17: A compound according to any one of embodiments 2 to 13, wherein R² is —OCF₃.
Embodiment 18: A compound according to any one of embodiments 2 to 13, wherein R² is —CF₃.
Embodiment 19: A compound according to any one of embodiments 2 to 13, wherein R² is —F.
Embodiment 20: A compound according to any one of embodiments 2 to 13, wherein R² is —Cl.
Embodiment 21: A compound according to any one of embodiments 2 to 20, wherein R⁴ is -methyl, -ethyl, -n-propyl, -isopropyl, -n-butyl, -sec-butyl, -isobutyl, -tert-butyl, —(CH₂)₁₋₂—OCH₃, —(CH₂)₁₋₂—F, —(CH₂)₁₋₂—Cl, —(CH₂)₁₋₂—OCF₃, —(CH₂)₁₋₂—NH₂, —(CH₂)₁₋₂—CN, —(CH₂)₁₋₂—OH, —(CH₂)₁₋₂—CF₃, —(CH₂)₁₋₂—CO₂H, —(CH₂)₁₋₂—SH, —(CH₂)₁₋₂—SCH₃, —(CH₂)₁₋₂—S(O)₂CH₃, —(CH₂)₁₋₂—OCH₂CH₃, —(CH₂)₁₋₂—SCH₂CH₃, —(CH₂)₁₋₂—S(O)₂CH₂CH₃, —(CH₂)₁₋₂—NH—CH₃, or —(CH₂)₁₋₂—N(CH₃)₂.
Embodiment 22: A compound according to any one of embodiments 2 to 21, wherein R⁴ is -methyl, -ethyl, -isopropyl, -isobutyl, —CH₂CH₂—OCH₃, —CH₂CH₂—F, or —CH₂CH₂—NH₂.
Embodiment 23: A compound according to any one of embodiments 2 to 22, wherein R⁴ is -methyl, -ethyl, -isopropyl, or -isobutyl.
Embodiment 24: A compound according to any one of embodiments 2 to 23, wherein R⁴ is -methyl.
Embodiment 25: A compound according to any one of embodiments 2 to 23, wherein R⁴ is -ethyl.
Embodiment 26: A compound according to any one of embodiments 2 to 21, wherein R⁴ is —(CH₂)₂—OCH₃, —(CH₂)₂—F, —(CH₂)₂—Cl, —(CH₂)₂—OCF₃, —(CH₂)₂—NH₂, —(CH₂)₂—CN, —(CH₂)₂—OH, —(CH₂)₂—CF₃, —(CH₂)₂—CO₂H, —(CH₂)₂—SH, —(CH₂)₂—SCH₃, or (CH₂)₂—S(O)₂CH₃.
Embodiment 27: A compound according to any one of embodiments 2 to 26, wherein R¹ is selected from hydrogen, —OCH₃, —F, —Cl, —NH₂, -cyano, —OH, —CF₃, —OCF₃, —SH, —S—C₁₋₆ alkyl, —S(O)₂—C₁₋₆ alkyl, —CO₂H, —NH—C₁₋₆ alkyl, —N(C₁₋₆ alkyl)₂, and —NH—C₁₋₆ alkyl.
Embodiment 28: A compound according to any one of embodiments 2 to 26, wherein R¹ is selected from-OCH₃, —F, —CF₃, —OCF₃, —N(CH₃)₂, —N(CH₂CH₃)₂, and —N(CH₃)(CH₂CH₃).
Embodiment 29: A compound according to any one of embodiments 2 to 26, wherein R¹ is selected from hydrogen, —OCH₃, and —F.
Embodiment 30: A compound according to any one of embodiments 2 to 26, wherein R¹ is hydrogen.
Embodiment 31: A compound according to any one of embodiments 2 to 30, wherein
G is hydrogen, —C₁₋₈ alkyl, —C₃₋₁₀ cycloalkyl, —C₁₋₆ alkylene-C₃₋₈ cycloalkyl, heterocyclyl, or NR^{h}R^{k}, where the alkyl, alkylene, cycloalkyl, and heterocyclyl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

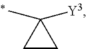

where CH₂CH(Y³)CH₃, —CH(Y³)CH₃, —CH₂C(Y³)(CH₃)₂, —C(Y³)(CH₃)₂, or Y³ is -cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, or heterocyclylene optionally substituted one or more times with substituents independently selected from R^{x}; or the group -L-G is -cyano;
R¹ is hydrogen or R^{a};
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   q) —NR^{h}C(O)R^{k},
   r) —OC(O)NR^{h}R^{k},
   s) —NR^{h}C(O)OR^{k},
   t) —NR^{h}C(O)NR^{h}R^{k}, or
   u) —O—(C₁₋₄ alkylene)—O—(C₁₋₄ alkylene)-N(R^{h})C(O)—OR^{k},
   where the alkylene, alkyl, cycloalkyl, and heterocyclyl groups are optionally substituted one or more times with substituents independently selected from R^{x};
R^{h} and R^{k} are independently hydrogen, C₁₋₆ alkyl, or C₃₋₁₀ cycloalkyl, where the alkyl, and cycloalkyl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{x} is R^{y}.
Embodiment 32: A compound according to any one of embodiments 2 to 31, wherein -L-G is not-cyano.
Embodiment 33: A compound according to any one of embodiments 2 to 32, wherein -L-G is —C(O)NR^{h}R^{k}.
Embodiment 34: A compound according to any one of embodiments 2 to 32, wherein L is —C(O)N(R⁶)— or —C(O)—O—.
Embodiment 35: A compound according to any one of embodiments 2 to 32, wherein L is —C(O)N(R⁶)—.
Embodiment 36: A compound according to any one of embodiments 2 to 32, wherein L is not —CH₂—C(O)N(R⁶)—.
Embodiment 37: A compound according to any one of embodiments 2 to 32, wherein L is —C(O)—O—.
Embodiment 38: A compound according to any one of embodiments 2 to 32, wherein L is —C(O)—.
Embodiment 39: A compound according to any one of embodiments 2 to 32, wherein L is —S(O)₂—.
Embodiment 40: A compound according to any one of embodiments 2 to 30, wherein L is heteroarylene optionally substituted one or more times with substituents independently selected from R^{x}.
Embodiment 41: A compound according to any one of embodiments 2 to 40, wherein R⁶ is hydrogen.
Embodiment 42: A compound according to any one of embodiments 2 to 40, wherein R⁶ is hydrogen or -methyl.
Embodiment 43: A compound according to any one of embodiments 2 to 42, wherein G is hydrogen, —C₁₋₈ alkyl, —C₃₋₁₀ cycloalkyl, or —C₁₋₆ alkylene-C₃₋₈ cycloalkyl, where the alkyl, cycloalkyl, and alkylene groups are optionally substituted one or more times with substituents independently selected from R^{x}.
Embodiment 44: A compound according to any one of embodiments 2 to 42, wherein G is —H, -methyl, -ethyl, -n-propyl, -isopropyl, -isobutyl, —CH₂Y³, —CH₂CH₂Y³, —CH₂CH₂CH₂Y³, —CH(CH₃)CH₂Y³, —CH CH(Y³)CH₃, —CH(Y³)CH₃, —CH₂C(Y³)(CH₃)₂, or —C(Y³)(CH₃)₂, where Y³ is -cyclopropyl, —CF₃, —OCF₃, —OCH₃, —OCH₂CH₃, —F, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, —NH—C(O)—CH₃, —NH—C(O)—CH₂CH₃, or C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, or —N(CH₂CH₃)₂.
Embodiment 45: A compound according to any one of embodiments 2 to 42, wherein G is -methyl, -ethyl, -n-propyl, -isopropyl, or -isobutyl, where each is optionally substituted one or more times with substituents independently selected from-CF₃, —OCF₃, —OCH₃, —OCH₂CH₃, —F, —OH, —O(CH₂)₂—OH, —O(CH₂)₂—F, —SCH₃, —SCH₂CH₃, —NH—CH₃, —NH—CH₂CH₃, and —N(CH₃)₂.
Embodiment 46: A compound according to any one of embodiments 2 to 42, wherein G is H.
Embodiment 47: A compound according to any one of embodiments 2 to 42, wherein G is C₁₋₈ alkyl optionally substituted one or more times with halogen.
Embodiment 48: A compound according to any one of embodiments 2 to 42, wherein G is C₃₋₁₀ cycloalkyl optionally substituted one or more times with halogen.
Embodiment 49: A compound according to any one of embodiments 2 to 42, wherein G is heterocyclyl optionally substituted one or more times with halogen.
Embodiment 50: A compound according to any one of embodiments 2 to 42, wherein G is —C₁₋₆ alkylene-C₃₋₁₀ cycloalkyl optionally substituted one or more times with halogen.
Embodiment 51: A compound according to any one of embodiments 2 to 42, wherein G is NR^{h}R^{k}.
Embodiment 52: A compound according to any one of embodiments 2 to 42, wherein G is —CH₂—R^{c}.
Embodiment 53: A compound according to any one of embodiments 2 to 42, wherein G is —CH₂CH₂—R^{c}.
Embodiment 54: A compound according to any one of embodiments 2 to 42, wherein G is —CH₂CH₂CH₂—R^{c}.
Embodiment 55: A compound according to any one of embodiments 2 to 42, wherein G is —CH(CH₃)CH₂R^{c}.
Embodiment 56: A compound according to any one of embodiments 2 to 42, wherein G is —CH₂CH (R^{c}) CH₃.
Embodiment 57: A compound according to any one of embodiments 2 to 42, wherein G is —CH (R^{c}) CH₃.
Embodiment 58: A compound according to any one of embodiments 2 to 42, wherein G is —CH₂C(R^{c})(CH₃)₂.
Embodiment 59: A compound according to any one of embodiments 2 to 42, wherein G is —C(R^{e})(CH₃)₂.
Embodiment 60: A compound according to any one of embodiments 2 to 42, wherein G is imidazol-2-yl, thiazol-2yl, oxazol-2-yl, pyrazol1-yl, furan-2yl, thiophen-2-yl, pyrrol-1-yl, 1H-1,2,4-triazolyl-3-yl, 5-methyl-1H-1,2,4-triazolyl-3-yl, —(CH₂)₁₋₃-(imidazol-2-yl), —(CH₂)₁₋₃-(thiazol-2yl), —(CH₂)₁₋₃-(oxazol-2-yl), —(CH₂)₁₋₃-(pyrazol1-yl), —(CH₂)₁₋₃-(furan-2yl), —(CH₂)₁₋₃-(thiophen-2-yl), —(CH₂)₁₋₃-(pyrrol-1-yl), —(CH₂)₁₋₃-(1H-1,2,4-triazolyl-3-yl), or —(CH₂)₁₋₃-(5-methyl-1H-1,2,4-triazolyl-3-yl).
Embodiment 61: A compound according to any one of embodiments 2 to 60, wherein the compound is in its free (non-salted) form.
Embodiment 62: A compound according to any one of embodiments 2 to 60, wherein the compound is in the form of a pharmaceutically acceptable salt.
Embodiment 63: A compound according to any one of embodiments 1 to 62, wherein any “heterocyclyl” group present in the compound is selected from the group consisting of: azetidin-1-yl, azetidin-2-yl, azetidin-3-yl, pyrrolidin-1-yl, pyrrolidin-2-yl, pyrrolidin-3-yl, tetrahydrofuran-2-yl, tetrahydrofuran-3-yl, tetrahydrothiophen-2-yl, tetrahydrothiophen-3-yl, pyrazolidin-1-yl, pyrazolidin-3-yl, pyrazolidin-4-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolodin-5-yl, isothiazolidin-2-yl, isothiazolidin-3-yl, isothiazolidin-4-yl, isothiazolodin-5-yl, 1,3-dioxolan-2-yl, 1,3-dioxolan-4-yl, 1,3-oxathiolan-2-yl, 1,3-oxathiolane-4-yl, 1,3-oxathiolan-5-yl, 1,2-dithiolan-3-yl, 1,2-dithiolan-4-yl, 1,3-dithiolan-2-yl, 1,3-dithiolan-4-yl, piperidin-1-yl, piperidin-2-yl, piperidin-3-yl, piperidin-4-yl, tetrahydropyran-2-yl, tetrahydropyran-3-yl, tetrahydropyran-4-yl, thian-2-yl, thian-3-yl, thian-4-yl, piperazin-1-yl, piperazin-2-yl, morpholin-2-yl, morpholin-3-yl, morpholin-4-yl, thiomorpholin-2-yl, thiomorpholin-3-yl, thiomorpholin-4-yl, 1,4-dioxan-2-yl, 1,3-dioxan-2-yl, 1,3-dioxan-4-yl, 1,3-dioxan-5-yl, 1,4-dithian-2-yl, 1,3-dithian-2-yl, 1,3-dithian-4-yl, 1,3-dithian-5-yl, 1,2-dithian-3-yl, 1,2-dithian-4-yl, azepan-1-yl, azepan-2-yl, azepan-3-yl, and azepan-4-yl, where each of these named rings may optionally be substituted one or more times with substituents independently selected from halogen, —NH₂, cyano, carboxy, C₁₋₄ alkyl, C₃₋₁₀ cycloalkyl, hydroxyl, thiol, —CF₃, —OCF₃, —O—C₁₁ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, and —C(O)N(C₁₋₄ alkyl)₂, and where any nitrogen atom in any of these named rings may optionally be oxidized when chemically feasible, and where any sulfur atom in any of these named rings may optionally be oxidized once or twice when chemically feasible.
Embodiment 64: A compound according to any one of embodiments 1 to 63, wherein any “heteroaryl” group present in the compound is selected from the group consisting of: 1H-pyrrol-1-yl, 1H-pyrrol-2-yl, 1H-pyrrol-3-yl, furan-2-yl, furan-3-yl, thiophen-2-yl, thiophen-3-yl, 1H-imidazol-1-yl, 1H-imidazol-2-yl, 1H-imidazol-4-yl, 1H-imidazol-5-yl, 1H-pyrazol-1-yl, 1H-pyrazol-3-yl, 1H-pyrazol-4-yl, 1H-pyrazol-5-yl, oxazol-2-yl, oxazol-4-yl, oxazol-5-yl, thiazol-2-yl, thiazol-4-yl, thiazol-5-yl, isoxazol-3-yl, isoxazol-4-yl, isoxazol-5-yl, isothiazol-3-yl, isothiazol-4-yl, isothiazol-5-yl, 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl, 1H-1,2,4-triazol-5-yl, furazan-3-yl, pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1H-indol-1-yl, 1H-indol-2-yl, 1H-indol-3-yl, 2H-isoindol-1-yl, 2H-isoindol-2-yl, quinolin-2-yl, quinolin-3-yl, quinolin-4-yl, isoquinolin-1-yl, isoquinolin-3-yl, isoquinolin-4-yl, benzoxazol-2-yl, benzothiazol-2-yl, 1H-benzimidazol-1-yl, 1H-benzimidazol-2-yl, benzofuran-2-yl, benzofuran-3-yl, benzothiophen-2-yl, and benzothiophen-3-yl, where each of these named rings may optionally be substituted one or more times with substituents independently selected from halogen, —NH₂, cyano, carboxy, C₁₋₄ alkyl, C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₁ alkyl, —C(O)O—C₁₋₄ alkyl, —C(O)NH₂, —C(O)NH—C₁₋₄ alkyl, —C(O)N(C₁₋₄ alkyl)₂, and phenyl.
Embodiment 65: A compound according to any one of embodiments 1 to 64, wherein any “heteroarylene” group present in the compound is selected from the group consisting of: 1H-pyrrol-2,5-diyl, furan-2,5-diyl, thiophen-2,5-diyl, 1H-imidazol-2,4-diyl, 1H-imidazol-2,5-diyl, oxazol-2,4-diyl, oxazol-2,5-diyl, thiazol-2,4-diyl, thiazol-2,5-diyl, 1H-1,2,4-triazol-3,5-diyl, and 2H-isoindol-1,3-diyl, where each of these named rings may optionally be substituted one or more times with substituents independently selected from halogen, —NH₂, cyano, carboxy, —C₁₋₄ alkyl, —C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₄ alkyl, —C(O)N(C₁₋₄ alkyl)₂, and phenyl.
Embodiment 66: A compound according to embodiment 1.
Embodiment 67: A compound according to embodiment 66, wherein R³ is hydrogen.
Embodiment 68: A compound according to embodiment 66, wherein R³ is methyl.
Embodiment 69: A compound according to embodiment 66, wherein R³ is ethyl.
Embodiment 70: A compound according to embodiment 66, wherein R³ is isopropyl.
Embodiment 71: A compound according to any one of embodiment 66 to 70, wherein X¹ is ═N—.
Embodiment 72: A compound according to any one of embodiments 66 to 70, wherein X¹ is ═CH—.
Embodiment 73: A compound according to any one of embodiments 66 to 72, wherein v is 0, 1 or 2.
Embodiment 74: A compound according to any one of embodiments 66 to 72, wherein vis 1 or 2.
Embodiment 75: A compound according to any one of embodiments 66 to 72, wherein vis 1.
Embodiment 76: A compound according to any one of embodiments 66 to 72, wherein v is 1, and R² is attached at either the 5-position or the 6-position of the benzothiazole ring.
Embodiment 77: A compound according to any one of embodiments 66 to 72, wherein v is 1, and R² is attached at the 6-position of the benzothiazole ring.
Embodiment 78: A compound according to any one of embodiments 66 to 72, wherein v is 2, and one R² is attached at the 6-position of the benzothiazole ring.
Embodiment 79: A compound according to any one of embodiments 66 to 72, wherein v is 2, and R² is attached at the 5-position and the 6-position of the benzothiazole ring.
Embodiment 80: A compound according to any one of embodiments 66 to 79, wherein R² is -halogen, —C₁₋₆ alkyl, —CF₃, —OCF₃, —O—R^{f}, or —S(O).—R^{f}, where the alkyl group is optionally substituted one or more times with substituents independently selected from R^{z}.
Embodiment 81: A compound according to any one of embodiments 66 to 79, wherein R² is -halogen, -methyl, ethyl, isopropyl, —OCH₃, —OCH₂CH₃, —OCH(CH₃)₂, —CF₃, —OCF₃, —SCF₃, —S(O)₂—CH₃, —O-phenyl, —O-(2-pyridyl), —O-(3-pyridyl), or —O-(4-pyridyl).
Embodiment 82: A compound according to any one of embodiments 66 to 79, wherein R² is -halogen, -methyl, ethyl, isopropyl, —OCH₃, —OCH₂CH₃, —OCH(CH₃)₂, —CF₃, —OCF₃, —SCF₃, —S(O)₂—CH₃, or —O-(3-pyridyl).
Embodiment 83: A compound according to any one of embodiments 66 to 79, wherein R² is —Cl, —F, —CF₃, or —OCF₃.
Embodiment 84: A compound according to any one of embodiments 66 to 79, wherein R² is —OCF₃.
Embodiment 85: A compound according to any one of embodiments 66 to 79, wherein R² is —CF₃.
Embodiment 86: A compound according to any one of embodiments 66 to 79, wherein R² is —F.
Embodiment 87: A compound according to any one of embodiments 66 to 79, wherein R² is —Cl.
Embodiment 88: A compound according to any one of embodiments 66 to 79, wherein R² is —SO₂CH₃.
Embodiment 89: A compound according to any one of embodiments 66 to 79, wherein R² is methyl, ethyl, or isopropyl.
Embodiment 90: A compound according to any one of embodiments 66 to 79, wherein R² is methyl.
Embodiment 91: A compound according to any one of embodiments 66 to 79, wherein R² is —OCH₂CH₃.
Embodiment 92: A compound according to any one of embodiments 66 to 79, wherein R² is —O-phenyl.
Embodiment 93: A compound according to any one of embodiments 66 to 79, wherein R² is —O-(2-pyridyl), —O-(3-pyridyl), or —O-(4-pyridyl).
Embodiment 94: A compound according to any one of embodiments 66 to 79, wherein R² is —O-(3-pyridyl).
Embodiment 95: A compound according to any one of embodiments 66 to 94, wherein R⁴ is -methyl, -ethyl, -n-propyl, -isopropyl, -n-butyl, -sec-butyl, -isobutyl, -tert-butyl, —(CH₂)₁₋₂—OCH₃, —(CH₂)₁₋₂—F, —(CH₂)₁₋₂—Cl, —(CH₂)₁₋₂—OCF₃, —(CH₂)₁₋₂—NH₂, —(CH₂)₁₋₂—CN, —(CH₂)₁₋₂—OH, —(CH₂)₁₋₂—CF₃, —(CH₂)₁₋₂—CO₂H, —(CH₂)₁₋₂—SH, —(CH₂)₁₋₂—SCH₃, —(CH₂)₁₋₂—S(O)₂CH₃, —(CH₂)₁₋₂—OCH₂CH₃, —(CH₂)₁₋₂—SCH₂CH₃, —(CH₂)₁₋₂—S(O)₂CH₂CH₃, —(CH₂)₁₋₂—NH—CH₃, or —(CH₂)₁₋₂—N(CH₃)₂.
Embodiment 96: A compound according to any one of embodiments 66 to 94, wherein R⁴ is -methyl, -ethyl, -isopropyl, -isobutyl, —CH₂CH₂—OCH₃, —CH₂CH₂—F, —CH₂CH₂—NH₂, or —CH₂CH₂—NH—CH₃.
Embodiment 97: A compound according to any one of embodiments 66 to 94, wherein R⁴ is -methyl, -ethyl, -isopropyl, or -isobutyl.
Embodiment 98: A compound according to any one of embodiments 66 to 94, wherein R⁴ is methyl.
Embodiment 99: A compound according to any one of embodiments 66 to 94, wherein R⁴ is -ethyl.
Embodiment 100: A compound according to any one of embodiments 66 to 94, wherein R⁴ is -isopropyl.
Embodiment 101: A compound according to any one of embodiments 66 to 94, wherein R⁴ is -isobutyl.
Embodiment 102: A compound according to any one of embodiments 66 to 94, wherein R⁴ is —CH₂CH₂—OCH₃.
Embodiment 103: A compound according to any one of embodiments 66 to 94, wherein R⁴ is —CH₂CH₂—F.
Embodiment 104: A compound according to any one of embodiments 66 to 94, wherein R⁴ is —CH₂CH₂—NH₂.
Embodiment 105: A compound according to any one of embodiments 66 to 94, wherein R⁴ is —CH₂CH₂—NH—CH₃.
Embodiment 106: A compound according to any one of embodiments 66 to 105, wherein R¹ is hydrogen, —OCH₃, —F, —Cl, —NH₂, -cyano, —OH, —CF₃, —OCF₃, —SH, —S—C₁₋₆ alkyl, —S(O)₂—C₁₋₆ alkyl, —CO₂H, —NH—C₁₋₆ alkyl, —N(C₁₋₆ alkyl)₂, or —NH—C₁₋₆ alkyl.
Embodiment 107: A compound according to any one of embodiments 66 to 105, wherein R¹ is —OCH₃, —F, —CF₃, —OCF₃, —N(CH₃)₂, —N(CH₂CH₃)₂, or —N(CH₃)(CH₂CH₃).
Embodiment 108: A compound according to any one of embodiments 66 to 105, wherein R¹ is hydrogen, —OCH₃, or —F.
Embodiment 109: A compound according to any one of embodiments 66 to 105, wherein R¹ is hydrogen.
Embodiment 110: A compound according to any one of embodiments 66 to 105, wherein R¹ is —F.
Embodiment 111: A compound according to any one of embodiments 66 to 105, wherein R¹ is —OCH₃.
Embodiment 112: A compound according to any one of embodiments 66 to 105 wherein R¹ is —N(CH₂CH₃)₂.
Embodiment 113: A compound according to any one of embodiments 66 to 112, wherein
G is hydrogen, —C₁₋₈ alkyl, —C₃₋₁₀ cycloalkyl, —C₁₋₆ alkylene-C₃₋₁₀ cycloalkyl, heterocyclyl, —C₁₋₆ alkylene-C₃₋₁₀ heterocyclyl, or NR^{h}R^{k}, where the alkyl, alkylene, cycloalkyl, and heterocyclyl groups are optionally substituted one or more times with substituents independently selected from R^{e}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

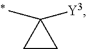

where Y³ is cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, or heterocyclylene optionally substituted one or more times with substituents independently selected from R^{x}; or the group -L-G is -cyano;
R¹ is hydrogen or R^{a};
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂₀—R^{h},
   k) —NR^{h}R^{k},
   l) —C(O)—R^{h},
   m) —C(O)—O—R^{h},
   n) —OC(O)—R^{h},
   o) —C(O)NR^{h}R^{k},
   p) —C(O)-heterocyclyl,
   q) —NR^{h}C(O)R^{k},
   r) —OC(O)NR^{h}R^{k},
   s) —NR^{h}C(O)OR^{k},
   t) —NR^{h}C(O)NR^{h}R^{k},
   u) —NR^{h}S(O)_{w}R^{k}, or
   v) —O—(C₁₋₄ alkylene)—O—(C₁₋₄ alkylene)-N(R^{h})C(O)—OR^{k},
   where the alkylene, alkyl, cycloalkyl, and heterocyclyl groups are optionally substituted one or more times with substituents independently selected from R^{x};
R^{h} and R^{k} independently are hydrogen, C₁₋₆ alkyl, C₃-10 cycloalkyl, or heterocyclyl, where the alkyl, cycloalkyl, and heterocyclyl groups are optionally substituted one or more times with substituents independently selected from R^{x}; or, if R^{h} and R^{k} are both attached to the same nitrogen atom, together with that nitrogen atom may optionally form a heterocyclic ring selected from azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{x} is R^{y}.
Embodiment 114: A compound according to any one of embodiments 66 to 112, wherein -L-G is not-cyano.
Embodiment 115: A compound according to any one of embodiments 66 to 112, wherein L is —C(O)N(R⁶)—.
Embodiment 116: A compound according to embodiment 115 wherein R⁶ is hydrogen.
Embodiment 117: A compound according to embodiment 115 wherein R⁶ is methyl.
Embodiment 118: A compound according to embodiment 117 wherein G is —N(CH₃)₂.
Embodiment 119: A compound according to any one of embodiments 66 to 112, wherein -L-G is —C(O)NR^{h}R^{k}.
Embodiment 120: A compound according to embodiment 119, wherein NR^{h}R^{k} is pyrrolidino, piperidino, piperazino, 4-methyl-piperazino, or morpholino, where each of the foregoing is optionally substituted once with —(CH₂)₁₋₃—OH.
Embodiment 121: A compound according to embodiment 120, wherein NR^{h}R^{k} is pyrrolidino, 4-(2-hydroxyethyl)-piperazino, or 4-(3-hydroxypropyl)-piperidino.
Embodiment 122: A compound according to embodiment 119, wherein NR^{h}R^{k} is N [(CH₂)₂—OH]₂.
Embodiment 123: A compound according to any one of embodiments 66 to 114, wherein L is not —CH₂—C(O)N(R⁶)—.
Embodiment 124: A compound according to any one of embodiments 66 to 123, wherein L is not heterocyclylene.
Embodiment 125: A compound according to any one of embodiments 66 to 112, wherein L is —S(O)₂—.
Embodiment 126: A compound according to embodiment 125, wherein G is methyl or —CF₃.
Embodiment 127: A compound according to any one of embodiments 66 to 112, wherein L is heteroarylene optionally substituted one or more times with substituents independently selected from R^{x}.
Embodiment 128: A compound according to embodiment 127, wherein -L-G is imidazol-2-yl, 1,2,4-triazol-3-yl, or 5-methyl-1,2,4-triazol-3-yl.
Embodiment 129: A compound according to any one of embodiments 66 to 112, wherein L is —C(O)—O—.
Embodiment 130: A compound according to embodiment 129, wherein G is hydrogen, or —C_{1.8} alkyl, where the alkyl group is optionally substituted one or more times with substituents independently selected from R^{c}.
Embodiment 131: A compound according to embodiment 130, wherein G is methyl or ethyl.
Embodiment 132: A compound according to embodiment 130, wherein G is hydrogen.
Embodiment 133: A compound according to any one of embodiments 66 to 116, wherein G is —C₁₋₈ alkyl, —C₃₋₁₀ cycloalkyl, —C₁₋₆ alkylene-C₃₋₁₀ cycloalkyl, heterocyclyl, or —C₁₋₆ alkylene-C₃₋₁₀ heterocyclyl, where the alkyl, alkylene, cycloalkyl, and heterocyclyl groups are optionally substituted one or more times with substituents independently selected from R^{c}.
Embodiment 134: A compound according to embodiment 133, wherein G is —C₁₋₈ alkyl optionally substituted one or more times with substituents independently selected from R^{c}.
Embodiment 135: A compound according to embodiment 134, wherein G is methyl, ethyl, isopropyl, n-propyl, n-butyl, sec-butyl, or isobutyl.
Embodiment 136: A compound according to embodiment 134, wherein G is methyl, ethyl, or n-propyl.
Embodiment 137: A compound according to embodiment 134, wherein G is 2-fluoroethyl, 2,2-difluoroethyl, or 2,2,2-trifluoroethyl.
Embodiment 138: A compound according to embodiment 134, wherein G is 2-cyanoethyl.
Embodiment 139: A compound according to embodiment 134, wherein G is —C₁₋₈ alkyl substituted once by-C(O)—O—R^{h}.
Embodiment 140: A compound according to embodiment 139, wherein G is —CH₂—C(O)—O—R^{h}
Embodiment 141: A compound according to embodiment 140, wherein R^{h} is hydrogen or methyl.
Embodiment 142: A compound according to embodiment 139, wherein G is —CH₂CH₂—C(O)—O—R^{h}.
Embodiment 143: A compound according to embodiment 142, wherein R^{h} is hydrogen or methyl.
Embodiment 144: A compound according to embodiment 139, wherein G is —C(CH₃)₂—C(O)—O—R^{h}
Embodiment 145: A compound according to embodiment 144, wherein R^{h} is hydrogen or methyl.
Embodiment 146: A compound according to embodiment 139, wherein G is —CH(CH₃)—C(O)—O—R^{h}.
Embodiment 147: A compound according to embodiment 146, wherein R^{h} is hydrogen or methyl.
Embodiment 148: A compound according to embodiment 134, wherein G is —C₁₋₈ alkyl substituted once by —C(O)NR^{h}R^{k}
Embodiment 149: A compound according to embodiment 148, wherein G is CH₂—C(O)—NR^{h}R^{k}.
Embodiment 150: A compound according to embodiment 149, wherein NR^{h}R^{k} is methylamino, dimethylamino, or diethylamino.
Embodiment 151: A compound according to embodiment 149, wherein NR^{h}R^{k} is thiomorpholino or 1,1-dioxothiomorpholino.
Embodiment 152: A compound according to embodiment 149, wherein NR^{h}R^{k} is morpholino, pyrrolidino, piperidino, piperazino, or 4-methylpiperazino.
Embodiment 153: A compound according to embodiment 149, wherein NR^{h}R^{k} is pyrrolidino, 3-hydroxy-pyrrolidino, 3-methoxy-pyrrolidino, 3-amino-pyrrolidino, 3-(methylamino)-pyrrolidino, 3-(dimethylamino)-pyrrolidino, 2-(hydroxymethyl)-pyrrolidino, 2-(dimethylaminocarbonyl)-pyrrolidino or 3,4-dihydroxy-pyrrolidino.
Embodiment 154: A compound according to embodiment 149, wherein NR^{h}R^{k} is piperazino, 4-methylpiperazino, 4-(methylsulfonyl)-piperazino, or 4-(dimethylaminosulfonyl)-piperazino.
Embodiment 155: A compound according to embodiment 149, wherein NR^{h}R^{k} is piperidino, 3-hydroxypiperidino, 4-hydroxypiperidino, 2-(hydroxymethyl)-piperidino, 3-(hydroxymethyl)-piperidino, 4-(hydroxymethyl)-piperidino, 3-methoxy-piperidino, 4-(methoxymethyl)-piperidino, 4-(fluoromethyl)-piperidino, 4-(trifluoromethyl)-piperidino, 4-cyano-piperidino, 4-carbamoyl-piperidino, 4-(methylamino)-piperidino, 4-(dimethylamino)-piperidino, 4-(methylaminomethyl)-piperidino, or 4-(dimethylaminomethyl)-piperidino.
Embodiment 156: A compound according to embodiment 149, wherein NR^{h}R^{k} is NHR^{k}, where R^{k} is 2-hydroxypropyl, 2-(methylsulfonyl)-ethyl, tetrahydrofuran-3-yl, tetrahydropyran-4-yl, 1-methylpiperidin-4-yl, piperidin-3-yl, or 1-methylpiperidin-3-yl.
Embodiment 157: A compound according to embodiment 149, wherein NR^{h}R^{k} is N(CH₃) R^{k}, where R^{k} is 2-hydroxyethyl, tetrahydropyran-4-yl, pyrrolidin-3-yl, 1-methylpyrrolidin-3-yl, or piperazin-3-yl.
Embodiment 158: A compound according to embodiment 149, wherein NR^{h}R^{k} is N(CH₂CH₂OH)₂.
Embodiment 159: A compound according to embodiment 148, wherein G is —(CH₂)₂₋₃—C(O)—N(CH₃)₂.
Embodiment 160: A compound according to embodiment 148, wherein G is —(CH₂)₃—C(O)-(4-methylpiperazino).
Embodiment 161: A compound according to embodiment 148, wherein G is —CH(CH₃)—C(O)—NR^{h}R^{k}, where NR^{h}R^{k} is methylamino, dimethylamino, 4-methylpiperazino, or morpholino.
Embodiment 162: A compound according to embodiment 148, wherein G is —C(CH₃)₂—C(O)—N(CH₃)₂.
Embodiment 163: A compound according to embodiment 134, wherein G is —CH—[C(O)—N(CH₃)₂]—[CH₂OH], —CH—[C(O)—N(CH₃)₂]—[(CH₂)₄—NH₂], or —CH—[C(O)—N(CH₃)₂]—[(CH₂)₃—N(CH₃)₂].
Embodiment 164: A compound according to embodiment 134, wherein G is —C₁₋₈ alkyl substituted once by —O—R^{h}.
Embodiment 165: A compound according to embodiment 164, wherein G is —(CH₂)₂—O—R^{h}.
Embodiment 166: A compound according to embodiment 165, wherein R^{h} is hydrogen, methyl, or ethyl.
Embodiment 167: A compound according to embodiment 165, wherein R^{h} is trifluoromethyl, 2-fluoroethyl, 3-fluoropropyl, or 2,2-difluoroethyl.
Embodiment 168: A compound according to embodiment 165, wherein R^{h} is tetrahydrofuran-2-ylmethyl.
Embodiment 169: A compound according to embodiment 165, wherein R^{h} is 2-hydroxyethyl.
Embodiment 170: A compound according to embodiment 165, wherein R^{h} is 3-hydroxypropyl.
Embodiment 171: A compound according to embodiment 165, wherein R^{h} is 2-methoxyethyl.
Embodiment 172: A compound according to embodiment 165, wherein R^{h} is 2-(2-hydroxyethoxy)-ethyl.
Embodiment 173: A compound according to embodiment 165, wherein R^{h} is 2-hydroxypropyl or 1-hydroxyprop-2-yl.
Embodiment 174: A compound according to embodiment 165, wherein R^{h} is 2-cyanoethyl, 2-(methylcarbonylamino)-ethyl, or 2-(methylsulfonylamino)-ethyl.
Embodiment 175: A compound according to embodiment 165, wherein R^{h} is 2-aminoethyl, 2-(methylamino)-ethyl, or 2-(dimethylamino)-ethyl.
Embodiment 176: A compound according to embodiment 165, wherein R^{h} is carbamoylmethyl.
Embodiment 177: A compound according to embodiment 164, wherein G is -(CH₂) 3-O—R^{h}.
Embodiment 178: A compound according to embodiment 177, wherein R^{h} is hydrogen, methyl, or ethyl.
Embodiment 179: A compound according to embodiment 177, wherein R^{h} is 2-hydroxyethyl.
Embodiment 180: A compound according to embodiment 164, wherein G is —(CH₂)₄—OH, —(CH₂)₅—OH, —CH₂C(CH₃)₂—OH, —CH₂C(CH₃)₂—OCH₃, —CH₂C(CH₃)₂—CH₂—OH, —CH(CH₃)—CH₂—OCH₃, —(CH₂)₃C(CH₃)₂—CH₂—OH, —(CH₂)₂CH(CH₃)—CH₂—OH, or —(CH₂)₂CH(CH₃)—OH.
Embodiment 181: A compound according to embodiment 164, wherein G is —CH₂CH(CH₃)—O—R^{h}.
Embodiment 182: A compound according to embodiment 181, wherein R^{h} is hydrogen, methyl, or ethyl.
Embodiment 183: A compound according to embodiment 134, wherein G is —CH₂—CH (OH)—CH₂—OH.
Embodiment 184: A compound according to embodiment 134, wherein G is —C₁₋₈ alkyl substituted once by-NR^{h}R^{k}.
Embodiment 185: A compound according to embodiment 184, wherein G is —(CH₂)₂—NR^{h}R^{k}
Embodiment 186: A compound according to embodiment 185, wherein NR^{h}R^{k} is amino, methylamino, or dimethylamino.
Embodiment 187: A compound according to embodiment 185, wherein NR^{h}R^{k} is methylcarbonylamino.
Embodiment 188: A compound according to embodiment 185, wherein NR^{h}R^{k} is (dimethylamino) methylcarbonylamino, hydroxymethylcarbonylamino, or 1-hydroxyethylcarbonylamino.
Embodiment 189: A compound according to embodiment 185, wherein NR^{h}R^{k} is methylsulfonylamino.
Embodiment 190: A compound according to embodiment 185, wherein NR^{h}R^{k} is piperidino, 4-hydroxypiperidino, or 3-hydroxypiperidino.
Embodiment 191: A compound according to embodiment 185, wherein NR^{h}R^{k} is piperidino, 4,4-difluoropiperidino, or 3,3-difluoropiperidino.
Embodiment 192: A compound according to embodiment 185, wherein NR^{h}R^{k} is 2-oxo-pyrrolidino, 2-oxo-imidazolidino, or 3-oxo-piperazino.
Embodiment 193: A compound according to embodiment 185, wherein NR^{h}R^{k} is piperazino, 4-methylpiperazino, morpholino, or 1,1-dioxothiomorpholino.
Embodiment 194: A compound according to embodiment 184, wherein G is —(CH₂)₃—NR^{h}R^{k}.
Embodiment 195: A compound according to embodiment 194, wherein NR^{h}R^{k} is amino, dimethylamino, or diethylamino.
Embodiment 196: A compound according to embodiment 194, wherein NR^{h}R^{k} is piperidino, 4-methylpiperazino, or morpholino.
Embodiment 197: A compound according to embodiment 184, wherein G is —(CH₂)+—NR^{h}R^{k}.
Embodiment 198: A compound according to embodiment 197, wherein NR^{h}R^{k} is amino, dimethylamino, or diethylamino.
Embodiment 199: A compound according to embodiment 133, wherein G is —C₁₋₆ alkylene-heterocyclyl, where the alkylene and heterocyclyl groups are optionally substituted one or more times with substituents independently selected from R^{c}.
Embodiment 200: A compound according to embodiment 199, wherein G is —CH₂-heterocyclyl, where the heterocyclyl group is optionally substituted once with a substituent selected from R^{c}.
Embodiment 201: A compound according to embodiment 200, wherein the heterocyclyl group is tetrahydropyran-4-yl, tetrahydrofuran-2-yl, 1,4-dioxan-2-yl, morpholin-2-yl, tetrahydropyran-2-yl, piperidin-4-yl, 1-(2-hydroxyethyl)-piperidin-4-yl, 1-(dimethylaminomethylcarbonyl)-piperidin-4-yl, piperazin-2-yl, or 1-methyl-piperazin-2-yl.
Embodiment 202: A compound according to embodiment 133, wherein G is C₃₋₁₀ cycloalkyl optionally substituted one or more times with substituents independently selected from R^{c}.
Embodiment 203: A compound according to embodiment 202, wherein G is 4-hydroxy-cyclohexyl, 4-carboxy-cyclohexyl, or 4-(dimethylaminocarbonyl)-cyclohexyl.
Embodiment 204: A compound according to embodiment 202, wherein G is 1-carboxy-cyclopropyl, 1-(ethoxycarbonyl)-cyclopropyl, or 1-(dimethylamino-carbonyl)-cyclopropyl.
Embodiment 205: A compound according to embodiment 133, wherein G is C₁₋₆ alkylene-C₃₋₁₀ cycloalkyl, where the alkylene and cycloalkyl groups are optionally substituted one or more times with substituents independently selected from R^{c}.
Embodiment 206: A compound according to embodiment 205, wherein G is —CH₂-(4-hydroxy-cyclohexyl).
Embodiment 207: A compound according to embodiment 205, wherein G is —(CH₂)₂-(4-hydroxy-cyclohexyl).
Embodiment 208: A compound according to embodiment 205, wherein G is —CH₂-[4-(hydroxymethyl)-cyclohexyl].
Embodiment 209: A compound according to embodiment 133, wherein G is heterocyclyl optionally substituted one or more times with substituents independently selected from R^{c}.
Embodiment 210: A compound according to embodiment 209, wherein G is piperidin-4-yl, 1-methyl-piperidin-4-yl, 1-carboxy-piperidin-4-yl, 1-(methylsulfonyl)-piperidin-4-yl, 1-(2-hydroxyethyl)-piperidin-4-yl, 1-(dimethyl-aminocarbonyl) piperidin-4-yl, or 1-(dimethylaminomethylcarbonyl)-piperidin-4-yl.
Embodiment 211: A compound according to embodiment 209, wherein G is piperidin-3-yl or 1-(dimethylaminomethylcarbonyl)-piperidin-3-yl.
Embodiment 212: A compound according to embodiment 209, wherein G is 1,1-dioxo-tetrahydrothiophen-3-yl.
Embodiment 213: A compound according to embodiment 209, wherein G is pyrrolidin-3-yl, 1-methyl-pyrrolidin-3-yl, 1-(2-hydroxyethyl)-pyrrolidin-3-yl, 1-(2-hydroxypropyl)-pyrrolidin-3-yl, 1-(2-hydroxy-2-methylpropyl)-pyrrolidin-3-yl, 1-(1-hydroxyethylcarbonyl)-pyrrolidin-3-yl, 1-(2-carboxyethyl)-pyrrolidin-3-yl, or 1-(2-methylsulfonylamino-ethyl)-pyrrolidin-3-yl.
Embodiment 214: A compound according to embodiment 134, wherein G is —C₁₋₈ alkyl substituted once by —S—R^{h}.
Embodiment 215: A compound according to embodiment 214, wherein G is —(CH₂)₂—S—R^{h}.
Embodiment 216: A compound according to embodiment 215, wherein R^{h} is methyl or ethyl.
Embodiment 217: A compound according to embodiment 215, wherein R^{h} is 2-hydroxyethyl.
Embodiment 218: A compound according to embodiment 214, wherein G is —(CH₂)₃—S—R^{h}.
Embodiment 219: A compound according to embodiment 218, wherein R^{h} is methyl.
Embodiment 220: A compound according to embodiment 134, wherein G is —C₁₋₈ alkyl substituted once by —SO₂—R^{h}.
Embodiment 221: A compound according to embodiment 220, wherein G is —(CH₂)₂—SO₂—R^{h}.
Embodiment 222: A compound according to embodiment 221, wherein R^{h} is methyl or ethyl.
Embodiment 223: A compound according to embodiment 221, wherein R^{h} is 2-hydroxyethyl.
Embodiment 224: A compound according to embodiment 220, wherein G is —(CH₂) 3—SO₂—R^{h}.
Embodiment 225: A compound according to embodiment 224, wherein R^{h} is methyl.
Embodiment 226: A compound according to embodiment 133 wherein G is —CH(CH₃)—NR^{h}R^{k}, where NR^{h}R^{k} is pyrrolidino, piperidino, 4-methylpiperazino, morpholino, or dimethylamino.
Embodiment 227: A compound according to embodiment 133 wherein G is 1-(2-hydroxypropyl)-pyrrolodin-3-yl or 1-(1-hydroxyethylcarbonyl)-pyrrolidin-3-yl.
Embodiment 228: A compound according to embodiment 133 wherein G is 1-(dimethylaminomethylcarbonyl)-piperidin-4-yl.
Embodiment 229: A compound according to embodiment 133 wherein G is —(CH₂)₃₋₅—OH.
Embodiment 230: A compound according to embodiment 133 wherein G is 4-hydroxy-cyclohexylmethyl.
Embodiment 231: A compound according to embodiment 133 wherein G is —(CH₂)₂—NHC(O)—CH₂—N(CH₃)₂.
Embodiment 232: A compound according to embodiment 133 wherein G is 4-hydroxy-cyclohexylmethyl.
Embodiment 233: A compound according to embodiment 133 wherein G is —CH₂—C(O)—NR^{h}R^{k}, where NR^{h}R^{k} is 3-hydroxy-pyrrolidino or 3-(dimethyl-amino)-pyrrolidino.
Embodiment 234: A compound according to embodiment 133 wherein G is —CH₂—C(O)—NR^{h}R^{k}, where NR^{h}R^{k} is morpholino.
Embodiment 235: A compound according to embodiment 133 wherein G is —CH₂—C(O)—NR^{h}R^{k}, where NR^{h}R^{k} is 4-hydroxy-piperidino, 4-methoxy-piperidino, 4-(hydroxymethyl)-piperidino, 3-hydroxy-piperidino, 3-methoxy-piperidino, 3-(hydroxymethyl)-piperidino, or 4,4-difluoropiperidino.
Embodiment 236: A compound according to embodiment 133 wherein G is —CH₂—C(O)—NR^{h}R^{k}, where NR^{h}R^{k} is dimethylamino.
Embodiment 237: A compound according to embodiment 133 wherein G is —(CH₂)₂—O—(CH₂)₂—OH.
Embodiment 238: A compound according to embodiment 133 wherein G is —(CH₂)₂—O—(CH₂)₂—OCH₃.
Embodiment 239: A compound according to embodiment 133 wherein G is —CH₂—CH(CH₃)—OH.
Embodiment 240: A compound according to any one of embodiments 66 to 112, wherein L is C(O)NH, and G is C₁₋₈ alkyl substituted once by a heteroaryl group, where the heteroaryl group is optionally substituted one or more times with substituents independently selected from R^{x}.
Embodiment 241: A compound according to embodiment 240, wherein G is —CH₂-(2-furyl), —CH₂-(2-thienyl), —CH₂-(2-oxazolyl), or —CH₂-(2-thiazolyl).
Embodiment 242: A compound according to embodiment 240, wherein G is —(CH₂)₂₋₃-(1-pyrrolyl), —(CH₂)₂₋₃-(1-pyrazolyl), or —(CH₂)₂₋₃-(1-imidazolyl).
Embodiment 243: A compound according to any one of embodiments 66 to 112, wherein L is C(O)NH, and G is C₁₋₈ alkyl substituted once by a phenyl group, where the phenyl group is optionally substituted one or more times with substituents independently selected from R^{x}.
Embodiment 244: A compound according to embodiment 243, wherein G is —(—CH₂)₁₋₂-(4-hydroxyphenyl) or —(—CH₂)₁₋₂-(4-methoxy-3-hydroxyphenyl).
Embodiment 245: A compound according to any one of embodiments 66 to 112, wherein L is C(O)NH, and G is —CH₂—C(O)NH—CH₂-(4-hydroxyphenyl).
Embodiment 246: A compound according to any one of embodiments 66 to 112, wherein L is C(O)NH, and G is —CH₂—C(O)-[4-(pyrimidin-2-yloxy)-piperidino].
Embodiment 247: A compound according to any one of embodiments 1 to 246, wherein X² is ═C(R¹)— and X³ is ═C(-L-G)-.
Embodiment 248: A compound according to any one of embodiments 1 to 247, wherein the compound is in the form of a free acid or a free base.
Embodiment 249: A compound according to any one of embodiments 1 to 247, wherein the compound is in the form of a pharmaceutically acceptable salt.
Embodiment 250: A compound according to embodiment 1, wherein the compound is a compound from Table A or a pharmaceutically acceptable salt thereof.

TABLE A

| No. | Name |
|---|---|
| 1 | 1-Methyl-2-(6-trifluoromethoxy-benzothiazol-2-ylamino)-1H- |
| | benzoimidazole-5-carboxylic acid methyl amide |
| 2 | 1-Methyl-2-(6-trifluoromethoxy-benzothiazol-2-ylamino)-1H- |
| | benzoimidazole-5-carboxylic acid methyl ester |
| 3 | 1-Methyl-2-(6-trifluoromethoxy-benzothiazol-2-ylamino)-1H- |
| | benzoimidazole-5-carboxylic acid |
| 4 | 1-Methyl-2-(6-trifluoromethoxy-benzothiazol-2-ylamino)-1H- |
| | benzoimidazole-5-carboxylic acid (2-ethoxy-ethyl)-amide |
| 5 | 1-Methyl-2-(6-trifluoromethoxy-benzothiazol-2-ylamino)-1H- |
| | benzoimidazole-5-carboxylic acid cyclopropylmethyl-amide |
| 6 | 1-Methyl-2-(6-trifluoromethoxy-benzothiazol-2-ylamino)-1H- |
| | benzoimidazole-5-carboxylic acid ethylamide |
| 7 | [1-Methyl-2-(6-trifluoromethoxy-benzothiazol-2-ylamino)-1H- |
| | benzoimidazol-5-yl]-pyrrolidin-1-yl-methanone |
| 8 | 1-Methyl-2-(6-trifluoromethoxy-benzothiazol-2-ylamino)-1H- |
| | benzoimidazole-5-carboxylic acid (2-methoxy-ethyl)-amide |
| 9 | 1-Methyl-2-(6-trifluoromethoxy-benzothiazol-2-ylamino)-1H- |
| | benzoimidazole-5-carboxylic acid (2-fluoro-ethyl)-amide |
| 10 | 1-Methyl-2-(6-trifluoromethoxy-benzothiazol-2-ylamino)-1H- |
| | benzoimidazole-5-carboxylic acid (2-hydroxy-ethyl)-amide |
| 11 | 1-Methyl-2-(6-trifluoromethoxy-benzothiazol-2-ylamino)-1H- |
| | benzoimidazole-5-carboxylic acid (3-pyrazol-1-yl-propyl)-amide |
| 12 | 1-Methyl-2-(6-trifluoromethoxy-benzothiazol-2-ylamino)-1H- |
| | benzoimidazole-5-carboxylic acid propylamide |
| 13 | 1-Methyl-2-(6-trifluoromethoxy-benzothiazol-2-ylamino)-1H- |
| | benzoimidazole-5-carboxylic acid (3-hydroxy-propyl)-amide |
| 14 | 1-Methyl-2-(6-trifluoromethoxy-benzothiazol-2-ylamino)-1H- |
| | benzoimidazole-5-carboxylic acid (3-ethoxy-propyl)-amide |
| 15 | 1-Methyl-2-(6-trifluoromethoxy-benzothiazol-2-ylamino)-1H- |
| | benzoimidazole-5-carboxylic acid morpholin-4-ylamide |
| 16 | 1-Methyl-2-(6-trifluoromethoxy-benzothiazol-2-ylamino)-1H- |
| | benzoimidazole-5-carboxylic acid (2,2,2-trifluoro-ethyl)-amide |
| 17 | 1-Methyl-2-(6-trifluoromethoxy-benzothiazol-2-ylamino)-1H- |
| | benzoimidazole-5-carboxylic acid (tetrahydro-pyran-4-ylmethyl)-amide |
| 18 | 1-Methyl-2-(6-trifluoromethoxy-benzothiazol-2-ylamino)-1H- |
| | benzoimidazole-5-carboxylic acid (tetrahydro-furan-2-ylmethyl)-amide |
| 19 | 1-Methyl-2-(6-trifluoromethoxy-benzothiazol-2-ylamino)-1H- |
| | benzoimidazole-5-carboxylic acid (3-methoxy-propyl)-amide |
| 20 | 1-Methyl-2-(6-trifluoromethoxy-benzothiazol-2-ylamino)-1H- |
| | benzoimidazole-5-carboxylic acid (2-methoxy-1-methyl-ethyl)-amide |
| 21 | 1-Methyl-2-(6-trifluoromethoxy-benzothiazol-2-ylamino)-1H- |
| | benzoimidazole-5-carboxylic acid (2-hydroxy-propyl)-amide |
| 22 | 1-Methyl-2-(6-trifluoromethoxy-benzothiazol-2-ylamino)-1H- |
| | benzoimidazole-5-carboxylic acid (2-methoxy-2-methyl-propyl)-amide |
| 23 | 1-Methyl-2-(6-trifluoromethyl-benzothiazol-2-ylamino)-1H- |
| | benzoimidazole-5-carboxylic acid methyl ester |
| 24 | 1-Methyl-2-(6-trifluoromethyl-benzothiazol-2-ylamino)-1H- |
| | benzoimidazole-5-carboxylic acid |
| 25 | 1-Methyl-2-(6-trifluoromethyl-benzothiazol-2-ylamino)-1H- |
| | benzoimidazole-5-carboxylic acid (2-methoxy-ethyl)-amide |
| 26 | 1-Methyl-2-(6-trifluoromethyl-benzothiazol-2-ylamino)-1H- |
| | benzoimidazole-5-carboxylic acid ethylamide |
| 27 | 1-Methyl-2-(6-trifluoromethyl-benzothiazol-2-ylamino)-1H- |
| | benzoimidazole-5-carboxylic acid (2-hydroxy-ethyl)-amide |
| 28 | 2-(6-Chloro-benzothiazol-2-ylamino)-1-methyl-1H-benzoimidazole-5- |
| | carboxylic acid methyl ester |
| 29 | 2-(6-Chloro-benzothiazol-2-ylamino)-1-methyl-1H-benzoimidazole-5- |
| | carboxylic acid |
| 30 | 2-(6-Chloro-benzothiazol-2-ylamino)-1-methyl-1H-benzoimidazole-5- |
| | carboxylic acid (2-hydroxy-ethyl)-amide |
| 31 | 2-(6-Chloro-benzothiazol-2-ylamino)-1-methyl-1H-benzoimidazole-5- |
| | carboxylic acid (2-methoxy-ethyl)-amide |
| 32 | 2-(6-Chloro-benzothiazol-2-ylamino)-1-methyl-1H-benzoimidazole-5- |
| | carboxylic acid ethylamide |
| 33 | 2-(5,6-Difluoro-benzothiazol-2-ylamino)-1-methyl-1H-benzoimidazole-5- |
| | carboxylic acid methyl ester |
| 34 | 2-(5,6-Difluoro-benzothiazol-2-ylamino)-1-methyl-1H-benzoimidazole-5- |
| | carboxylic acid |
| 35 | 2-(5,6-Difluoro-benzothiazol-2-ylamino)-1-methyl-1H-benzoimidazole-5- |
| | carboxylic acid ethylamide |
| 36 | 2-(5,6-Difluoro-benzothiazol-2-ylamino)-1-methyl-1H-benzoimidazole-5- |
| | carboxylic acid (2-hydroxy-ethyl)-amide |
| 37 | 2-(5,6-Difluoro-benzothiazol-2-ylamino)-1-methyl-1H-benzoimidazole-5- |
| | carboxylic acid (2-methoxy-ethyl)-amide |
| 38 | 3-Methyl-2-(6-trifluoromethoxy-benzothiazol-2-ylamino)-3H- |
| | benzoimidazole-5-carboxylic acid methylamide |
| 39 | 6-Fluoro-1-methyl-2-(6-trifluoromethoxy-benzothiazol-2-ylamino)-1H- |
| | benzoimidazole-5-carboxylic acid (2-methoxy-ethyl)-amide |
| 40 | 6-Fluoro-1-methyl-2-(6-trifluoromethoxy-benzothiazol-2-ylamino)-1H- |
| | benzoimidazole-5-carboxylic acid methyl ester |
| 41 | 6-Fluoro-1-methyl-2-(6-trifluoromethoxy-benzothiazol-2-ylamino)-1H- |
| | benzoimidazole-5-carboxylic acid |
| 42 | 6-Fluoro-1-methyl-2-(6-trifluoromethoxy-benzothiazol-2-ylamino)-1H- |
| | benzoimidazole-5-carboxylic acid ethylamide |
| 43 | 1-Methyl-2-(6-trifluoromethoxy-benzothiazol-2-ylamino)-1H- |
| | benzoimidazole-5-carboxylic acid (2-morpholin-4-yl-ethyl)-amide |
| 44 | 1-Methyl-2-(6-trifluoromethoxy-benzothiazol-2-ylamino)-1H- |
| | benzoimidazole-5-carboxylic acid (2-trifluoromethoxy-ethyl)-amide |
| 45 | 1-Methyl-2-(6-trifluoromethoxy-benzothiazol-2-ylamino)-1H- |
| | benzoimidazole-5-carboxylic acid (2-hydroxy-2-methyl-propyl)-amide |
| 46 | 1-Methyl-2-(6-trifluoromethoxy-benzothiazol-2-ylamino)-1H- |
| | benzoimidazole-5-carboxylic acid [2-(2-hydroxy-ethoxy)-ethyl]-amide |
| 47 | 1-Methyl-2-(6-trifluoromethoxy-benzothiazol-2-ylamino)-1H- |
| | benzoimidazole-5-carboxylic acid [2-(2-fluoro-ethoxy)-ethyl]-amide |
| 48 | 1-Methyl-2-(6-trifluoromethoxy-benzothiazol-2-ylamino)-1H- |
| | benzoimidazole-5-carboxylic acid (furan-2-ylmethyl)-amide |
| 49 | 1-Methyl-2-(6-trifluoromethoxy-benzothiazol-2-ylamino)-1H- |
| | benzoimidazole-5-carboxylic acid ([1,4]dioxan-2-ylmethyl)-amide |
| 50 | 1-Methyl-2-(6-trifluoromethoxy-benzothiazol-2-ylamino)-1H- |
| | benzoimidazole-5-carboxylic acid ((S)-2-hydroxy-propyl)-amide |
| 51 | 1-Methyl-2-(6-trifluoromethoxy-benzothiazol-2-ylamino)-1H- |
| | benzoimidazole-5-carboxylic acid ((R)-2-hydroxy-propyl)-amide |
| 52 | 1-Methyl-2-(6-trifluoromethoxy-benzothiazol-2-ylamino)-1H- |
| | benzoimidazole-5-carboxylic acid (trans-4-hydroxy-cyclohexyl)-amide |
| 53 | 1-Methyl-2-(6-trifluoromethoxy-benzothiazol-2-ylamino)-1H- |
| | benzoimidazole-5-carboxylic acid [2-(tetrahydro-furan-2-ylmethoxy)- |
| | ethyl]-amide |
| 54 | 1-Methyl-2-(6-trifluoromethoxy-benzothiazol-2-ylamino)-1H- |
| | benzoimidazole-5-carboxylic acid (2-ethoxy-propyl)-amide |
| 55 | 2-({[1-Methyl-2-(6-trifluoromethoxy-benzothiazol-2-ylamino)-1H- |
| | benzoimidazole-5-carbonyl]-amino}-methyl)-morpholine-4-carboxylic acid |
| | tert-butyl ester |
| 56 | 1-Methyl-2-(6-trifluoromethoxy-benzothiazol-2-ylamino)-1H- |
| | benzoimidazole-5-carboxylic acid (morpholin-2-ylmethyl)-amide |
| | hydrochloride |
| 57 | 6-Fluoro-1-methyl-2-(6-trifluoromethoxy-benzothiazol-2-ylamino)-1H- |
| | benzoimidazole-5-carboxylic acid (2-ethoxy-ethyl)-amide |
| 58 | 6-Fluoro-1-methyl-2-(6-trifluoromethoxy-benzothiazol-2-ylamino)-1H- |
| | benzoimidazole-5-carboxylic acid dimethylcarbamoylmethyl-amide |
| 59 | 6-Fluoro-1-methyl-2-(6-trifluoromethoxy-benzothiazol-2-ylamino)-1H- |
| | benzoimidazole-5-carboxylic acid (2-morpholin-4-yl-ethyl)-amide |
| 60 | 6-Fluoro-1-methyl-2-(6-trifluoromethoxy-benzothiazol-2-ylamino)-1H- |
| | benzoimidazole-5-carboxylic acid (2-hydroxy-propyl)-amide |
| 61 | 6-Methoxy-1-methyl-2-(6-trifluoromethoxy-benzothiazol-2-ylamino)-1H- |
| | benzoimidazole-5-carboxylic acid methyl ester |
| 62 | 6-Methoxy-1-methyl-2-(6-trifluoromethoxy-benzothiazol-2-ylamino)-1H- |
| | benzoimidazole-5-carboxylic acid |
| 63 | 6-Methoxy-1-methyl-2-(6-trifluoromethoxy-benzothiazol-2-ylamino)-1H- |
| | benzoimidazole-5-carboxylic acid dimethylcarbamoyl-methyl-amide |
| 64 | 6-Methoxy-1-methyl-2-(6-trifluoromethoxy-benzothiazol-2-ylamino)-1H- |
| | benzoimidazole-5-carboxylic acid ethylamide |
| 65 | 6-Methoxy-1-methyl-2-(6-trifluoromethoxy-benzothiazol-2-ylamino)-1H- |
| | benzoimidazole-5-carboxylic acid (2-ethoxy-ethyl)-amide |
| 66 | 6-Methoxy-1-methyl-2-(6-trifluoromethoxy-benzothiazol-2-ylamino)-1H- |
| | benzoimidazole-5-carboxylic acid (2-morpholin-4-yl-ethyl)-amide |
| 67 | 6-Methoxy-1-methyl-2-(6-trifluoromethoxy-benzothiazol-2-ylamino)-1H- |
| | benzoimidazole-5-carboxylic acid (2-methoxy-ethyl)-amide |
| 68 | 6-Methoxy-1-methyl-2-(6-trifluoromethoxy-benzothiazol-2-ylamino)-1H- |
| | benzoimidazole-5-carboxylic acid (2-hydroxy-propyl)-amide |
| 69 | 6-Diethylamino-1-methyl-2-(6-trifluoromethoxy-benzothiazol-2-ylamino)- |
| | 1H-benzoimidazole-5-carboxylic acid methyl ester |
| 70 | 6-Diethylamino-1-methyl-2-(6-trifluoromethoxy-benzothiazol-2-ylamino)- |
| | 1H-benzoimidazole-5-carboxylic acid |
| 71 | 3-Methyl-2-(6-trifluoromethoxy-benzothiazol-2-ylamino)-3H-imidazo[4,5- |
| | b]pyridine-6-carboxylic acid ethyl ester |
| 72 | 3-Methyl-2-(6-trifluoromethoxy-benzothiazol-2-ylamino)-3H-imidazo[4,5- |
| | b]pyridine-6-carboxylic acid |
| 73 | 3-Methyl-2-(6-trifluoromethoxy-benzothiazol-2-ylamino)-3H-imidazo[4,5- |
| | b]pyridine-6-carboxylic acid (2-methoxy-ethyl)-amide |
| 74 | 3-Methyl-2-(6-trifluoromethoxy-benzothiazol-2-ylamino)-3H-imidazo[4,5- |
| | b]pyridine-6-carboxylic acid dimethylcarbamoylmethyl-amide |
| | 3-Methyl-2-(6-trifluoromethoxy-benzothiazol-2-ylamino)-3H-imidazo[4,5- |
| 75 | b]pyridine-6-carboxylic acid (2-ethoxy-ethyl)-amide |
| 76 | 3-Methyl-2-(6-trifluoromethoxy-benzothiazol-2-ylamino)-3H-imidazo[4,5- |
| | b]pyridine-6-carboxylic acid ethylamide |
| 77 | 3-Methyl-2-(6-trifluoromethoxy-benzothiazol-2-ylamino)-3H-imidazo[4,5- |
| | b]pyridine-6-carboxylic acid (2-morpholin-4-yl-ethyl)-amide |
| 78 | 3-Methyl-2-(6-trifluoromethoxy-benzothiazol-2-ylamino)-3H-imidazo[4,5- |
| | b]pyridine-6-carboxylic acid (2-hydroxy-propyl)-amide |
| 79 | {[1-Methyl-2-(6-trifluoromethoxy-benzothiazol-2-ylamino)-1H- |
| | benzoimidazole-5-carbonyl]-amino}-acetic acid methyl ester |
| 80 | 1-Methyl-2-(6-trifluoromethoxy-benzothiazol-2-ylamino)-1H- |
| | benzoimidazole-5-carboxylic acid dimethylcarbamoylmethyl-amide |
| 81 | 1-Methyl-2-(6-trifluoromethoxy-benzothiazol-2-ylamino)-1H- |
| | benzoimidazole-5-carboxylic acid ((S)-1-ethylcarbamoyl-ethyl)-amide |
| 82 | 1-Methyl-2-(6-trifluoromethoxy-benzothiazol-2-ylamino)-1H- |
| | benzoimidazole-5-carboxylic acid (2-dimethylamino-ethyl)-amide |
| 83 | {[1-Methyl-2-(6-trifluoromethoxy-benzothiazol-2-ylamino)-1H- |
| | benzoimidazole-5-carbonyl]-amino}-acetic acid |
| 84 | 1-Methyl-2-(6-trifluoromethyl-benzothiazol-2-ylamino)-1H- |
| | benzoimidazole-5-carboxylic acid methylamide |
| 85 | 1-Methyl-2-(6-trifluoromethyl-benzothiazol-2-ylamino)-1H- |
| | benzoimidazole-5-carboxylic acid (2-ethoxy-ethyl)-amide |
| 86 | 2-(5,6-Difluoro-benzothiazol-2-ylamino)-1-methyl-1H-benzoimidazole-5- |
| | carboxylic acid methylamide |
| 87 | 2-(5,6-Difluoro-benzothiazol-2-ylamino)-1-methyl-1H-benzoimidazole-5- |
| | carboxylic acid (2-ethoxy-ethyl)-amide |
| 88 | 2-(6-Chloro-benzothiazol-2-ylamino)-1-methyl-1H-benzoimidazole-5- |
| | carboxylic acid methylamide |
| 89 | 2-(6-Chloro-benzothiazol-2-ylamino)-1-methyl-1H-benzoimidazole-5- |
| | carboxylic acid (2-ethoxy-ethyl)-amide |
| 90 | 1-Methyl-2-(6-trifluoromethoxy-benzothiazol-2-ylamino)-1H- |
| | benzoimidazole-5-carboxylic acid (1-methanesulfonyl-piperidin-4-yl)-amide |
| 91 | {[1-Methyl-2-(6-trifluoromethoxy-benzothiazol-2-ylamino)-1H- |
| | benzoimidazole-5-carbonyl]-amino}-acetic acid tert-butyl ester |
| 92 | 4-{[1-Methyl-2-(6-trifluoromethoxy-benzothiazol-2-ylamino)-1H- |
| | benzoimidazole-5-carbonyl]-amino}-piperidine-1-carboxylic acid tert-butyl ester |
| 93 | 1-Methyl-2-(6-trifluoromethoxy-benzothiazol-2-ylamino)-1H- |
| | benzoimidazole-5-carboxylic acid piperidin-4-ylamide hydrochloride |
| 94 | 3-{[1-Methyl-2-(6-trifluoromethoxy-benzothiazol-2-ylamino)-1H- |
| | benzoimidazole-5-carbonyl]-amino}-piperidine-1-carboxylic acid tert-butyl ester |
| 95 | 1-Methyl-2-(6-trifluoromethoxy-benzothiazol-2-ylamino)-1H- |
| | benzoimidazole-5-carboxylic acid piperidin-3-ylamide hydrochloride |
| 96 | 1-Methyl-2-(6-trifluoromethoxy-benzothiazol-2-ylamino)-1H- |
| | benzoimidazole-5-carboxylic acid (thiazol-2-ylmethyl)-amide |
| 97 | 3-{[1-Methyl-2-(6-trifluoromethoxy-benzothiazol-2-ylamino)-1H- |
| | benzoimidazole-5-carbonyl]-aminol-propionic acid methyl ester |
| 98 | 3-{[2-(6-Trifluoromethoxy-benzothiazol-2-ylamino)-1-methyl-1H- |
| | benzimidazole-5-carbonyl]-aminol-propionic acid |
| 99 | 1-Methyl-2-(5-trifluoromethoxy-benzothiazol-2-ylamino)-1H- |
| | benzoimidazole-5-carboxylic acid methyl ester |
| 100 | 1-Methyl-2-(6-trifluoromethoxy-benzothiazol-2-ylamino)-1H- |
| | benzoimidazole-5-carboxylic acid (2-acetylamino-ethyl)-amide |
| 101 | 1-Methyl-2-(6-trifluoromethoxy-benzothiazol-2-ylamino)-1H- |
| | benzoimidazole-5-carboxylic acid (2-methylsulfanyl-ethyl)-amide |
| 102 | 1-Methyl-2-(6-trifluoromethoxy-benzothiazol-2-ylamino)-1H- |
| | benzoimidazole-5-carboxylic acid (2-methanesulfonyl-ethyl)-amide |
| 103 | (2-{[1-Methyl-2-(6-trifluoromethoxy-benzothiazol-2-ylamino)-1H- |
| | benzoimidazole-5-carbonyl]-aminol-ethyl)-carbamic acid tert-butyl ester |
| 104 | 1-Methyl-2-(6-trifluoromethoxy-benzothiazol-2-ylamino)-1H- |
| | benzoimidazole-5-carboxylic acid (2-amino-ethyl)-amide hydrochloride |
| 105 | 1-Methyl-2-(6-trifluoromethoxy-benzothiazol-2-ylamino)-1H- |
| | benzoimidazole-5-carboxylic acid (2-methylamino-ethyl)-amide |
| 106 | 1-Methyl-2-(6-trifluoromethoxy-benzothiazol-2-ylamino)-1H- |
| | benzimidazole-5-carboxylic acid trimethylhydrazide |
| 107 | 1-Methyl-2-(6-trifluoromethoxy-benzothiazol-2-ylamino)-1H- |
| | benzoimidazole-5-carboxylic acid (2-ethylsulfanyl-ethyl)-amide |
| 108 | 1-Methyl-2-(6-trifluoromethoxy-benzothiazol-2-ylamino)-1H- |
| | benzoimidazole-5-carboxylic acid (3-methylsulfanyl-propyl)-amide |
| 109 | 1-Methyl-2-(6-trifluoromethoxy-benzothiazol-2-ylamino)-1H- |
| | benzimidazole-5-carboxylic acid (2-ethanesulfonyl-ethyl)-amide |
| 110 | 1-Methyl-2-(6-trifluoromethoxy-benzothiazol-2-ylamino)-1H- |
| | benzimidazole-5-carboxylic acid (3-methanesulfonyl-propyl)-amide |
| 111 | 2-(5-Fluoro-benzothiazol-2-ylamino)-1-methyl-1H-benzoimidazole-5- |
| | carboxylic acid methyl ester |
| 112 | 2-(6-Fluoro-benzothiazol-2-ylamino)-1-methyl-1H-benzoimidazole-5- |
| | carboxylic acid methyl ester |
| 113 | 2-(6-Methanesulfonyl-benzothiazol-2-ylamino)-1-methyl-1H- |
| | benzoimidazole-5-carboxylic acid methyl ester |
| 114 | 1-Methyl-2-(6-methyl-benzothiazol-2-ylamino)-1H-benzoimidazole-5- |
| | carboxylic acid methyl ester |
| 115 | 1-Methyl-2-(5-trifluoromethoxy-benzothiazol-2-ylamino)-1H- |
| | benzimidazole-5-carboxylic acid methylamide |
| 116 | 1-Methyl-2-(5-trifluoromethoxy-benzothiazol-2-ylamino)-1H- |
| | benzimidazole-5-carboxylic acid (2-methoxy-ethyl)-amide |
| 117 | 2-(5-Fluoro-benzothiazol-2-ylamino)-1-methyl-1H-benzimidazole-5- |
| | carboxylic acid |
| 118 | 2-(6-Fluoro-benzothiazol-2-ylamino)-1-methyl-1H-benzimidazole-5- |
| | carboxylic acid |
| 119 | 2-(6-Methanesulfonyl-benzothiazol-2-ylamino)-1-methyl-1H- |
| | benzimidazole-5-carboxylic acid |
| 120 | 1-Methyl-2-(6-methyl-benzothiazol-2-ylamino)-1H-benzimidazole-5- |
| | carboxylic acid |
| 121 | 1-Methyl-2-(6-trifluoromethoxy-benzothiazol-2-ylamino)-1H- |
| | benzimidazole-5-carboxylic acid (1,1-dioxo-tetrahydro-1λ6-thiophen-3-yl)-amide |
| 122 | 2-(5-Fluoro-benzothiazol-2-ylamino)-1-methyl-1H-benzimidazole-5- |
| | carboxylic acid methylamide |
| 123 | 2-(5-Fluoro-benzothiazol-2-ylamino)-1-methyl-1H-benzimidazole-5- |
| | carboxylic acid (2-methoxy-ethyl)-amide |
| 124 | 2-(6-Fluoro-benzothiazol-2-ylamino)-1-methyl-1H-benzimidazole-5- |
| | carboxylic acid methylamide |
| 125 | 2-(6-Fluoro-benzothiazol-2-ylamino)-1-methyl-1H-benzimidazole-5- |
| | carboxylic acid (2-methoxy-ethyl)-amide |
| 126 | 2-(6-Methanesulfonyl-benzothiazol-2-ylamino)-1-methyl-1H- |
| | benzimidazole-5-carboxylic acid methylamide |
| 127 | 2-(6-Methanesulfonyl-benzothiazol-2-ylamino)-1-methyl-1H- |
| | benzimidazole-5-carboxylic acid (2-methoxy-ethyl)-amide |
| 128 | 2-(6-Methyl-benzothiazol-2-ylamino)-1-methyl-1H-benzimidazole-5- |
| | carboxylic acid methylamide |
| 129 | 2-(6-Methyl-benzothiazol-2-ylamino)-1-methyl-1H-benzimidazole-5- |
| | carboxylic acid (2-methoxy-ethyl)-amide |
| 130 | 2-(6-Methanesulfonyl-benzothiazol-2-ylamino)-1-methyl-1H- |
| | benzimidazole-5-carboxylic acid (2-methylsulfanyl-ethyl)-amide |
| 131 | 2-(6-Methanesulfonyl-benzothiazol-2-ylamino)-1-methyl-1H- |
| | benzimidazole-5-carboxylic acid (2-methylsulfonyl-ethyl)-amide |
| 132 | 1-Methyl-2-(6-trifluoromethylsulfanyl-benzothiazol-2-ylamino)-1H- |
| | benzoimidazole-5-carboxylic acid methyl ester |
| 133 | 2-(6-Chloro-benzothiazol-2-ylamino)-1-methyl-1H-benzimidazole-5- |
| | carboxylic acid dimethylcarbamoylmethyl-amide |
| 134 | 1-Methyl-2-(6-trifluoromethyl-benzothiazol-2-ylamino)-1H-benzimidazole- |
| | 5-carboxylic acid dimethylcarbamoylmethyl-amide |
| 135 | 1-Methyl-2-(6-trifluoromethoxy-benzothiazol-2-ylamino)-1H- |
| | benzimidazole-5-carboxylic acid (2-dimethylcarbamoyl-ethyl)-amide |
| 136 | 3-{[1-Methyl-2-(6-trifluoromethoxy-benzothiazol-2-ylamino)-1H- |
| | benzimidazole-5-carbonyl]-amino}-propionic acid tert-butyl ester |
| 137 | 1-Methyl-2-(6-trifluoromethoxy-benzothiazol-2-ylamino)-1H- |
| | benzimidazole-5-carboxylic acid [2-(4-methyl-piperazin-1-yl)-2-oxo-ethyl]-amide |
| 138 | 1-Methyl-2-(6-trifluoromethoxy-benzothiazol-2-ylamino)-1H- |
| | benzimidazole-5-carboxylic acid (2-morpholin-4-yl-2-oxo-ethyl)-amide |
| 139 | 1-Methyl-2-(6-trifluoromethoxy-benzothiazol-2-ylamino)-1H- |
| | benzimidazole-5-carboxylic acid methylcarbamoylmethyl-amide |
| 140 | 1-Methyl-2-(6-trifluoromethoxy-benzothiazol-2-ylamino)-1H- |
| | benzimidazole-5-carboxylic acid diethylcarbamoylmethyl-amide |
| 141 | 1-Methyl-2-(6-trifluoromethoxy-benzothiazol-2-ylamino)-1H- |
| | benzimidazole-5-carboxylic acid (2-oxo-2-pyrrolidin-1-yl-ethyl)-amide |
| 142 | 4-(2-{[1-Methyl-2-(6-trifluoromethoxy-benzothiazol-2-ylamino)-1H- |
| | benzimidazole-5-carbonyl]-amino}-acetyl)-piperazine-1-carboxylic acid |
| | tert-butyl ester |
| 143 | (S)-2-{[1-Methyl-2-(6-trifluoromethoxy-benzothiazol-2-ylamino)-1H- |
| | benzoimidazole-5-carbonyl]-amino}-propionic acid methyl ester |
| 144 | 1-{[1-Methyl-2-(6-trifluoromethoxy-benzothiazol-2-ylamino)-1H- |
| | benzimidazole-5-carbonyl]-amino}-cyclopropanecarboxylic acid ethyl ester |
| 145 | 2-Methyl-2-{[1-methyl-2-(6-trifluoromethoxy-benzothiazol-2-ylamino)-1H- |
| | benzimidazole-5-carbonyl]-amino}-propionic acid methyl ester |
| 146 | (S)-2-{[1-Methyl-2-(6-trifluoromethoxy-benzothiazol-2-ylamino)-1H- |
| | benzoimidazole-5-carbonyl]-aminol-propionic acid |
| 147 | 1-{[1-Methyl-2-(6-trifluoromethoxy-benzothiazol-2-ylamino)-1H- |
| | benzimidazole-5-carbonyl]-amino}-cyclopropanecarboxylic acid |
| 148 | 2-Methyl-2-{[1-methyl-2-(6-trifluoromethoxy-benzothiazol-2-ylamino)-1H- |
| | benzimidazole-5-carbonyl]-amino}-propionic acid |
| 149 | 1-Methyl-2-(6-trifluoromethoxy-benzothiazol-2-ylamino)-1H- |
| | benzoimidazole-5-carboxylic acid ((S)-1-dimethylcarbamoyl-ethyl)-amide |
| 150 | 1-Methyl-2-(6-trifluoromethoxy-benzothiazol-2-ylamino)-1H- |
| | benzimidazole-5-carboxylic acid (1-dimethylcarbamoyl-cyclopropyl)-amide |
| 151 | 1-Methyl-2-(6-trifluoromethoxy-benzothiazol-2-ylamino)-1H- |
| | benzimidazole-5-carboxylic acid (1-dimethylcarbamoyl-1-methyl-ethyl)-amide |
| 152 | 1-Methyl-2-(6-trifluoromethoxy-benzothiazol-2-ylamino)-1H- |
| | benzimidazole-5-carboxylic acid (2-oxo-2-piperazin-1-yl-ethyl)-amide |
| | hydrochloride |
| 153 | 1-Ethyl-2-(6-trifluoromethoxy-benzothiazol-2-ylamino)-1H- |
| | benzoimidazole-5-carboxylic acid methyl ester |
| 154 | 1-Ethyl-2-(6-trifluoromethoxy-benzothiazol-2-ylamino)-1H- |
| | benzoimidazole-5-carboxylic acid |
| 155 | 1-Ethyl-2-(6-trifluoromethoxy-benzothiazol-2-ylamino)-1H- |
| | benzoimidazole-5-carboxylic acid methylamide |
| 156 | 1-Ethyl-2-(6-trifluoromethoxy-benzothiazol-2-ylamino)-1H- |
| | benzoimidazole-5-carboxylic acid ethylamide |
| 157 | 1-Ethyl-2-(6-trifluoromethoxy-benzothiazol-2-ylamino)-1H- |
| | benzoimidazole-5-carboxylic acid (2-ethoxy-ethyl)-amide |
| 158 | 1-Isopropyl-2-(6-trifluoromethoxy-benzothiazol-2-ylamino)-1H |
| | benzoimidazole-5-carboxylic acid methyl ester |
| 159 | 1-Isopropyl-2-(6-trifluoromethoxy-benzothiazol-2-ylamino)-1H- |
| | benzoimidazole-5-carboxylic acid |
| 160 | 1-Isopropyl-2-(6-trifluoromethoxy-benzothiazol-2-ylamino)-1H- |
| | benzoimidazole-5-carboxylic acid methylamide |
| 161 | 1-Isopropyl-2-(6-trifluoromethoxy-benzothiazol-2-ylamino)-1H- |
| | benzoimidazole-5-carboxylic acid ethylamide |
| 162 | 1-Isobutyl-2-(6-trifluoromethoxy-benzothiazol-2-ylamino)-1H- |
| | benzoimidazole-5-carboxylic acid methyl ester |
| 163 | 1-Isobutyl-2-(6-trifluoromethoxy-benzothiazol-2-ylamino)-1H- |
| | benzoimidazole-5-carboxylic acid |
| 164 | 1-Isobutyl-2-(6-trifluoromethoxy-benzothiazol-2-ylamino)-1H- |
| | benzoimidazole-5-carboxylic acid methylamide |
| 165 | 1-Isobutyl-2-(6-trifluoromethoxy-benzothiazol-2-ylamino)-1H- |
| | benzoimidazole-5-carboxylic acid ethylamide |
| 166 | 1-(2-Methoxy-ethyl)-2-(6-trifluoromethoxy-benzothiazol-2-ylamino)-1H- |
| | benzoimidazole-5-carboxylic acid |
| 167 | 1-(2-Methoxy-ethyl)-2-(6-trifluoromethoxy-benzothiazol-2-ylamino)-1H- |
| | benzoimidazole-5-carboxylic acid methylamide |
| 168 | 1-(2-Methoxy-ethyl)-2-(6-trifluoromethoxy-benzothiazol-2-ylamino)-1H- |
| | benzoimidazole-5-carboxylic acid (2-methoxy-ethyl)-amide |
| 169 | 1-(2-Methoxy-ethyl)-2-(6-trifluoromethoxy-benzothiazol-2-ylamino)-1H- |
| | benzoimidazole-5-carboxylic acid (2-ethoxy-ethyl)-amide |
| 170 | 1-(2-Fluoro-ethyl)-2-(6-trifluoromethoxy-benzothiazol-2-ylamino)-1H- |
| | benzoimidazole-5-carboxylic acid methylamide |
| 171 | 1-(2-Fluoro-ethyl)-2-(6-trifluoromethoxy-benzothiazol-2-ylamino)-1H- |
| | benzoimidazole-5-carboxylic acid (2-methoxy-ethyl)-amide |
| 172 | 1-(2-Fluoro-ethyl)-2-(6-trifluoromethoxy-benzothiazol-2-ylamino)-1H- |
| | benzoimidazole-5-carboxylic acid (2-ethoxy-ethyl)-amide |
| 173 | 1-(2-Amino-ethyl)-2-(6-trifluoromethoxy-benzothiazol-2-ylamino)-1H- |
| | benzoimidazole-5-carboxylic acid methylamide hydrochloride |
| 174 | 2-(6-Chloro-benzothiazol-2-ylamino)-1-ethyl-1H-benzoimidazole-5- |
| | carboxylic acid methylamide |
| 175 | 2-(6-Chloro-benzothiazol-2-ylamino)-1-ethyl-1H-benzoimidazole-5- |
| | carboxylic acid ethylamide |
| 176 | 2-(6-Chloro-benzothiazol-2-ylamino)-1-ethyl-1H-benzoimidazole-5- |
| | carboxylic acid (2-fluoro-ethyl)-amide |
| 177 | 2-(6-Chloro-benzothiazol-2-ylamino)-1-ethyl-1H-benzoimidazole-5- |
| | carboxylic acid (2-methoxy-ethyl)-amide |
| 178 | 2-(6-Chloro-benzothiazol-2-ylamino)-1-ethyl-1H-benzoimidazole-5- |
| | carboxylic acid (2-methoxy-2-methyl-propyl)-amide |
| 179 | 2-(6-Chloro-benzothiazol-2-ylamino)-1-ethyl-1H-benzoimidazole-5- |
| | carboxylic acid (2-ethoxy-ethyl)-amide |
| 180 | 1-Ethyl-2-(6-trifluoromethyl-benzothiazol-2-ylamino)-1H-benzoimidazole- |
| | 5-carboxylic acid methylamide |
| 181 | 1-Ethyl-2-(6-trifluoromethyl-benzothiazol-2-ylamino)-1H-benzoimidazole- |
| | 5-carboxylic acid ethylamide |
| 182 | 1-Ethyl-2-(6-trifluoromethyl-benzothiazol-2-ylamino)-1H-benzoimidazole- |
| | 5-carboxylic acid (2-methoxy-ethyl)-amide |
| 183 | 1-Ethyl-2-(6-trifluoromethyl-benzothiazol-2-ylamino)-1H-benzoimidazole- |
| | 5-carboxylic acid (2-ethoxy-ethyl)-amide |
| 184 | 1-Ethyl-2-(6-trifluoromethyl-benzothiazol-2-ylamino)-1H-benzoimidazole- |
| | 5-carboxylic acid (2-methoxy-2-methyl-propyl)-amide |
| 185 | 1-Ethyl-2-(6-trifluoromethyl-benzothiazol-2-ylamino)-1H-benzoimidazole- |
| | 5-carboxylic acid (2-methylsulfanyl-ethyl)-amide |
| 186 | 1-Ethyl-2-(6-trifluoromethoxy-benzothiazol-2-ylamino)-1H- |
| | benzoimidazole-5-carboxylic acid dimethylcarbamoylmethyl-amide |
| 187 | 1-Ethyl-2-(6-trifluoromethyl-benzothiazol-2-ylamino)-1H-benzoimidazole- |
| | 5-carboxylic acid dimethylcarbamoylmethyl-amide |
| 188 | 1-(2-Methoxy-ethyl)-2-(6-trifluoromethoxy-benzothiazol-2-ylamino)-1H- |
| | benzoimidazole-5-carboxylic acid dimethylcarbamoylmethyl-amide |
| 189 | 1-(2-Methoxy-ethyl)-2-(6-trifluoromethoxy-benzothiazol-2-ylamino)-1H- |
| | benzoimidazole-5-carboxylic acid [2-(4-methyl-piperazin-1-yl)-2-oxo-ethyl]-amide |
| 190 | 1-Ethyl-2-(6-trifluoromethoxy-benzothiazol-2-ylamino)-1H- |
| | benzoimidazole-5-carboxylic acid [2-(4-methyl-piperazin-1-yl)-2-oxo-ethyl]-amide |
| 191 | 1-Ethyl-2-[6-(pyridin-3-yloxy)-benzothiazol-2-ylamino]-1H- |
| | benzoimidazole-5-carboxylic acid (2-methoxy-ethyl)-amide |
| 192 | 1-Methyl-2-(6-trifluoromethoxy-benzothiazol-2-ylamino)-1H- |
| | benzoimidazole-5-carboxylic acid [2-(4-hydroxy-piperidin-1-yl)-ethyl]-amide |
| 193 | 1-Methyl-2-(6-trifluoromethoxy-benzothiazol-2-ylamino)-1H- |
| | benzoimidazole-5-carboxylic acid [2-(3-hydroxy-piperidin-1-yl)-ethyl]-amide |
| 194 | 1-Methyl-2-(6-trifluoromethoxy-benzothiazol-2-ylamino)-1H- |
| | benzoimidazole-5-carbonitrile |
| 195 | 1-Methyl-2-(6-trifluoromethoxy-benzothiazol-2-ylamino)-1H- |
| | benzoimidazole-6-carbonitrile |
| 196 | [5-(1H-Imidazol-2-yl)-1-methyl-1H-benzimidazol-2-yl]-(6- |
| | trifluoromethoxy-benzothiazol-2-yl)-amine |
| 197 | [1-Methyl-6-(1H-1,2,4-triazol-3-yl)-1H-benzimidazol-2-yl]-(6- |
| | trifluoromethoxy-benzothiazol-2-yl)-amine |
| 198 | [1-Methyl-6-(5-methyl-1H-1,2,4-triazol-3-yl)-1H-benzimidazol-2-yl]-(5- |
| | trifluoromethoxy-benzothiazol-2-yl)-amine |
| 199 | (1-Ethyl-5-trifluoromethanesulfonyl-1H-benzoimidazol-2-yl)-(6- |
| | trifluoromethoxy-benzothiazol-2-yl)-amine |
| 200 | 1-[1-Methyl-2-(6-trifluoromethoxy-benzothiazol-2-ylamino)-1H- |
| | benzoimidazol-5-yl]-ethanone |
| 201 | (5-Methanesulfonyl-1-methyl-1H-benzoimidazol-2-yl)-(6-trifluoromethoxy- |
| | benzothiazol-2-yl)-amine |
| 202 | 2-[1-Methyl-2-(6-trifluoromethoxy-benzothiazol-2-ylamino)-1H- |
| | benzoimidazol-6-yl]-acetamide |
| 203 | 2-(6-Chloro-benzothiazol-2-ylamino)-1-methyl-1H-benzoimidazole-5- |
| | carboxylic acid ((R)-2-hydroxy-propyl)-amide |
| 204 | 2-(6-Chloro-benzothiazol-2-ylamino)-1-methyl-1H-benzoimidazole-5- |
| | carboxylic acid ((S)-2-hydroxy-propyl)-amide |
| 205 | 1-Methyl-2-(6-trifluoromethyl-benzothiazol-2-ylamino)-1H- |
| | benzoimidazole-5-carboxylic acid ((R)-2-hydroxy-propyl)-amide |
| 206 | 1-Methyl-2-(6-trifluoromethyl-benzothiazol-2-ylamino)-1H- |
| | benzoimidazole-5-carboxylic acid ((S)-2-hydroxy-propyl)-amide |
| 207 | 2-(6-Chloro-benzothiazol-2-ylamino)-1-methyl-1H-benzoimidazole-5- |
| | carboxylic acid (2-methoxy-2-methyl-propyl)-amide |
| 208 | 1-Methyl-2-(6-trifluoromethyl-benzothiazol-2-ylamino)-1H- |
| | benzoimidazole-5-carboxylic acid (2-methoxy-2-methyl-propyl)-amide |
| 209 | 2-(6-Chloro-benzothiazol-2-ylamino)-1-methyl-1H-benzoimidazole-5- |
| | carboxylic acid (2-fluoro-ethyl)-amide |
| 210 | 1-Methyl-2-(6-trifluoromethyl-benzothiazol-2-ylamino)-1H- |
| | benzoimidazole-5-carboxylic acid (2-fluoro-ethyl)-amide |
| 211 | 1-Methyl-2-(6-trifluoromethoxy-benzothiazol-2-ylamino)-1H- |
| | benzoimidazole-5-carboxylic acid cyanomethyl-amide |
| 212 | 1-Methyl-2-(6-trifluoromethoxy-benzothiazol-2-ylamino)-1H- |
| | benzoimidazole-5-carboxylic acid (2-cyano-ethyl)-amide |
| 213 | 2-(6-Chloro-benzothiazol-2-ylamino)-1-methyl-1H-benzoimidazole-5- |
| | carboxylic acid (2-cyano-ethyl)-amide |
| 214 | 2-(6-Chloro-benzothiazol-2-ylamino)-1-methyl-1H-benzoimidazole-5- |
| | carboxylic acid (3-hydroxy-propyl)-amide |
| 215 | 2-(6-Chloro-benzothiazol-2-ylamino)-1-methyl-1H-benzoimidazole-5- |
| | carboxylic acid (3-hydroxy-butyl)-amide |
| 216 | 1-Methyl-2-(6-trifluoromethyl-benzothiazol-2-ylamino)-1H- |
| | benzoimidazole-5-carboxylic acid (3-hydroxy-butyl)-amide |
| 217 | 1-Methyl-2-(6-trifluoromethoxy-benzothiazol-2-ylamino)-1H- |
| | benzoimidazole-5-carboxylic acid (3-hydroxy-2,2-dimethyl-propyl)-amide |
| 218 | 2-(6-Chloro-benzothiazol-2-ylamino)-1-methyl-1H-benzoimidazole-5- |
| | carboxylic acid (3-hydroxy-2,2-dimethyl-propyl)-amide |
| 219 | 1-Methyl-2-(6-trifluoromethoxy-benzothiazol-2-ylamino)-1H- |
| | benzoimidazole-5-carboxylic acid (4-hydroxy-butyl)-amide |
| 220 | 2-(6-Chloro-1H-benzoimidazol-2-ylamino)-1-methyl-1H-benzoimidazole-5- |
| | carboxylic acid (4-hydroxy-butyl)-amide |
| 221 | 1-Methyl-2-(6-trifluoromethyl-benzothiazol-2-ylamino)-1H- |
| | benzoimidazole-5-carboxylic acid (4-hydroxy-butyl)-amide |
| 222 | 6-Fluoro-1-methyl-2-(6-trifluoromethoxy-benzothiazol-2-ylamino)-1H- |
| | benzoimidazole-5-carboxylic acid (4-hydroxy-butyl)-amide |
| 223 | 1-Methyl-2-(6-trifluoromethoxy-benzothiazol-2-ylamino)-1H- |
| | benzoimidazole-5-carboxylic acid ((R)-4-hydroxy-3-methyl-butyl)-amide |
| 224 | 2-(6-Chloro-benzothiazol-2-ylamino)-1-methyl-1H-benzoimidazole-5- |
| | carboxylic acid ((R)-4-hydroxy-3-methyl-butyl)-amide |
| 225 | 2-(6-Chloro-benzothiazol-2-ylamino)-1-methyl-1H-benzoimidazole-5- |
| | carboxylic acid (trans-4-hydroxy-cyclohexyl)-amide |
| 226 | 1-Methyl-2-(6-trifluoromethoxy-benzothiazol-2-ylamino)-1H- |
| | benzoimidazole-5-carboxylic acid (5-hydroxy-pentyl)-amide |
| 227 | 2-(6-Chloro-benzothiazol-2-ylamino)-1-methyl-1H-benzoimidazole-5- |
| | carboxylic acid (5-hydroxy-pentyl)-amide |
| 228 | 1-Methyl-2-(6-trifluoromethyl-benzothiazol-2-ylamino)-1H- |
| | benzoimidazole-5-carboxylic acid (5-hydroxy-pentyl)-amide |
| 229 | 1-Methyl-2-(6-trifluoromethoxy-benzothiazol-2-ylamino)-1H- |
| | benzoimidazole-5-carboxylic acid (5-hydroxy-4,4-dimethyl-pentyl)-amide |
| 230 | 1-Methyl-2-(6-trifluoromethoxy-benzothiazol-2-ylamino)-1H- |
| | benzoimidazole-5-carboxylic acid 4-hydroxy-benzylamide |
| 231 | 1-Methyl-2-(6-trifluoromethoxy-benzothiazol-2-ylamino)-1H- |
| | benzoimidazole-5-carboxylic acid 3-hydroxy-4-methoxy-benzylamide |
| 232 | 1-Methyl-2-(6-trifluoromethoxy-benzothiazol-2-ylamino)-1H- |
| | benzoimidazole-5-carboxylic acid (trans-4-hydroxy-cyclohexylmethyl)-amide |
| 233 | 2-(6-Chloro-benzothiazol-2-ylamino)-1-methyl-1H-benzoimidazole-5- |
| | carboxylic acid (trans-4-hydroxy-cyclohexylmethyl)-amide |
| 234 | 1-Methyl-2-(6-trifluoromethyl-benzothiazol-2-ylamino)-1H- |
| | benzoimidazole-5-carboxylic acid (trans-4-hydroxy-cyclohexylmethyl)-amide |
| 235 | 2-(6-Chloro-benzothiazol-2-ylamino)-1-methyl-1H-benzoimidazole-5- |
| | carboxylic acid [2-(2-hydroxy-ethoxy)-ethyl]-amide |
| 236 | 1-Methyl-2-(6-trifluoromethyl-benzothiazol-2-ylamino)-1H- |
| | benzoimidazole-5-carboxylic acid [2-(2-hydroxy-ethoxy)-ethyl]-amide |
| 237 | 6-Fluoro-1-methyl-2-(6-trifluoromethoxy-benzothiazol-2-ylamino)-1H- |
| | benzoimidazole-5-carboxylic acid [2-(2-hydroxy-ethoxy)-ethyl]-amide |
| 238 | 3-Methyl-2-(6-trifluoromethoxy-benzothiazol-2-ylamino)-3H-imidazo[4,5- |
| | b]pyridine-6-carboxylic acid [2-(2-hydroxy-ethoxy)-ethyl]-amide |
| 239 | 1-(2-Methylamino-ethyl)-2-(6-trifluoromethoxy-benzothiazol-2 ylamino)- |
| | 1H-benzoimidazole-5-carboxylic acid[2-(2-hydroxy-ethoxy)-ethyl]-amide |
| | hydrochloride |
| 240 | 2-(6-Chloro-benzothiazol-2-ylamino)-1-(2-methylamino-ethyl)-1H- |
| | benzoimidazole-5-carboxylic acid [2-(2-hydroxy-ethoxy)-ethyl]-amide |
| | hydrochloride |
| 241 | 1-(2-Methoxy-ethyl)-2-(6-trifluoromethoxy-benzothiazol-2-ylamino)-1H- |
| | benzoimidazole-5-carboxylic acid [2-(2-hydroxy-ethoxy)-ethyl]-amide |
| 242 | 1-Methyl-2-(6-trifluoromethoxy-benzothiazol-2-ylamino)-1H- |
| | benzoimidazole-5-carboxylic acid [2-((R)-2-hydroxy-1-methyl-ethoxy)- |
| | ethyl]-amide |
| 243 | 2-(6-Chloro-benzothiazol-2-ylamino)-1-methyl-1H-benzoimidazole-5- |
| | carboxylic acid [2-((R)-2-hydroxy-1-methyl-ethoxy)-ethyl]-amide |
| 244 | 1-Methyl-2-(6-trifluoromethoxy-benzothiazol-2-ylamino)-1H- |
| | benzoimidazole-5-carboxylic acid [2-(2-hydroxy-propoxy)-ethyl]-amide |
| 245 | 1-Methyl-2-(6-trifluoromethoxy-benzothiazol-2-ylamino)-1H- |
| | benzoimidazole-5-carboxylic acid [2-(2-hydroxy-2-methyl-propoxy)-ethyl]-amide |
| 246 | 1-Methyl-2-(6-trifluoromethoxy-benzothiazol-2-ylamino)-1H- |
| | benzoimidazole-5-carboxylic acid [2-(3-hydroxy-propoxy)-ethyl]-amide |
| 247 | 1-Methyl-2-(6-trifluoromethoxy-benzothiazol-2-ylamino)-1H- |
| | benzoimidazole-5-carboxylic acid [2-(3-fluoro-propoxy)-ethyl]-amide |
| 248 | 2-(6-Chloro-benzothiazol-2-ylamino)-1-methyl-1H-benzoimidazole-5- |
| | carboxylic acid [2-(3-hydroxy-propoxy)-ethyl]-amide |
| 249 | 2-(6-Chloro-benzothiazol-2-ylamino)-1-methyl-1H-benzoimidazole-5- |
| | carboxylic acid [2-(3-fluoro-propoxy)-ethyl]-amide |
| 250 | 1-Methyl-2-(6-trifluoromethoxy-benzothiazol-2-ylamino)-1H- |
| | benzoimidazole-5-carboxylic acid [3-(2-hydroxy-ethoxy)-propyl]-amide |
| 251 | 1-Methyl-2-(6-trifluoromethoxy-benzothiazol-2-ylamino)-1H- |
| | benzoimidazole-5-carboxylic acid [2-(4-hydroxy-phenyl)-ethyl]-amide |
| 252 | 1-Methyl-2-(6-trifluoromethoxy-benzothiazol-2-ylamino)-1H- |
| | benzoimidazole-5-carboxylic acid [2-(3-hydroxy-phenyl)-ethyl]-amide |
| 253 | 1-Methyl-2-(6-trifluoromethoxy-benzothiazol-2-ylamino)-1H- |
| | benzoimidazole-5-carboxylic acid [2-(4-hydroxy-cyclohexyl)-ethyl]-amide |
| 254 | 1-Methyl-2-(6-trifluoromethoxy-benzothiazol-2-ylamino)-1H- |
| | benzoimidazole-5-carboxylic acid (trans-4-hydroxymethyl- |
| | cyclohexylmethyl)-amide |
| 255 | 2-(6-Chloro-benzothiazol-2-ylamino)-1-methyl-1H-benzoimidazole-5- |
| | carboxylic acid (trans-4-hydroxymethyl-cyclohexylmethyl)-amide |
| 256 | 1-Methyl-2-(6-trifluoromethoxy-benzothiazol-2-ylamino)-1H- |
| | benzoimidazole-5-carboxylic acid {2-[2-(2-hydroxy-ethoxy)-ethoxy]-ethyl}-amide |
| 257 | 1-Methyl-2-(6-trifluoromethyl-benzothiazol-2-ylamino)-1H- |
| | benzoimidazole-5-carboxylic acid [2-(2-fluoro-ethoxy)-ethyl]-amide |
| 258 | 1-Methyl-2-(6-trifluoromethoxy-benzothiazol-2-ylamino)-1H- |
| | benzoimidazole-5-carboxylic acid [2-(2,2-difluoro-ethoxy)-ethyl]-amide |
| 259 | 2-(6-Chloro-benzothiazol-2-ylamino)-1-methyl-1H-benzoimidazole-5- |
| | carboxylic acid [2-(2,2-difluoro-ethoxy)-ethyl]-amide |
| 260 | 1-Methyl-2-(6-trifluoromethoxy-benzothiazol-2-ylamino)-1H- |
| | benzoimidazole-5-carboxylic acid [2-(2-methoxy-ethoxy)-ethyl]-amide |
| 261 | 2-(6-Chloro-benzothiazol-2-ylamino)-1-methyl-1H-benzoimidazole-5- |
| | carboxylic acid [2-(2-methoxy-ethoxy)-ethyl]-amide |
| 262 | 1-Methyl-2-(6-trifluoromethyl-benzothiazol-2-ylamino)-1H- |
| | benzoimidazole-5-carboxylic acid [2-(2-methoxy-ethoxy)-ethyl]-amide |
| 263 | 1-Methyl-2-(6-trifluoromethoxy-benzothiazol-2-ylamino)-1H- |
| | benzoimidazole-5-carboxylic acid [2-(tetrahydro-pyran-2-yl)-ethyl]-amide |
| 264 | 1-Methyl-2-(6-trifluoromethoxy-benzothiazol-2-ylamino)-1H- |
| | benzoimidazole-5-carboxylic acid [2-(tetrahydro-pyran-4-yl)-ethyl]-amide |
| 265 | 1-Methyl-2-(6-trifluoromethoxy-benzothiazol-2-ylamino)-1H- |
| | benzoimidazole-5-carboxylic acid [2-(2-cyano-ethoxy)-ethyl]-amide |
| 266 | 2-(6-Chloro-benzothiazol-2-ylamino)-1-methyl-1H-benzoimidazole-5- |
| | carboxylic acid [2-(2-cyano-ethoxy)-ethyl]-amide |
| 267 | 1-Methyl-2-(6-trifluoromethoxy-benzothiazol-2-ylamino)-1H- |
| | benzoimidazole-5-carboxylic acid (2-carbamoylmethoxy-ethyl)-amide |
| 268 | 1-Methyl-2-(6-trifluoromethoxy-benzothiazol-2-ylamino)-1H |
| | benzoimidazole-5-carboxylic acid [2-(2-amino-ethoxy)-ethyl]-amide |
| 269 | 2-(6-Chloro-benzothiazol-2-ylamino)-1-methyl-1H-benzoimidazole-5- |
| | carboxylic acid [2-(2-amino-ethoxy)-ethyl]-amide |
| 270 | 2-(4-Chloro-benzothiazol-2-ylamino)-1-methyl-1H benzoimidazole-5- |
| | carboxylic acid [2-(2-amino-ethoxy)-ethyl]-amide |
| 271 | 1-Methyl-2-(6-trifluoromethoxy-benzothiazol-2-ylamino)-1H- |
| | benzoimidazole-5-carboxylic acid [2-(2-methylamino-ethoxy)-ethyl]-amide |
| | hydrochloride |
| 272 | 2-(6-Chloro-benzothiazol-2-ylamino)-1-methyl-1H-benzoimidazole-5- |
| | carboxylic acid [2-(2-methylamino-ethoxy)-ethyl]-amide hydrochloride |
| 273 | 1-Methyl-2-(6-trifluoromethoxy-benzothiazol-2-ylamino)-1H- |
| | benzoimidazole-5-carboxylic acid [2-(2-dimethylamino-ethoxy)-ethyl]-amide |
| 274 | 2-(6-Chloro-benzothiazol-2-ylamino)-1-methyl-1H-benzoimidazole-5- |
| | carboxylic acid [2-(2-dimethylamino-ethoxy)-ethyl]-amide |
| 275 | 1-Methyl-2-(6-trifluoromethoxy-benzothiazol-2-ylamino)-1H- |
| | benzoimidazole-5-carboxylic acid [2-(2-acetylamino-ethoxy)-ethyl]-amide |
| 276 | 1-Methyl-2-(6-trifluoromethoxy-benzothiazol-2-ylamino)-1H- |
| | benzoimidazole-5-carboxylic acid [2-(2-methanesulfonylamino-ethoxy)- |
| | ethyl]-amide |
| 277 | 2-(6-Chloro-benzothiazol-2-ylamino)-1-methyl-1H-benzoimidazole-5- |
| | carboxylic acid (2-ethanesulfonyl-ethyl)-amide |
| 278 | 2-(6-Chloro-benzothiazol-2-ylamino)-1-methyl-1H-benzoimidazole-5- |
| | carboxylic acid [2-(2-hydroxy-ethanesulfonyl)-ethyl]-amide |
| 279 | 2-(6-Chloro-benzothiazol-2-ylamino)-1-methyl-1H-benzoimidazole-5- |
| | carboxylic acid [2-(2-fluoro-ethylamino)-ethyl]-amide hydrochloride |
| 280 | 1-Methyl-2-(6-trifluoromethoxy-benzothiazol-2-ylamino)-1H- |
| | benzoimidazole-5-carboxylic acid ((S)-2,3-dihydroxy-propyl)-amide |
| 281 | 1-Methyl-2-(6-trifluoromethoxy-benzothiazol-2-ylamino)-1H- |
| | benzoimidazole-5-carboxylic acid ((R)-2,3-dihydroxy-propyl)-amide |
| 282 | 1-Methyl-2-(6-trifluoromethoxy-benzothiazol-2-ylamino)-1H- |
| | benzoimidazole-5-carboxylic acid ((1R,2S,3R,4R)-2,3-dihydroxy-4- |
| | hydroxymethyl-cyclopentyl)-amide |
| 283 | 1-Methyl-2-(6-trifluoromethoxy-benzothiazol-2-ylamino)-1H- |
| | benzoimidazole-5-carboxylic acid ((2S,3R,4R,5S,6R)-2,4,5-trihydroxy-6- |
| | hydroxymethyl-tetrahydro-pyran-3-yl)-amide |
| 284 | 1-Methyl-2-(6-trifluoromethoxy-benzothiazol-2-ylamino)-1H- |
| | benzoimidazole-5-carboxylic acid bis-(2-hydroxy-ethyl)-amide |
| 285 | 3-Methyl-2-(6-trifluoromethoxy-benzothiazol-2-ylamino)-3H- |
| | benzoimidazole-5-carboxylic acid (4-hydroxy-butyl)-amide |
| 286 | 3-Methyl-2-(6-trifluoromethoxy-benzothiazol-2-ylamino)-3H- |
| | benzoimidazole-5-carboxylic acid [2-(2-hydroxy-ethoxy)-ethyl]-amide |
| 287 | 2-(6-Chloro-benzothiazol-2-ylamino)-3-methyl-3H-benzoimidazole-5- |
| | carboxylic acid (4-hydroxy-butyl)-amide |
| 288 | 2-(6-Chloro-benzothiazol-2-ylamino)-3-methyl-3H-benzoimidazole-5- |
| | carboxylic acid [2-(2-hydroxy-ethoxy)-ethyl]-amide |
| 289 | 1-Methyl-2-(6-trifluoromethoxy-benzothiazol-2-ylamino)-1H- |
| | benzoimidazole-5-carboxylic acid pyrrolidin-3-ylamide hydrochloride |
| 290 | 2-(6-Chloro-benzothiazol-2-ylamino)-1-methyl-1H-benzoimidazole-5- |
| | carboxylic acid (R)-pyrrolidin-3-ylamide hydrochloride |
| 291 | 2-(6-Chloro-benzothiazol-2-ylamino)-1-methyl-1H-benzoimidazole-5- |
| | carboxylic acid (S)-pyrrolidin-3-ylamide hydrochloride |
| 292 | 2-(6-Chloro-benzothiazol-2-ylamino)-1-methyl-1H-benzoimidazole-5- |
| | carboxylic acid [(R)-1-(2-hydroxy-ethyl)-pyrrolidin-3-yl]-amide |
| 293 | 2-(6-Chloro-benzothiazol-2-ylamino)-1-methyl-1H-benzoimidazole-5- |
| | carboxylic acid [(S)-1-(2-hydroxy-ethyl)-pyrrolidin-3-yl]-amide |
| 294 | 1-Methyl-2-(6-trifluoromethoxy-benzothiazol-2-ylamino)-1H- |
| | benzoimidazole-5-carboxylic acid [1-((R)-2-hydroxy-propyl)-pyrrolidin-3- |
| | yl]-amide |
| 295 | 2-(6-Chloro-benzothiazol-2-ylamino)-1-methyl-1H-benzoimidazole-5- |
| | carboxylic acid [(R)-1-((S)-2-hydroxy-propionyl)-pyrrolidin-3-yl]-amide |
| 296 | 2-(6-Chloro-benzothiazol-2-ylamino)-1-methyl-1H-benzoimidazole-5- |
| | carboxylic acid [(R)-1-((R)-2-hydroxy-propyl)-pyrrolidin-3-yl]-amide |
| 297 | 1-Methyl-2-(6-trifluoromethyl-benzothiazol-2-ylamino)-1H- |
| | benzoimidazole-5-carboxylic acid [(R)-1-((R)-2-hydroxy-propyl)-pyrrolidin- |
| | 3-yl]-amide |
| 298 | 1-Methyl-2-(6-trifluoromethoxy-benzothiazol-2-ylamino)-1H- |
| | benzoimidazole-5-carboxylic acid [1-(2-hydroxy-2-methyl-propyl)- |
| | pyrrolidin-3-yl]-amide |
| 299 | 3-(3-{[2-(6-Chloro-benzothiazol-2-ylamino)-1-methyl-1H-benzoimidazole-5- |
| | carbonyl]-amino}-pyrrolidin-1-yl)-propionic acid |
| 300 | 2-(6-Chloro-benzothiazol-2-ylamino)-1-methyl-1H-benzoimidazole-5- |
| | carboxylic acid [1-(2-methanesulfonylamino-ethyl)-pyrrolidin-3-yl]-amide |
| 301 | 2-(6-Chloro-benzothiazol-2-ylamino)-1-(2-methoxy-ethyl)-1H- |
| | benzoimidazole-5-carboxylic acid [1-(2-hydroxy-ethyl)-piperidin-4-yl]-amide |
| 302 | 2-(6-Chloro-benzothiazol-2-ylamino)-1-methyl-1H-benzoimidazole-5- |
| | carboxylic acid (piperidin-4-ylmethyl)-amide hydrochloride |
| 303 | 2-(6-Chloro-benzothiazol-2-ylamino)-1-methyl-1H-benzoimidazole-5- |
| | carboxylic acid [1-(2-hydroxy-ethyl)-piperidin-4-ylmethyl]-amide |
| 304 | 2-(6-Chloro-benzothiazol-2-ylamino)-1-(2-methoxy-ethyl)-1H- |
| | benzoimidazole-5-carboxylic acid [1-(2-hydroxy-ethyl)-piperidin-4- |
| | ylmethyl]-amide |
| 305 | [4-(2-Hydroxy-ethyl)-piperazin-1-yl]-[1-methyl-2-(6-trifluoromethoxy- |
| | benzothiazol-2-ylamino)-1H-benzoimidazol-5-yl]-methanone |
| 306 | [2-(6-Chloro-benzothiazol-2-ylamino)-1-methyl-1H-benzoimidazol-5-yl]-[4- |
| | (2-hydroxy-ethyl)-piperazin-1-yl]-methanone |
| 307 | [4-(3-Hydroxy-propyl)-piperidin-1-yl]-[1-methyl-2-(6-trifluoromethoxy- |
| | benzothiazol-2-ylamino)-1H benzoimidazol-5-yl]-methanone |
| 308 | 2-(6-Chloro-benzothiazol-2-ylamino)-1-methyl-1H-benzoimidazole-5- |
| | carboxylic acid [(R)-1-(2-dimethylamino-acetyl)-pyrrolidin-3-yl]-amide |
| 309 | 2-(6-Chloro-benzothiazol-2-ylamino)-1-methyl-1H-benzoimidazole-5- |
| | carboxylic acid (R)-piperidin-3-ylamide hydrochloride |
| 310 | 2-(6-Chloro-benzothiazol-2-ylamino)-1-methyl-1H-benzoimidazole-5- |
| | carboxylic acid (S)-piperidin-3-ylamide hydrochloride |
| 311 | 2-(6-Chloro-benzothiazol-2-ylamino)-1-methyl-1H-benzoimidazole-5- |
| | carboxylic acid [(R)-1-(2-dimethylamino-acetyl)-piperidin-3-yl]-amide |
| 312 | 2-(6-Chloro-benzothiazol-2-ylamino)-1-methyl-1H-benzoimidazole-5- |
| | carboxylic acid [(S)-1-(2-dimethylamino-acetyl)-piperidin-3-yl]-amide |
| 313 | 2-(6-Chloro-benzothiazol-2-ylamino)-1-methyl-1H-benzoimidazole-5- |
| | carboxylic acid [1-(2-dimethylamino-acetyl)-piperidin-4-yl]-amide |
| 314 | 2-(6-Chloro-benzothiazol-2-ylamino)-1-methyl-1H-benzoimidazole-5- |
| | carboxylic acid [1-(2-dimethylamino-acetyl)-piperidin-4-ylmethyl]-amide |
| 315 | 2-(6-Chloro-benzothiazol-2-ylamino)-1-(2-methoxy-ethyl)-1H- |
| | benzoimidazole-5-carboxylic acid [1-(2-dimethylamino-acetyl)-piperidin-4- |
| | ylmethyl]-amide |
| 316 | 2-(6-Chloro-benzothiazol-2-ylamino)-1-methyl-1H benzoimidazole-5- |
| | carboxylic acid ((R)-1-methyl-pyrrolidin-3-yl)-amide |
| 317 | 2-(6-Chloro-benzothiazol-2 ylamino)-1-methyl-1H-benzoimidazole-5- |
| | carboxylic acid ((S)-1-methyl-pyrrolidin-3-yl)-amide |
| 318 | 2-(6-Chloro-benzothiazol-2-ylamino)-1-methyl-1H-benzoimidazole-5- |
| | carboxylic acid (1-methyl-piperidin-2-ylmethyl)-amide |
| 319 | 2-(6-Chloro-benzothiazol-2-ylamino)-1-methyl-1H-benzoimidazole-5- |
| | carboxylic acid (1-methyl-piperidin-4-yl)-amide |
| 320 | 2-(6-Chloro-benzothiazol-2-ylamino)-1-methyl-1H-benzoimidazole-5- |
| | carboxylic acid (1-methanesulfonyl-piperidin-4-yl)-amide |
| 321 | 2-(6-Chloro-benzothiazol-2-ylamino)-1-methyl-1H-benzoimidazole-5- |
| | carboxylic acid morpholin-4-ylamide |
| 322 | 1-Methyl-2-(6-trifluoromethoxy-benzothiazol-2-ylamino)-1H- |
| | benzoimidazole-5-carboxylic acid (2-methanesulfonylamino-ethyl)-amide |
| 323 | 1-Methyl-2-(6-trifluoromethoxy-benzothiazol-2-ylamino)-1H- |
| | benzoimidazole-5-carboxylic acid [2-(2-dimethylamino-acetylamino)- |
| | ethyl]-amide |
| 324 | 2-(6-Chloro-benzothiazol-2-ylamino)-1-methyl-1H-benzoimidazole-5- |
| | carboxylic acid [2-(2-dimethylamino-acetylamino)-ethyl]-amide |
| 325 | 1-Methyl-2-(6-trifluoromethoxy-benzothiazol-2-ylamino)-1H- |
| | benzoimidazole-5-carboxylic acid [2-(2-hydroxy-acetylamino)-ethyl]-amide |
| | 1-Methyl-2-(6-trifluoromethoxy-benzothiazol-2-ylamino)-1H- |
| 326 | benzoimidazole-5-carboxylic acid [2-((S)-2-hydroxy-propionylamino)- |
| | ethyl]-amide |
| 327 | 1-Methyl-2-(6-trifluoromethoxy-benzothiazol-2-ylamino)-1H- |
| | benzoimidazole-5-carboxylic acid (2-imidazol-1-yl-ethyl)-amide |
| 328 | 1-Methyl-2-(6-trifluoromethoxy-benzothiazol-2-ylamino)-1H- |
| | benzoimidazole-5-carboxylic acid (2-pyrazol-1-yl-ethyl)-amide |
| 329 | 1-Methyl-2-(6-trifluoromethoxy-benzothiazol-2-ylamino)-1H- |
| | benzoimidazole-5-carboxylic acid [2-(2-oxo-pyrrolidin-1-yl)-ethyl]-amide |
| 330 | 1-Methyl-2-(6-trifluoromethoxy-benzothiazol-2-ylamino)-1H- |
| | benzoimidazole-5-carboxylic acid [2-(2-oxo-imidazolidin-1-yl)-ethyl]-amide |
| 331 | 2-(6-Chloro-benzothiazol-2-ylamino)-1-methyl-1H-benzoimidazole-5- |
| | carboxylic acid [2-(3-oxo-piperazin-1-yl)-ethyl]-amide |
| 332 | 2-(6-Chloro-benzothiazol-2-ylamino)-1-(2-methoxy-ethyl)-1H- |
| | benzoimidazole-5-carboxylic acid [2-(3-oxo-piperazin-1-yl)-ethyl]-amide |
| 333 | 1-Methyl-2-(6-trifluoromethoxy-benzothiazol-2-ylamino)-1H- |
| | benzoimidazole-5-carboxylic acid (2-piperidin-1-yl-ethyl)-amide |
| 334 | 2-(6-Chloro-benzothiazol-2-ylamino)-1-methyl-1H-benzoimidazole-5- |
| | carboxylic acid (2-piperidin-1-yl-ethyl)-amide |
| 335 | 2-(6-Chloro-benzothiazol-2-ylamino)-1-methyl-1H-benzoimidazole-5- |
| | carboxylic acid [2-(4,4-difluoro-piperidin-1-yl)-ethyl]-amide |
| 336 | 2-(6-Chloro-benzothiazol-2-ylamino)-1-methyl-1H-benzoimidazole-5- |
| | carboxylic acid [2-(3,3-difluoro-piperidin-1-yl)-ethyl]-amide |
| 337 | 1-Methyl-2-(6-trifluoromethoxy-benzothiazol-2-ylamino)-1H- |
| | benzoimidazole-5-carboxylic acid [2-(4-methyl-piperazin-1-yl)-ethyl]-amide |
| 338 | 2-(6-Chloro-benzothiazol-2-ylamino)-1-methyl-1H-benzoimidazole-5- |
| | carboxylic acid [2-(4-methyl-piperazin-1-yl)-ethyl]-amide |
| 339 | 2-(6-Chloro-benzothiazol-2-ylamino)-1-methyl-1H-benzoimidazole-5- |
| | carboxylic acid (2-piperazin-1-yl-ethyl)-amide hydrochloride |
| 340 | 2-(6-Chloro-benzothiazol-2-ylamino)-1-methyl-1H-benzoimidazole-5- |
| | carboxylic acid (2-morpholin-4-yl-ethyl)-amide |
| 341 | 1-Methyl-2-(6-trifluoromethyl-benzothiazol-2-ylamino)-1H- |
| | benzoimidazole-5-carboxylic acid (2-morpholin-4-yl-ethyl)-amide |
| 342 | 1-Methyl-2-(6-trifluoromethoxy-benzothiazol-2-ylamino)-1H- |
| | benzoimidazole-5-carboxylic acid [2-(1,1-dioxo-thiomorpholin-4-yl)-ethyl]-amide |
| 343 | 2-(6-Chloro-benzothiazol-2-ylamino)-1-methyl-1H-benzoimidazole-5- |
| | carboxylic acid (2-amino-ethyl)-amide hydrochloride |
| 344 | 1-Methyl-2-(6-trifluoromethoxy-benzothiazol-2-ylamino)-1H- |
| | benzoimidazole-5-carboxylic acid (3-amino-propyl)-amide hydrochloride |
| 345 | 2-(6-Chloro-benzothiazol-2-ylamino)-1-methyl-1H-benzoimidazole-5- |
| | carboxylic acid (3-amino-propyl)-amide hydrochloride |
| 346 | 1-Methyl-2-(6-trifluoromethoxy-benzothiazol-2-ylamino)-1H- |
| | benzoimidazole-5-carboxylic acid (4-amino-butyl)-amide hydrochloride |
| 347 | 2-(6-Chloro-benzothiazol-2-ylamino)-1-methyl-1H-benzoimidazole-5- |
| | carboxylic acid (4-amino-butyl)-amide hydrochloride |
| 348 | 1-Methyl-2-(6-trifluoromethoxy-benzothiazol-2-ylamino)-1H- |
| | benzoimidazole-5-carboxylic acid (3-dimethylamino-propyl)-amide |
| 349 | 2-(6-Chloro-benzothiazol-2-ylamino)-1-methyl-1H-benzoimidazole-5- |
| | carboxylic acid (3-dimethylamino-propyl)-amide |
| 350 | 1-Methyl-2-(6-trifluoromethyl-benzothiazol-2-ylamino)-1H- |
| | benzoimidazole-5-carboxylic acid (3-dimethylamino-propyl)-amide |
| 351 | 1-Methyl-2-(6-trifluoromethoxy-benzothiazol-2-ylamino)-1H- |
| | benzoimidazole-5-carboxylic acid (3-diethylamino-propyl)-amide |
| 352 | 2-(6-Chloro-benzothiazol-2-ylamino)-1-methyl-1H-benzoimidazole-5- |
| | carboxylic acid (3-diethylamino-propyl)-amide |
| 353 | 1-Methyl-2-(6-trifluoromethyl-benzothiazol-2-ylamino)-1H- |
| | benzoimidazole-5-carboxylic acid (3-diethylamino-propyl)-amide |
| 354 | 2-(6-Chloro-benzothiazol-2-ylamino)-1-methyl-1H-benzoimidazole-5- |
| | carboxylic acid (3-pyrrolidin-1-yl-propyl)-amide |
| 355 | 1-Methyl-2-(6-trifluoromethoxy-benzothiazol-2-ylamino)-1H- |
| | benzoimidazole-5-carboxylic acid [3-(4-methyl-piperazin-1-yl)-propyl]-amide |
| 356 | 2-(6-Chloro-benzothiazol-2-ylamino)-1-methyl-1H-benzoimidazole-5- |
| | carboxylic acid [3-(4-methyl-piperazin-1-yl)-propyl]-amide |
| 357 | 1-Methyl-2-(6-trifluoromethyl-benzothiazol-2-ylamino)-1H- |
| | benzoimidazole-5-carboxylic acid [3-(4-methyl-piperazin-1-yl)-propyl]-amide |
| 358 | 1-Methyl-2-(6-trifluoromethoxy-benzothiazol-2-ylamino)-1H- |
| | benzoimidazole-5-carboxylic acid (3-morpholin-4-yl-propyl)-amide |
| 359 | 2-(6-Chloro-benzothiazol-2-ylamino)-1-methyl-1H-benzoimidazole-5- |
| | carboxylic acid (3-morpholin-4-yl-propyl)-amide |
| 360 | 1-Methyl-2-(6-trifluoromethoxy-benzothiazol-2-ylamino)-1H- |
| | benzoimidazole-5-carboxylic acid (4-diethylamino-butyl)-amide |
| 361 | 6-Diethylamino-1-methyl-2-(6-trifluoromethoxy-benzothiazol-2-ylamino)- |
| | 1H-benzoimidazole-5-carboxylic acid dimethylcarbamoylmethyl-amide |
| 362 | 6-Diethylamino-1-methyl-2-(6-trifluoromethoxy-benzothiazol-2-ylamino)- |
| | 1H-benzoimidazole-5-carboxylic acid (2-morpholin-4-yl-ethyl)-amide |
| 363 | 1-Methyl-2-(6-methyl-benzothiazol-2-ylamino)-1H-benzoimidazole-5- |
| | carboxylic acid dimethylcarbamoylmethyl-amide |
| 364 | 2-(6-Ethoxy-benzothiazol-2-ylamino)-1-methyl-1H-benzoimidazole-5- |
| | carboxylic acid dimethylcarbamoylmethyl-amide |
| 365 | 2-(6-Isopropyl-benzothiazol-2-ylamino)-1-methyl-1H-benzoimidazole-5- |
| | carboxylic acid dimethylcarbamoylmethyl-amide |
| 366 | 2-(6-Chloro-benzothiazol-2-ylamino)-1-(2-methoxy-ethyl)-1H- |
| | benzoimidazole-5-carboxylic acid dimethylcarbamoylmethyl-amide |
| 367 | 2-(6-Chloro-benzothiazol-2-ylamino)-1-(2-methylamino-ethyl)-1H- |
| | benzoimidazole-5-carboxylic acid dimethylcarbamoylmethyl-amide |
| | hydrochloride |
| 368 | 1-Methyl-2-(6-trifluoromethoxy-benzothiazol-2-ylamino)-1H- |
| | benzoimidazole-5-carboxylic acid ((S)-1-dimethylcarbamoyl-2-hydroxy- |
| | ethyl)-amide |
| 369 | 1-Methyl-2-(6-trifluoromethyl-benzothiazol-2-ylamino)-1H- |
| | benzoimidazole-5-carboxylic acid ((S)-5-amino-1-dimethylcarbamoyl- |
| | pentyl)-amide hydrochloride |
| 370 | 1-Methyl-2-(6-trifluoromethyl-benzothiazol-2-ylamino)-1H- |
| | benzoimidazole-5-carboxylic acid ((S)-5-dimethylamino-1- |
| | dimethylcarbamoyl-pentyl)-amide |
| 371 | 2-(6-Chloro-benzothiazol-2-ylamino)-1-methyl-1H-benzoimidazole-5- |
| | carboxylic acid (2-dimethylcarbamoyl-ethyl)-amide |
| 372 | 1-Methyl-2-(6-trifluoromethyl-benzothiazol-2-ylamino)-1H- |
| | benzoimidazole-5-carboxylic acid (2-dimethylcarbamoyl-ethyl)-amide |
| 373 | 2-(6-Chloro-benzothiazol-2-ylamino)-1-methyl-1H-benzoimidazole-5- |
| | carboxylic acid (3-morpholin-4-yl-3-oxo-propyl)-amide |
| 374 | 1-Methyl-2-(6-trifluoromethyl-benzothiazol-2-ylamino)-1H- |
| | benzoimidazole-5-carboxylic acid (3-morpholin-4-yl-3-oxo-propyl)-amide |
| 375 | 1-Methyl-2-(6-trifluoromethyl-benzothiazol-2-ylamino)-1H- |
| | benzoimidazole-5-carboxylic acid [3-(4-methyl-piperazin-1-yl)-3-oxo- |
| | propyl]-amide |
| 376 | 2-(6-Chloro-benzothiazol-2-ylamino)-1-methyl-1H-benzoimidazole-5- |
| | carboxylic acid (3-dimethylcarbamoyl-propyl)-amide |
| 377 | 2-(6-Chloro-benzothiazol-2-ylamino)-1-methyl-1H-benzoimidazole-5- |
| | carboxylic acid [4-(4-methyl-piperazin-1-yl)-4-oxo-butyl]-amide |
| 378 | 4-{[2-(6-Chloro-benzothiazol-2-ylamino)-1-methyl-1H-benzoimidazole-5- |
| | carbonyl]-amino}-trans-cyclohexanecarboxylic acid |
| 379 | 2-(6-Chloro-benzothiazol-2-ylamino)-1-methyl-1H-benzoimidazole-5- |
| | carboxylic acid (4-trans-dimethylcarbamoyl-cyclohexyl)-amide |
| 380 | 2-(6-Chloro-benzothiazol-2-ylamino)-1-methyl-1H-benzoimidazole-5- |
| | carboxylic acid methylcarbamoylmethyl-amide |
| 381 | 1-Methyl-2-(6-trifluoromethyl-benzothiazol-2-ylamino)-1H- |
| | benzoimidazole-5-carboxylic acid methylcarbamoylmethyl-amide |
| 382 | 2-(6-Chloro-benzothiazol-2-ylamino)-1-methyl-1H-benzoimidazole-5- |
| | carboxylic acid [((R)-2-hydroxy-propylcarbamoyl)-methyl]-amide |
| 383 | 2-(6-Chloro-benzothiazol-2-ylamino)-1-methyl-1H-benzoimidazole-5- |
| | carboxylic acid [(2-methanesulfonyl-ethylcarbamoyl)-methyl]-amide |
| 384 | 2-(6-Chloro-benzothiazol-2-ylamino)-1-methyl-1H-benzoimidazole-5- |
| | carboxylic acid [(tetrahydro-furan-3-ylcarbamoyl)-methyl]-amide |
| 385 | 2-(6-Chloro-benzothiazol-2-ylamino)-1-methyl-1H-benzoimidazole-5- |
| | carboxylic acid [(tetrahydro-pyran-4-ylcarbamoyl)-methyl]-amide |
| 386 | 2-(6-Chloro-benzothiazol-2-ylamino)-1-methyl-1H-benzoimidazole-5- |
| | carboxylic acid [(1-methyl-piperidin-4-ylcarbamoyl)-methyl]-amide |
| 387 | 2-(6-Chloro-benzothiazol-2-ylamino)-1-methyl-1H-benzoimidazole-5- |
| | carboxylic acid ((R)-piperidin-3-ylcarbamoylmethyl)-amide hydrochloride |
| 388 | 2-(6-Chloro-benzothiazol-2-ylamino)-1-methyl-1H-benzoimidazole-5- |
| | carboxylic acid [((R)-1-methyl-piperidin-3-ylcarbamoyl)-methyl]-amide |
| 389 | 1-Methyl-2-(6-trifluoromethoxy-benzothiazol-2-ylamino)-1H- |
| | benzoimidazole-5-carboxylic acid [(4-hydroxy-benzylcarbamoyl)-methyl]-amide |
| 390 | 1-Methyl-2-(6-trifluoromethoxy-benzothiazol-2-ylamino)-1H- |
| | benzoimidazole-5-carboxylic acid {[(2-hydroxy-ethyl)-methyl-carbamoyl]- |
| | methyl}-amide |
| 391 | 2-(6-Chloro-benzothiazol-2-ylamino)-1-methyl-1H-benzoimidazole-5- |
| | carboxylic acid {[(2-hydroxy-ethyl)-methyl-carbamoyl]-methyl}-amide |
| 392 | 1-Methyl-2-(6-trifluoromethoxy-benzothiazol-2-ylamino)-1H- |
| | benzoimidazole-5-carboxylic acid {[bis-(2-hydroxy-ethyl)-carbamoyl]- |
| | methyl}-amide |
| 393 | 2-(6-Chloro-benzothiazol-2-ylamino)-1-methyl-1H-benzoimidazole-5- |
| | carboxylic acid {[methyl-(tetrahydro-pyran-4-yl)-carbamoyl]-methyl}-amide |
| 394 | 2-(6-Chloro-benzothiazol-2-ylamino)-1-methyl-1H-benzoimidazole-5- |
| | carboxylic acid [(methyl-pyrrolidin-3-yl-carbamoyl)-methyl]-amide |
| | hydrochloride |
| 395 | 2-(6-Chloro-benzothiazol-2-ylamino)-1-methyl-1H-benzoimidazole-5- |
| | carboxylic acid {[methyl-(1-methyl-pyrrolidin-3-yl)-carbamoyl]-methyl}-amide |
| 396 | 2-(6-Chloro-benzothiazol-2-ylamino)-1-methyl-1H-benzoimidazole-5- |
| | carboxylic acid [(methyl-piperidin-3-yl-carbamoyl)-methyl]-amide |
| | hydrochloride |
| 397 | 2-(6-Chloro-benzothiazol-2-ylamino)-1-methyl-1H-benzoimidazole-5- |
| | carboxylic acid (2-oxo-2-pyrrolidin-1-yl-ethyl)-amide |
| 398 | 1-Methyl-2-(6-trifluoromethoxy-benzothiazol-2-ylamino)-1H- |
| | benzoimidazole-5-carboxylic acid [2-(3-hydroxy-pyrrolidin-1-yl)-2-oxo- |
| | ethyl]-amide |
| 399 | 2-(6-Chloro-benzothiazol-2-ylamino)-1-methyl-1H-benzoimidazole-5- |
| | carboxylic acid [2-(3-hydroxy-pyrrolidin-1-yl)-2-oxo-ethyl]-amide |
| 400 | 2-(6-Chloro-benzothiazol-2-ylamino)-1-methyl-1H-benzoimidazole-5- |
| | carboxylic acid [2-((R)-3-hydroxy-pyrrolidin-1-yl)-2-oxo-ethyl]-amide |
| 401 | 2-(6-Chloro-benzothiazol-2-ylamino)-1-methyl-1H-benzoimidazole-5- |
| | carboxylic acid [2-((S)-3-hydroxy-pyrrolidin-1-yl)-2-oxo-ethyl]-amide |
| 402 | 1-Methyl-2-(6-trifluoromethyl-benzothiazol-2-ylamino)-1H- |
| | benzoimidazole-5-carboxylic acid [2-((R)-3-hydroxy-pyrrolidin-1-yl)-2-oxo- |
| | ethyl]-amide |
| 403 | 1-Methyl-2-(6-trifluoromethyl-benzothiazol-2-ylamino)-1H- |
| | benzoimidazole-5-carboxylic acid [2-((S)-3-hydroxy-pyrrolidin-1-yl)-2-oxo- |
| | ethyl]-amide |
| 404 | 2-(6-Chloro-benzothiazol-2-ylamino)-1-(2-methoxy-ethyl)-1H- |
| | benzoimidazole-5-carboxylic acid [2-((R)-3-hydroxy-pyrrolidin-1-yl)-2-oxo- |
| | ethyl]-amide |
| 405 | 2-(6-Chloro-benzothiazol-2-ylamino)-1-(2-methoxy-ethyl)-1H- |
| | benzoimidazole-5-carboxylic acid [2-((S)-3-hydroxy-pyrrolidin-1-yl)-2-oxo- |
| | ethyl]-amide |
| 406 | 1-Methyl-2-(6-trifluoromethoxy-benzothiazol-2-ylamino)-1H- |
| | benzoimidazole-5-carboxylic acid [2-((S)-2-hydroxymethyl-pyrrolidin-1-yl)- |
| | 2-oxo-ethyl]-amide |
| 407 | 1-Methyl-2-(6-trifluoromethoxy-benzothiazol-2-ylamino)-1H- |
| | benzoimidazole-5-carboxylic acid [2-((3S,4S)-3,4-dihydroxy-pyrrolidin-1- |
| | yl)-2-oxo-ethyl]-amide |
| 408 | 2-(6-Chloro-benzothiazol-2-ylamino)-1-methyl-1H-benzoimidazole-5- |
| | carboxylic acid [2-((R)-3-methoxy-pyrrolidin-1-yl)-2-oxo-ethyl]-amide |
| 409 | 2-(6-Chloro-benzothiazol-2-ylamino)-1-methyl-1H-benzoimidazole-5- |
| | carboxylic acid [2-((S)-3-methoxy-pyrrolidin-1-yl)-2-oxo-ethyl]-amide |
| 410 | 1-Methyl-2-(6-trifluoromethyl-benzothiazol-2-ylamino)-1H- |
| | benzoimidazole-5-carboxylic acid [2-((S)-3-methoxy-pyrrolidin-1-yl)-2-oxo- |
| | ethyl]-amide |
| 411 | 2-(6-Chloro-benzothiazol-2-ylamino)-1-methyl-1H-benzoimidazole-5- |
| | carboxylic acid [2-((S)-3-amino-pyrrolidin-1-yl)-2-oxo-ethyl]-amide |
| | hydrochloride |
| 412 | 1-Methyl-2-(6-trifluoromethoxy-benzothiazol-2-ylamino)-1H- |
| | benzoimidazole-5-carboxylic acid [2-((S)-3-amino-pyrrolidin-1-yl)-2-oxo- |
| | ethyl]-amide hydrochloride |
| 413 | 1-Methyl-2-(6-trifluoromethoxy-benzothiazol-2-ylamino)-1H- |
| | benzoimidazole-5-carboxylic acid [2-((R)-3-methylamino-pyrrolidin-1-yl)-2- |
| | oxo-ethyl]-amide hydrochloride |
| 414 | 1-Methyl-2-(6-trifluoromethoxy-benzothiazol-2-ylamino)-1H- |
| | benzoimidazole-5-carboxylic acid [2-((S)-3-methylamino-pyrrolidin-1-yl)-2- |
| | oxo-ethyl]-amide hydrochloride |
| 415 | 1-Methyl-2-(6-trifluoromethoxy-benzothiazol-2-ylamino)-1H- |
| | benzoimidazole-5-carboxylic acid [2-((R)-3-dimethylamino-pyrrolidin-1-yl)- |
| | 2-oxo-ethyl]-amide |
| 416 | 1-Methyl-2-(6-trifluoromethoxy-benzothiazol-2-ylamino)-1H- |
| | benzoimidazole-5-carboxylic acid [2-((S)-3-dimethylamino-pyrrolidin-1-yl)- |
| | 2-oxo-ethyl]-amide |
| 417 | 2-(6-Chloro-benzothiazol-2-ylamino)-1-methyl-1H-benzoimidazole-5- |
| | carboxylic acid [2-((R)-3-dimethylamino-pyrrolidin-1-yl)-2-oxo-ethyl]-amide |
| 418 | 2-(6-Chloro-benzothiazol-2-ylamino)-1-methyl-1H-benzoimidazole-5- |
| | carboxylic acid [2-((S)-3-dimethylamino-pyrrolidin-1-yl)-2-oxo-ethyl]-amide |
| 419 | 2-(6-Chloro-benzothiazol-2-ylamino)-1-methyl-1H-benzoimidazole-5- |
| | carboxylic acid [2-((S)-2-dimethylcarbamoyl-pyrrolidin-1-yl)-2-oxo-ethyl]-amide |
| 420 | 3-Methyl-2-(6-trifluoromethoxy-benzothiazol-2-ylamino)-3H-imidazo [4,5- |
| | b]pyridine-6-carboxylic acid (2-morpholin-4-yl-2-oxo-ethyl)-amide |
| 421 | 2-(6-Chloro-benzothiazol-2-ylamino)-1-methyl-1H-benzoimidazole-5- |
| | carboxylic acid (2-morpholin-4-yl-2-oxo-ethyl)-amide |
| 422 | 1-Methyl-2-(6-trifluoromethyl-benzothiazol-2-ylamino)-1H- |
| | benzoimidazole-5-carboxylic acid (2-morpholin-4-yl-2-oxo-ethyl)-amide |
| 423 | 2-(6-Chloro-benzothiazol-2-ylamino)-1-methyl-1H-benzoimidazole-5- |
| | carboxylic acid (2-oxo-2-thiomorpholin-4-yl-ethyl)-amide |
| 424 | 2-(6-Chloro-benzothiazol-2-ylamino)-1-methyl-1H-benzoimidazole-5- |
| | carboxylic acid [2-(1,1-dioxo-thiomorpholin-4-yl)-2-oxo-ethyl]-amide |
| 425 | 2-(6-Chloro-benzothiazol-2-ylamino)-1-methyl-1H-benzoimidazole-5- |
| | carboxylic acid (2-oxo-2-piperazin-1-yl-ethyl)-amide hydrochloride |
| 426 | 2-(6-Chloro-benzothiazol-2-ylamino)-1-methyl-1H-benzoimidazole-5- |
| | carboxylic acid [2-(4-methyl-piperazin-1-yl)-2-oxo-ethyl]-amide |
| | 1-Methyl-2-(6-trifluoromethyl-benzothiazol-2-ylamino)-1H- |
| 427 | benzoimidazole-5-carboxylic acid [2-(4-methyl-piperazin-1-yl)-2-oxo-ethyl]-amide |
| 428 | 2-(6-Chloro-benzothiazol-2-ylamino)-1-(2-methoxy-ethyl)-1H- |
| | benzoimidazole-5-carboxylic acid [2-(4-methyl-piperazin-1-yl)-2-oxo-ethyl]-amide |
| 429 | 2-(6-Chloro-benzothiazol-2-ylamino)-1-methyl-1H-benzoimidazole-5- |
| | carboxylic acid [2-(4-methanesulfonyl-piperazin-1-yl)-2-oxo-ethyl]-amide |
| 430 | 2-(6-Chloro-benzothiazol-2-ylamino)-1-methyl-1H-benzoimidazole-5- |
| | carboxylic acid [2-(4-dimethylsulfamoyl-piperazin-1-yl)-2-oxo-ethyl]-amide |
| 431 | 1-Methyl-2-(6-trifluoromethoxy-benzothiazol-2-ylamino)-1H- |
| | benzoimidazole-5-carboxylic acid [2-(3-hydroxy-piperidin-1-yl)-2-oxo- |
| | ethyl]-amide |
| 432 | 1-Methyl-2-(6-trifluoromethyl-benzothiazol-2-ylamino)-1H- |
| | benzoimidazole-5-carboxylic acid [2-((R)-3-hydroxy-piperidin-1-yl)-2-oxo- |
| | ethyl]-amide |
| 433 | 2-(6-Chloro-benzothiazol-2-ylamino)-1-methyl-1H-benzoimidazole-5- |
| | carboxylic acid [2-((S)-3-hydroxy-piperidin-1-yl)-2-oxo-ethyl]-amide |
| 434 | 1-Methyl-2-(6-trifluoromethyl-benzothiazol-2-ylamino)-1H- |
| | benzoimidazole-5-carboxylic acid [2-((S)-3-hydroxy-piperidin-1-yl)-2-oxo- |
| | ethyl]-amide |
| 435 | 1-Methyl-2-(6-trifluoromethoxy-benzothiazol-2-ylamino)-1H- |
| | benzoimidazole-5-carboxylic acid [2-(4-hydroxy-piperidin-1-yl)-2-oxo- |
| | ethyl]-amide |
| 436 | 2-(6-Chloro-benzothiazol-2-ylamino)-1-methyl-1H-benzoimidazole-5- |
| | carboxylic acid [2-(4-hydroxy-piperidin-1-yl)-2-oxo-ethyl]-amide |
| 437 | 1-Methyl-2-(6-trifluoromethyl-benzothiazol-2-ylamino)-1H- |
| | benzoimidazole-5-carboxylic acid [2-(4-hydroxy-piperidin-1-yl)-2-oxo- |
| | ethyl]-amide |
| 438 | 2-(6-Chloro-benzothiazol-2-ylamino)-1-(2-methoxy-ethyl)-1H- |
| | benzoimidazole-5-carboxylic acid [2-(4-hydroxy-piperidin-1-yl)-2-oxo- |
| | ethyl]-amide |
| 439 | 1-Methyl-2-(6-trifluoromethoxy-benzothiazol-2-ylamino)-1H- |
| | benzoimidazole-5-carboxylic acid [2-(2-hydroxymethyl-piperidin-1-yl)-2- |
| | oxo-ethyl]-amide |
| 440 | 1-Methyl-2-(6-trifluoromethoxy-benzothiazol-2-ylamino)-1H- |
| | benzoimidazole-5-carboxylic acid [2-(3-hydroxymethyl-piperidin-1-yl)-2- |
| | oxo-ethyl]-amide |
| 441 | 1-Methyl-2-(6-trifluoromethoxy-benzothiazol-2-ylamino)-1H- |
| | benzoimidazole-5-carboxylic acid [2-(4-hydroxymethyl-piperidin-1-yl)-2- |
| | oxo-ethyl]-amide |
| 442 | 2-(6-Chloro-benzothiazol-2-ylamino)-1-methyl-1H-benzoimidazole-5- |
| | carboxylic acid [2-(4-hydroxymethyl-piperidin-1-yl)-2-oxo-ethyl]-amide |
| 443 | 2-(6-Chloro-benzothiazol-2-ylamino)-1-(2-methoxy-ethyl)-1H- |
| | benzoimidazole-5-carboxylic acid [2-(4-hydroxymethyl-piperidin-1-yl)-2- |
| | oxo-ethyl]-amide |
| 444 | 2-(6-Chloro-benzothiazol-2-ylamino)-1-methyl-1H-benzoimidazole-5- |
| | carboxylic acid [2-((S)-3-methoxy-piperidin-1-yl)-2-oxo-ethyl]-amide |
| 445 | 1-Methyl-2-(6-trifluoromethyl-benzothiazol-2-ylamino)-1H- |
| | benzoimidazole-5-carboxylic acid [2-((S)-3-methoxy-piperidin-1-yl)-2-oxo- |
| | ethyl]-amide |
| 446 | 1-Methyl-2-(6-trifluoromethyl-benzothiazol-2-ylamino)-1H- |
| | benzoimidazole-5-carboxylic acid [2-((R)-3-methoxy-piperidin-1-yl)-2-oxo- |
| | ethyl]-amide |
| 447 | 1-Methyl-2-(6-trifluoromethoxy-benzothiazol-2-ylamino)-1H- |
| | benzoimidazole-5-carboxylic acid [2-(4-methoxymethyl-piperidin-1-yl)-2- |
| | oxo-ethyl]-amide |
| 448 | 1-Methyl-2-(6-trifluoromethoxy-benzothiazol-2-ylamino)-1H- |
| | benzoimidazole-5-carboxylic acid [2-(4-fluoromethyl-piperidin-1-yl)-2-oxo- |
| | ethyl]-amide |
| 449 | 1-Methyl-2-(6-trifluoromethoxy-benzothiazol-2-ylamino)-1H- |
| | benzoimidazole-5-carboxylic acid [2-oxo-2-(4-trifluoromethyl-piperidin-1- |
| | yl)-ethyl]-amide |
| 450 | 1-Methyl-2-(6-trifluoromethoxy-benzothiazol-2-ylamino)-1H- |
| | benzoimidazole-5-carboxylic acid [2-(4-cyano-piperidin-1-yl)-2-oxo-ethyl]-amide |
| 451 | 1-Methyl-2-(6-trifluoromethoxy-benzothiazol-2-ylamino)-1H- |
| | benzoimidazole-5-carboxylic acid [2-(4-carbamoyl-piperidin-1-yl)-2-oxo- |
| | ethyl]-amide |
| 452 | 1-Methyl-2-(6-trifluoromethoxy-benzothiazol-2-ylamino)-1H- |
| | benzoimidazole-5-carboxylic acid {2-oxo-2-[4-(pyrimidin-2-yloxy)- |
| | piperidin-1-yl]-ethyl}-amide |
| 453 | 1-Methyl-2-(6-trifluoromethoxy-benzothiazol-2-ylamino)-1H- |
| | benzoimidazole-5-carboxylic acid [2-(4-methylamino-piperidin-1-yl)-2-oxo- |
| | ethyl]-amide hydrochloride |
| 454 | 2-(6-Chloro-benzothiazol-2-ylamino-1-methyl-1H-benzoimidazole-5- |
| | carboxylic acid [2-(4-methylamino-piperidin-1-yl)-2-oxo-ethyl]-amide |
| | hydrochloride |
| 455 | 1-Methyl-2-(6-trifluoromethoxy-benzothiazol-2-ylamino)-1H- |
| | benzoimidazole-5-carboxylic acid [2-(4-dimethylamino-piperidin-1-yl)-2- |
| | oxo-ethyl]-amide |
| 456 | 2-(6-Chloro-benzothiazol-2-ylamino)-1-methyl-1H-benzoimidazole-5- |
| | carboxylic acid [2-(4-dimethylamino-piperidin-1-yl)-2-oxo-ethyl]-amide |
| 457 | 2-(6-Chloro-benzothiazol-2-ylamino)-1-methyl-1H-benzoimidazole-5- |
| | carboxylic acid [2-(4-methylaminomethyl-piperidin-1-yl)-2-oxo-ethyl]- |
| | amide hydrochloride |
| 458 | 1-Methyl-2-(6-trifluoromethoxy-benzothiazol-2-ylamino)-1H- |
| | benzoimidazole-5-carboxylic acid [2-(4-dimethylaminomethyl-piperidin-1- |
| | yl)-2-oxo-ethyl]-amide |
| 459 | 2-(6-Chloro-benzothiazol-2-ylamino)-1-methyl-1H-benzoimidazole-5- |
| | carboxylic acid [2-(4-dimethylaminomethyl-piperidin-1-yl)-2-oxo-ethyl]-amide |
| 460 | 2-(6-Chloro-benzothiazol-2-ylamino)-1-methyl-1H-benzoimidazole-5- |
| | carboxylic acid [2-(3-methylaminomethyl-piperidin-1-yl)-2-oxo-ethyl]-amid |
| | hydrochloride |
| 461 | 2-(6-Chloro-benzothiazol-2-ylamino)-1-methyl-1H-benzoimidazole-5- |
| | carboxylic acid [2-(3-dimethylaminomethyl-piperidin-1-yl)-2-oxo-ethyl]-amide |
| 462 | 2-(6-Chloro-benzothiazol-2-ylamino)-1-methyl-1H-benzoimidazole-5- |
| | carboxylic acid ((S)-1-dimethylcarbamoyl-ethyl)-amide |
| 463 | 1-Methyl-2-(6-trifluoromethyl-benzothiazol-2-ylamino)-1H- |
| | benzoimidazole-5-carboxylic acid ((S)-1-dimethylcarbamoyl-ethyl)-amide |
| 464 | 2-(6-Chloro-benzothiazol-2-ylamino)-1-methyl-1H-benzoimidazole-5- |
| | carboxylic acid [(S)-1-methyl-2-(4-methyl-piperazin-1-yl)-2-oxo-ethyl]-amide |
| 465 | 2-(6-Chloro-benzothiazol-2-ylamino)-1-methyl-1H-benzoimidazole-5- |
| | carboxylic acid ((S)-1-methyl-2-morpholin-4-yl-2-oxo-ethyl)-amide |
| 466 | 1-Methyl-2-(6-trifluoromethyl-benzothiazol-2-ylamino)-1H- |
| | benzoimidazole-5-carboxylic acid ((S)-1-methyl-2-morpholin-4-yl-2-oxo- |
| | ethyl)-amide |
| 467 | 2-(6-Chloro-benzothiazol-2-ylamino)-1-methyl-1H-benzoimidazole-5- |
| | carboxylic acid ((R)-1-dimethylcarbamoyl-ethyl)-amide |
| 468 | 1-Methyl-2-(6-trifluoromethyl-benzothiazol-2-ylamino)-1H- |
| | benzoimidazole-5-carboxylic acid ((R)-1-dimethylcarbamoyl-ethyl)-amide |
| 469 | 2-(6-Chloro-benzothiazol-2-ylamino)-1-methyl-1H-benzoimidazole-5- |
| | carboxylic acid [(R)-1-methyl-2-(4-methyl-piperazin-1-yl)-2-oxo-ethyl]-amide |
| 470 | 1-Methyl-2-(6-trifluoromethyl-benzothiazol-2-ylamino)-1H- |
| | benzoimidazole-5-carboxylic acid [(R)-1-methyl-2-(4-methyl-piperazin-1- |
| | yl)-2-oxo-ethyl]-amide |
| 471 | 2-(6-Chloro-benzothiazol-2-ylamino)-1-methyl-1H-benzoimidazole-5- |
| | carboxylic acid ((R)-1-methyl-2-morpholin-4-yl-2-oxo-ethyl)-amide |
| 472 | 1-Methyl-2-(6-trifluoromethyl-benzothiazol-2-ylamino)-1H- |
| | benzoimidazole-5-carboxylic acid ((R)-1-methyl-2-morpholin-4-yl-2-oxo- |
| | ethyl)-amide |
| 473 | N-[2-(2-Hydroxyethoxy)ethyl]-3-methyl-2-[[6-(trifluoromethyl)-1,3- |
| | benzothiazol-2-yl]amino]imidazo[4,5-b]pyridine-6-carboxamide |
| 474 | 1-Ethyl-N-[2-(2-hydroxyethoxy)ethyl]-2-[[5-(trifluoromethoxy)-1,3- |
| | benzothiazol-2-yl]amino]benzimidazole-5-carboxamide |

Compounds 1-474 in Table A may be prepared as described in WO '018 or other methods apparent to one of skill in the art. For example, Compounds 473 and 474 in Table A may be prepared as described in the Examples section below.

In another aspect, the present invention provides a pharmaceutical composition comprising the compound of Formula (I) or a pharmaceutically acceptable salt thereof for use in treating sickle cell disease or related disorders. In an embodiment, the present invention provides a pharmaceutical composition comprising a compound (or salt) of any one of embodiments 1 to 250 (recited above) and a pharmaceutical carrier. In another embodiment, the pharmaceutical composition comprises a compound (or salt) of any one of the examples and a pharmaceutically acceptable carrier.

Thus, in another embodiment, the invention provides a pharmaceutical composition comprising a compound of Formula (I) or a pharmaceutically acceptable salt thereof, and a pharmaceutically acceptable carrier. In another embodiment, the invention provides a pharmaceutical composition comprising a compound (or salt) of any one of embodiments 1 to 250 and a pharmaceutical acceptable carrier.

In another embodiment, the present invention provides a compound of Formula (I) or a pharmaceutically acceptable salt thereof for use in medicine. In another embodiment, the invention provides a compound (or salt) of any one of embodiments 1 to 250 for use in medicine.

### B. Co-Administration

The present invention further provides for the use of a compound of Formula (I), or a pharmaceutically acceptable salt thereof, in combination with one or more active compounds for simultaneous, subsequent, or sequential administration. The invention also provides for the use of a compound (or salt) of any one of embodiments 1 to 250 in combination with one or more medically effective active compounds for simultaneous, subsequent, or sequential administration. Examples of such active ingredients include, but are not limited to, HU, Nrf2 activators, antioxidants, detoxification agents, and anti-inflammatory agents. In one embodiment, the invention provides a pharmaceutical composition comprising a compound (or salt) of any one of embodiments 1 to 250 and at least one other medically effective active ingredient selected from HU, Nrf2 activators, antioxidants, detoxification agents, and anti-inflammatory agents. In another embodiment, the invention provides for the use of a compound (or salt) of any one of embodiments 1 to 250 in combination with at least one other medically effective active ingredient selected from Nrf2 activators, antioxidants, detoxification agents, and anti-inflammatory agents for simultaneous, subsequent, or sequential administration.

Nrf2 Activators may comprise a Michael addition acceptor, one or more fumaric acid esters, i.e. fumaric acid mono- and/or diesters which may be selected from the group of monoalkyl hydrogen fumarate and dialkyl fumarate, such as monomethyl hydrogen fumarate, dimethyl fumarate, monoethyl hydrogen fumarate, and diethyl fumarate, furthermore ethacrynic acid, bardoxolone methyl (methyl 2-cyano-3,12-dioxooleana-1,9(11) dien-28-oate), isothiocyanate such as sulforaphane, 1,2-dithiole-3-thione such as oltipraz, 3,5-di-tert-butyl-4-hydroxytoluene, 3-hydroxycoumarin, or a pharmacologically active derivative or analog of the aforementioned agents. In an embodiment, Nrf2 Activators for use in combination with a compound of the invention are bardoxolone methyl and fumaric acid esters.

Nrf2 Activators compounds may be classified based on their chemical structures: Diphenols, Michael reaction acceptors, isothiocyanates, thiocarbamates, trivalent arsenicals, 1,2-dithiole-3-thiones, hydroperoxides, vicinal dimercaptans, heavy metals, and polyenes. In general, Nrf2 Activators are chemically reactive in that they may be electrophiles, substrates for glutathione transferases, and/or can modify sulfhydryl groups by alkylation, oxidation, or reduction.

In another embodiment, the Nrf2 activators are bardoxolone methyl and dialkyl fumarate such as dimethyl fumarate and diethyl fumarate.

In another embodiment, Nrf2 activators are selected from: Chalcone derivatives such as 2-trifluoromethyl-2′-methoxychalcone, auranofin, ebselen, 1,2-naphthoquinone, cynnamic aldehyde, caffeic acid and its esters, curcumin, reservatrol, artesunate, tert-butylhydroquinone, and -quinone, (tBHQ, tBQ), vitamins K1, K2 and K3, menadione, fumaric acid esters, i.e. fumaric acid mono- and/or diester which may be selected from the group of monoalkyl hydrogen fumarate and dialkyl fumarate, such as monomethyl hydrogen fumarate, dimethyl fumarate (DMF), monoethyl hydrogen fumarate, and diethyl fumarate, 2-cyclopentenones, ethacrynic acid and its alkyl esters, bardoxolone methyl (methyl 2-cyano-3, 12-dioxooleana-1,9(11) dien-28-oate) (CDDO-Me, RTA 402), ethyl 2-cyano-3,12-dioxooleana-1,9(11) dien-28-oate, 2-cyano-3,12-dioxooleana-1,9(11) dien-28-oic acid (CDDO), 1 [2-Cyano-3,12-dioxooleana-1,9(11)-dien-28-oyl] imidazole (CDDO-Im), (2-cyano-N-methyl-3,12-dioxooleana-1,9(11)-dien-28 amide (CDDO-methyl amide, CDDO-MA), isothiocyanate such as sulforaphane, 1,2-dithiole-3-thione such as oltipraz, 3,5-di-tert-butyl-4-hydroxytoluene, 3-hydroxycoumarin, 4-hydroxynonenal, 4-oxononenal, malondialdehyde, (E)-2-hexenal, capsaicin, allicin, allylisothiocyanate, 6-methylthiohexyl isothiocyanate, 7-methylthioheptyl isothiocyanate, sulforaphane, 8-methylthiooctyl isothiocyanate, corticosteroids, such as dexamethasone, 8-iso prostaglandin A2, alkyl pyruvate, such as methyl and ethyl pyruvate, diethyl or dimethyl oxaloproprionate, 2-acetamidoacrylate, methyl or ethyl-2-acetamidoacrylate, hypoestoxide, parthenolide, eriodictyol, 4-hydroxy-2-nonenal, 4-oxo-2nonenal, geranial, zerumbone, aurone, isoliquiritigenin, xanthohumol, [10]-Shogaol, eugenol, 1′-acetoxychavicol acetate, allyl isothiocyanate, benzyl isothiocyanate, phenethyl isothiocyanate, 4-(methylthio)-3-butenyl isothiocyanate and 6-methylsulfinylhexyl isothiocyanate, ferulic acid and its esters, such as ferulic acid ethyl ester, and ferulic acid methyl ester, sofalcone, 4-methyl daphnetin, imperatorin, auraptene, poncimarin, bis [2-hydroxybenzylidene] acetones, alicylcurcuminoid, 4-bromo flavone, beta-naphthoflavone, sappanone A, aurones and its corresponding indole derivatives such as benzylidene-indolin-2-ones, perillaldehyde, quercetin, fisetin, koparin, genistein, tanshinone HA, BHA, BHT, PMX-290, AL-1, avicin D, gedunin, fisetin, andrographolide, and tricyclic bis (cyano enone) TBE-31 [(+/−)-(4bS,8aR,10aS)-10a-ethynyl-4-b,8,8-trimethyl-3,7-dioxo-3,4-b,7,8, -8a,9,10,10a-octahydrophenanthrene-2,6-dicarbonitrile].

In another embodiment, Nrf2 activators are selected from: carnosic acid, 2-naphthoquinone, cynnamic aldehyde, caffeic acid and its esters, curcumin, reservatrol, artesunate, tert-butylhydroquinone, vitamins K1, K2 and K3, fumaric acid esters, i.e. fumaric acid mono- and/or diester which is preferably selected from the group of monoalkyl hydrogen fumarate and dialkyl fumarate, such as monomethyl hydrogen fumarate, dimethyl fumarate, monoethyl hydrogen fumarate, and diethyl fumarate, isothiocyanate such as sulforaphane, 1,2-dithiole-3-thione such as oltipraz, 3,5-di-tert-butyl-4-hydroxytoluene, 3-hydroxycoumarin, 4-hydroxynonenal, 4-oxononenal, malondialdehyde, (E)-2-hexenal, capsaicin, allicin, allylisothiocyanate, 6-methylthiohexyl isothiocyanate, 7-methylthioheptyl isothiocyanate, sulforaphane, 8-methylthiooctyl isothiocyanate, 8-iso prostaglandin A2, alkyl pyruvate, such as methyl and ethyl pyruvate, diethyl or dimethyl oxaloproprionate, 2-acetamidoacrylate, methyl or ethyl-2-acetamidoacrylate, hypoestoxide, parthenolide, eriodictyol, 4-Hydroxy-2-nonenal, 4-oxo-2nonenal, geranial, zerumbone, aurone, isoliquiritigenin, xanthohumol, [10]-Shogaol, eugenol, 1′-acetoxychavicol acetate, allyl isothiocyanate, benzyl isothiocyanate, phenethyl isothiocyanate, 4-(Methylthio)-3-butenyl isothiocyanate and 6-methylsulfinylhexyl isothiocyanate and the respective quinone or hydroquinone forms of the aforementioned quinone and hydroquinone derivatives.

In another embodiment, Nrf2 Activators may be Michael reaction acceptors such as dimethylfumarate, monomethyl hydrogen fumarate isothiocyanates and 1,2-dithiole-3-thiones. In another embodiment, Nrf2 Activators are selected from monomethyl hydrogen fumarate, dimethyl fumarate, oltipraz, 1,2-naphthoquinone, tert-butylhydroquinone, methyl or ethyl pyruvate, 3,5-di-tert-butyl-4-hydroxytoluene, diethyl and dimethyl oxaloproprionate, hypoestoxide, parthenolide, eriodictyol, 4-Hydroxy-2-nonenal, 4-oxo-2nonenal, geranial, zerumbone, aurone, isoliquiritigenin, xanthohumol, [10]-Shogaol, eugenol, 1′-acetoxychavicol acetate, allyl isothiocyanate, benzyl isothiocyanate, phenethyl isothiocyanate, 4-(Methylthio)-3-butenyl isothiocyanate and 6-Methylsulfinylhexyl isothiocyanate.

Examples of the antioxidants include vitamin C, vitamin E, carotenoids, retinolds, polyphenols, flavonoids, lignan, selenium, butylated hydroxyanisole, ethylene diamine tetra-acetate, calcium disodium, acetylcysteine, probucol, and tempo.

Examples of the detoxification agents include dimethyl caprol, glutathione, acetylcysteine, methionine, sodium hydrogen carbonate, deferoxamine mesylate, calcium disodium edetate, trientine hydrochloride, penicillamine, and pharmaceutical charcoal.

The anti-inflammatory agents include steroidal anti-inflammatory agents and non-steroidal anti-inflammatory agents. Examples of the steroidal anti-inflammatory agents include cortisone acetate, hydrocortisone, paramethasone acetate, prednisolone, prednisolone, methylprednine, dexamethasone, triamcinolone, and betamethasone. Examples of the non-steroidal anti-inflammatory agents include salicylic acid non-steroidal anti-inflammatory agents such as aspirin, difiunisal, aspirin+ascorbic acid, and aspirin dialuminate; aryl acid non-steroidal anti-inflammatory agents such as diclofenac sodium, sulindac, fenbufen, indomethacin, indomethacin farnesyl, acemetacin, proglumetacin maleate, anfenac sodium, nabmeton, mofezolac, and etodorag; fenamic acid non-steroidal anti-inflammatory agents such as mefenamic acid, flufenamic acid aluminum, tolfenamic acid, and floctafenine; propionic acid non-steroidal anti-inflammatory agents such as ibuprofen, flurbiprofen, ketoprofen, naproxen, pranoprofen, fenoprofen calcium, thiaprofen, oxaprozin, loxoprofen sodium, alminoprofen, and zaltoprofen; oxicam non-steroldal anti-inflammatory agents such as piroxicam, ampiroxicam, tenoxicam, lornoxicam, and meloxicam; and basic non-steroidal anti-inflammatory agents such as tiaramide hydrochloride, epirizole, and emorfazone.

An appropriate time course for sequential administration may be chosen by the physician, according to such factors as the nature of a patient's illness, and the patient's condition for administration of individual active agents. In certain embodiments, sequential administration includes the co-administration of one or more additional active agents within a period of one week, 72 hours, 48 hours, 24 hours, or 12 hours.

In some embodiments, the compositions disclosed herein are co-administered in combination with one or more additional active agents for treatment of sickle cell disease, beta-thalassemia, or a related disorder. Such additional active agents may include, but are not limited to, folic acid, penicillin or another antibiotics, preferably a quinolone or macrolide, antivirals, anti-malarial prophylactics, and analgesics to control pain crises.

In some embodiments, the compositions are co-administered with one or more additional agents that increase expression of HbF, for example, hydroxyurea (HU).

In some embodiments, the compositions are co-administered with one or more additional treatment protocols, for example, transfusion therapy, stem cell therapy, gene therapy, bone marrow transplants, dialysis or kidney transplant for kidney disease, gallbladder removal in people with gallstone disease, hip replacement for avascular necrosis of the hip, surgery for eye problems, and wound care for leg ulcers.

### C. Effective Amounts

In some embodiments, the compositions are administered in an amount effective to induce a pharmacological, physiological, or molecular effect compared to a control that is not administered the composition. In some embodiments, the compositions are administered to a subject in need thereof to increase expression of HbF in the subject.

Suitable controls are known in the art and can be determined based on the disease to be treated. Suitable controls include, but are not limited to a subject, or subjects without sickle cell disease, a beta-thalassemia, or a sickle cell related disorder; or a condition or status of a subject with the disease or disorder prior to initiation of the treatment.

### D. Dosages and Dosage Regimes

The selected dosage depends upon the desired therapeutic effect, on the route of administration, and on the duration of the treatment desired. Generally, dosage levels of 0.001 to 100 mg/kg of body weight daily are administered to mammals. Generally, for intravenous injection or infusion, dosage may be lower.

An appropriate dose of a compound of Formula (I) or a pharmaceutically acceptable salt thereof for use in the present invention, may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

A compound of Formula (I) or a pharmaceutically acceptable salt thereof may be administered in a daily dosage of between 0.1 mg and 15 mg per kg. In another embodiment, where the subject is a human, the daily dose may be between 1 mg and 1000 mg. In another embodiment, a compound of Formula (I) or a pharmaceutically acceptable salt thereof is administered in an amount from 10 mg/day to 1000 mg/day, or from 25 mg/day to 800 mg/day, or from 37 mg/day to 750 mg/day, or from 75 mg/day to 700 mg/day, or from 100 mg/day to 600 mg/day, or from 150 mg/day to 500 mg/day, or from 200 mg/day to 400 mg/day. In other embodiments, the previous daily periods of administration of an amount of a compound of Formula (I) or a pharmaceutically acceptable salt thereof may be changed to a period of every 6 hours, 12 hours, 48 hours, 72 hours, 96 hours, 1 week, or 2 weeks.

In some embodiments, the compositions comprising a fumaric acid ester, such as DMF, MMF, or a combination thereof, daily dosages for fumaric acid esters in a human can range from about 1 mg to about 5,000 mg, from about 10 mg to about 2,500 grams, or from about 50 mg to about 2,000 grams of a fumaric acid ester, or a pharmacologically active salt thereof. In another embodiment, an effective dose of DMF or MMF to be administered to a subject, for example orally, can be from about 0.1 g to about 1 g or more than 1 g per day; from about 200 mg to about 800 mg per day; from about 240 mg to about 720 mg per day; from about 480 mg to about 720 mg per day; or about 720 mg per day. The daily dose can be administered in separate administrations of 2, 3, 4, or 6 equal doses. In some embodiments of the one or more fumaric acid esters, or pharmacologically active salts, derivatives, analogues or prodrugs thereof are present in a pharmaceutical preparation. In some embodiments the composition is administered to the patient three times per day (TID). In some embodiments the pharmaceutical preparation is administered to the patient two times per day (BID). In some embodiments, the composition is administered at least one hour before or after food is consumed by the patient.

In some embodiments, the composition is administered as part of a dosing regimen. For example, the patient can be administered a first dose of the composition for a first dosing period; and a second dose of the composition for a second dosing period, optionally followed by one or more additional doses for one or more additional dosing periods. The first dosing period can be less than one week, one week, or more than one week.

In some embodiments the dosage regime is a dose escalating dosage regime. The first dose can be a low dose, followed by measurement of levels of HbF expression, and then the step of decreasing, maintaining, or increasing the dose.

The current labeled dosing of hydroxyurea for sickle cell disease calls for the administration of an initial dose of 15 mg/kg/day in the form of a single dose, with monitoring of the patient's blood count every 2 weeks. If the blood counts are in an acceptable range, the dose may be increased by 5 mg/kg/day every 12 weeks until the MTD of 35 mg/kg/day is reached. Pharmaceutical compositions can contain 1 mg/kg to 50 mg/kg of a fumaric acid ester, such as MMF, in combination with 1 mg/kg to 35 mg/kg of HU. The combination formulation can contain 5, 10, 15, 20, 25, 30, 35, 40, 45 or 50 mg/kg of HU.

### E. Formulations

Pharmaceutical compositions comprising a compound of the invention are disclosed. The pharmaceutical compositions may be for administration by oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in unit dosage forms appropriate for each route of administration.

Red blood cells, which are cells of erythroid lineage, are the primary producers of hemoglobin. Therefore, in an embodiment a compound of the invention or a pharmaceutical composition is administered to a subject in an effective amount to induce HbF in hematopoietic stems cells. Therefore, in some embodiments, a compound of the invention or a pharmaceutical composition is administered in an effective amount to induce HbF expression in cells of erythroid lineage in the bone marrow (i.e., the red bone marrow), the liver, the spleen, or combinations thereof.

In a further embodiment, a compound of the invention or a pharmaceutical composition induces HbF in cells synthesizing or committed to synthesize hemoglobin. For example, in preferred embodiments, a compound of the invention induces HbF in basophilic normoblast/early normoblast also commonly called erythroblast, polychromatophilic normoblast/intermediate normoblast, orthochromatic normoblast/late normoblast, or a combination thereof.

In some embodiments, a compound of the invention or a pharmaceutical composition is administered locally to the site in need of therapy. Although red blood cells are the primary producers of hemoglobin, other, non-hematopoietic cells, including macrophage, retinal pigment cells, and alveolar epithelial cells such as alveolar type II (ATII) cells and Clara cells may also synthesize hemoglobin. Therefore, in some embodiments, a compound of the invention or a pharmaceutical composition is administered locally to interfaces where oxygen-carbon dioxide diffusion occurs, including but not limited, to the eye or lungs.

In some embodiments, a compound of the invention or a pharmaceutical composition is administered locally to the eye to treat a retinopathy, or another ocular manifestation associated with sickle cell disease or a related disorder.

In an embodiment, the pharmaceutical compositions are formulated for oral delivery. Oral solid dosage forms are described generally in Remington's Pharmaceutical Sciences, 21st Ed. 2005 at Chapter 45. Solid dosage forms include tablets, capsules, pills, troches or lozenges, cachets, pellets, powders, or granules or incorporation of the material into particulate preparations of polymeric compounds such as polylactic acid, polyglycolic acid, etc., or into liposomes. Such compositions may influence the physical state, stability, rate of in vivo release, and rate of in vivo clearance of the disclosed. The compositions may be prepared in liquid form, or may be in dried powder (e.g., lyophilized) form. Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled C release oral formulations may be desirable. Compounds of the invention can be incorporated into an inert matrix which permits release by either diffusion or leaching mechanisms, e.g., gums. Slowly degenerating matrices may also be incorporated into the formulation.

For oral formulations, the location of release may be the stomach, the small intestine (the duodenum, the jejunem, or the ileum), or the large intestine.

### IV. Methods of Diagnosis

The methods of treatment disclosed herein can include a first step of selecting a subject for treatment. In some embodiments, the subject is selected for treatment when the subject exhibits one or more of the clinical symptoms of sickle cell disease, beta-thalassemia, or a related disorder such as those discussed above. In some embodiments, the subject is selected for treatment when the subject exhibits a genetic or biochemical indicator of sickle cell disease, beta-thalassemia, or a related disorder. For example, the subject can be selected for treatment based on identification of a genetic alteration, defect, or mutation in the beta-globin gene or an expression control sequence thereof, by biochemical or morphological alterations in hemoglobin or hemoglobin synthesizing cells, or combinations thereof.

In some embodiments, the subject is selected when a combination of clinical symptoms and genetic or biochemical alterations are identified. In some embodiments, the subject is selected based on one or more clinical symptoms, or one or more genetic or biochemical alterations. For example, subjects can be selected for treatment based on the identification of a genetic alteration, a biochemical or morphological alteration, or a combination thereof, before the subject exhibits clinical symptoms of sickle cell disease, beta-thalassemia, or a related disorder.

In some embodiments, the methods of treatment may further comprise the step of determining whether a subject is at risk for or has sickle cell disease, beta-thalassemia, or a related disorder by obtaining or having obtained a biological sample from the subject and performing or having performed a bodily fluid test on the biological sample to determine if the subject has one or more biomarkers or a genetic mutation associated with sickle cell disease, beta-thalassemia, or a related disorder. If the subject is determined to be at risk for or has sickle cell disease, beta-thalassemia, or a related disorder, the method further comprises administering to the subject a therapeutically effective amount of a compound of Formula (I) or a pharmaceutically acceptable salt thereof.

In any of the preceding methods, the method may further comprise obtaining or having obtained biological samples over a period of time from the subject and performing or having performed a bodily fluid test on the biological samples to determine whether the level of one or more biochemical markers are increasing or decreasing, and if the level of one or more biochemical markers are not trending in the desired direction then administering a greater dose of a compound of Formula (I) or a pharmaceutically acceptable salt thereof. For example, the ratio of HbF to HbS in a sample may be measured and a pronounced increase in the amount of HbF to HbS in a second sample relative to a first sample from a subject indicates that the dosage of a Formula (I) or a pharmaceutically acceptable salt thereof is a therapeutically effective dosage. Conversely, no change or no significant change in the amount of HbF to HbS in a second sample relative to a first sample from a subject may indicate that the dosage of a Formula (I) or a pharmaceutically acceptable salt thereof is not a therapeutically effective dosage and that the dosage may need to be increased.

In some embodiments, a compound of Formula (I) or a pharmaceutically acceptable salt thereof is administered to a subject in need thereof in an amount to decrease the level of one or more biomarker markers such as CRP or ROS.

The period between collection of biological samples may be 1 week, 2 weeks, 3 weeks, 4 weeks, 2 months, 3 months, 6 months, 9 months, or 12 months and the compound of Formula (I) or a pharmaceutically acceptable salt thereof may be administered during this period.

### A. Identification of Genetic Alterations

In some embodiments, the subject is selected for treatment based on identification of one or more genetic alterations in one or more alleles of the human beta-globin gene or expression control sequence thereof. Genetic alterations indicative of sickle cell disease, beta-thalassemia, or related disorders include the exemplary mutations discussed above, or other mutations that lead to a reduction in the synthesis, structure, or function of human beta-globin protein.

Methods of selecting a subject having one or more genetic alterations in one or more alleles of the beta-globin gene or expression control sequences thereof include the steps of obtaining a biological sample and detecting the presence or absence one or more genetic alterations. In an embodiment, the biological sample obtained contains nucleic acid from the subject and the step of detecting detects the presence or absence one or more genetic alterations in one or more alleles of the beta-globin gene or expression control sequences thereof in the biological sample. Any biological sample that contains the DNA of the subject to be diagnosed can be employed, including tissue samples and blood samples, with nucleated blood cells being a particularly convenient source. The DNA may be isolated from the biological sample prior to testing the DNA for the presence or absence of the genetic alterations.

The detecting step can include determining whether the subject is heterozygous or homozygous for a genetic alteration. The step of detecting the presence or absence of the genetic alteration can include the step of detecting the presence or absence of the alteration in both chromosomes of the subject (i.e., detecting the presence or absence of one or two alleles containing the marker or functional polymorphism). More than one copy of a genetic alterations (i.e., subjects homozygous for the genetic marker) can indicate a greater risk of developing sickle cell disease, beta-thalassemia, or related disorder. In some embodiments, the subject is heterozygous for two or more genetic alterations in the beta-globin gene (also referred to herein as double heterozygotes, triple heterozygotes, etc.). One copy of two or more genetic alterations in the beta-globin gene can indicate a greater risk of developing sickle cell disease, beta-thalassemia, or related disorder.

The process of determining the genetic sequence of human beta-globin gene is referred to as genotyping. In some embodiments, the human beta-globin gene is sequenced. Methods for amplifying DNA fragments and sequencing them are well known in the art. For example, automated sequencing procedures that can be utilized to sequence the beta-globin gene, include, but not limited to, sequencing by mass spectrometry single-molecule real-time sequencing, ion semiconductor (ion torrent sequencing), pyrosequencing (454), sequencing by synthesis, sequencing by ligation, chain termination (Sanger sequencing).

In some embodiments, the genotype of the subject is determined by identifying the presence of one or more single nucleotide polymorphisms (SNP) associated with sickle cell disease, beta-thalassemia, or a related disorder. Methods for

SNP genotyping are generally known in the art. SNP genotyping can include the steps of collecting a biological sample from a subject (e.g., sample of tissues, cells, fluids, secretions, etc.), isolating genomic DNA from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The neighboring sequence can be used to design SNP detection reagents such as oligonucleotide probes and primers. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Other suitable methods for detecting polymorphisms include methods in which protection from cleavage agents is used to detect mismatched bases in RNA/RNA or RNA/DNA duplexes, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis (DGGE). Sequence variations at specific locations can also be assessed by nuclease protection assays such as Rnase and S1 protection or chemical cleavage methods.

Another method for genotyping SNPs is the use of two oligonucleotide probes in an oligonucleotide ligation assay (OLA). Other methods that can be used to genotype the SNPs include single-strand conformational polymorphism (SSCP).

### B. Identification of Biochemical and Morphological Alterations

In some embodiments, subjects are selected for treatment based on identification of biochemical or morphological alterations or abnormalities in hemoglobin, or hemoglobin synthesizing cells such as hematopoietic stem cells, erythrocyte progenitor cells, erythrocytes, macrophage, retinal pigment epithelial cells, alveolar type II (ATII) cells, and others. The methods typically include identifying one or more biochemical or morphological alterations that is/are associated with a genetic alteration in the human beta-globin gene, or otherwise diagnostic of sickle cell disease, a beta-thalassemia, or a related disorder. Methods of diagnosing sickle cell disease, beta-thalassemia, or a related disorder according to biochemical or morphological alterations in the hemoglobin or hemoglobin synthesizing cells are known in the art, and include but are not limited to, analysis of erythrocyte morphology, osmotic fragility, hemoglobin composition, globin synthesis rates, and red blood cell indices.

In some embodiments, the method includes first testing a subject's blood for HbS, and selecting the subject for treatment if HbS is present. Methods for testing a subject's blood for the presence of HbS include solubility tests (e.g., SICKLEDEX) and sickling test. With the SICKLEDEX test, if HbS is present in a sample, it becomes insoluble and forms a cloudy suspension. Other hemoglobins are more soluble and will form a transparent solution. A sickling test can be used to determine if a red blood cell changes into a sickle shape after a blood sample is mixed with a reducing agent and identifying morphological changes to shape of red blood cells (i.e., “sickling”) by microscopy. Shape change of red blood cells may also be analyzed for shape change using a flow cytometer such as the Amnis ImageStreamX Mark II Imaging Flow Cytometer (MilliporeSigma). Shape change of red blood cells may be quantitated using a software program such as IDEAS application software (MilliporeSigma) using a modified protocol as described in “Imaging flow cytometry for automated detection of hypoxia-induced erythrocyte shape change in sickle cell disease.” van Beers E J, et al. Am J Hematol. 2014; 89 (6): 598-603; or as described in “Sickle Cell Imaging Flow Cytometry Assay (SIFCA).” Fertrin K Y, et al. Methods Mol Biol. 2016; 1389:279-292.

Other suitable tests include, hemoglobin electrophoresis, which employs gel electrophoretic techniques to separate out the various types of hemoglobin from a blood sample obtained from the subject. The test can detect abnormal levels of HbS, as well as other abnormal hemoglobins, such as hemoglobin C. It can also be used to determine whether there is a deficiency of any normal form of hemoglobin, as in various thalassemias. Alternatives to electrophoretic techniques include isoelectric focusing and chromatographic techniques. Other tests that can be used to select a subject for treatment with the compositions and methods disclosed herein include tests typically employed as part of a hemoglobinopathy screen, for example, a complete blood count (CBC) or iron study (ferritin). For example, a blood count can be used to detect anemia, and a blood smear and be used to identify sickled cells.

### EXAMPLES

### General Procedure A: Ipso Substitution of 6-chloro-5-nitro-nicotinic acid methyl ester

To a DMF or THF solution of a 6-chloro-5-nitro-nicotinic acid methyl ester is added 2 M methylamine in THF and the reaction mixture stirred at room temperature for 16 h. The resulting mixture is poured into water to precipitate the product. The precipitate may be filtered and dried to give the product, which may not be purified further before use in the next step.

### General Procedure B: Reduction of Nitro Group to Amine

10% Pd/C is added to a solution of the nitro compound in methanol. The resulting mixture is stirred at room temperature under a H₂ atmosphere for 16 h. The contents may then be filtered through a pad of Celite or silica gel and the solid washed with portions of methanol. The filtrate and washings are combined and evaporated to afford the corresponding diamine, which may not be purified further before use in the next step.

### General Procedure C: Thiourea Formation and its Conversion to 2-amino-imidazopyridine

1,1′-Thiocarbonylimidazole is added to a solution of an amine with triethylamine (1 eq.) in acetonitrile (10 mL). The reaction mixture is stirred at room temperature (1-24 h). The solvent is then evaporated, and the product suspended in acetonitrile. The solvent is then evaporated to produce the product as a precipitate. The precipitate is filtered and washed with acetonitrile and dried. The product may be used directly in the next step without further purification.

To the product obtained immediately above is added EDAC at room temperature followed by a substituted diaminopyridine, and the reaction mixture is stirred at 90° C. for 16 h. The reaction mixture is then cooled to room temperature, poured into cold water, and the solid collected by filtration. The crude product thus obtained may be purified by trituration with methanol.

### General Procedure D: Hydrolysis of Ester

A solution of NaOH in water is added to a solution of an ester in 1:1 THF/MeOH, and the resulting mixture is stirred at 60° C. for 16 h. After completion of the reaction, the mixture is concentrated under vacuum. The pH of the resulting suspension may be adjusted by the dropwise addition of 6 N HCl to PH ˜3, and the precipitate collected by filtration, washed with water and dried under vacuum. The desired carboxylic acid may be used without purification.

### General Procedure E: Amide Formation Using HBTU as Coupling Reagent

To a solution of a carboxylic acid in dry DMF is added DIEA followed by HBTU, and the reaction mixture is stirred at room temperature for 30 min. An appropriate amine is then added, and the reaction stirred at room temperature for 16 h. The contents may be diluted with ice-water, and the product precipitated. The product may be isolated after filtration either with subsequent washings with water and DCM/methanol or through silica gel chromatography using hexanes/ethyl acetate (from 80:20 to 60:40) as an eluent system.

### Compound 473

### N-[2-(2-Hydroxyethoxy)ethyl]-3-methyl-2-[[6-(trifluoromethyl)-1,3-benzothiazol-2-yl]amino]imidazo[4,5-b]pyridine-6-carboxamide

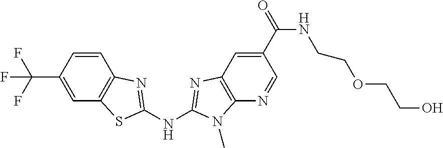 [SMILES: Cn1c(Nc2nc3ccc(C(F)(F)F)cc3s2)nc2cc(C(=O)NCCOCCO)cnc21]

6-Methylamino-5-nitro-nicotinic acid methyl ester (5.0 g) was prepared by following General Procedure A starting from 6-chloro-5-nitro-nicotinic acid methyl ester (5.0 g) and methylamine (33% in EtOH, 24 mL) in THF (150 mL). The crude product was used in the next step without further purification.

5-Amino-6-methylamino-nicotinic acid methyl ester (4.8 g) was prepared by following General Procedure B starting from 6-methylamino-5-nitro-nicotinic acid methyl ester (5.0 g) and Pd/C (20% by weight, 1.0 g) in methanol: THF (1:1, 50 mL). The crude product was used in the next step without further purification.

Methyl 3-methyl-2-[[6-(trifluoromethyl)-1,3-benzothiazol-2-yl]amino]imidazo[4,5-b]pyridine-6-carboxylate (5.0 g) was prepared by following General Procedure C starting from 6-(trifluoromethyl)-1,3-benzothiazol-2-amine (5.0 g), 5-amino-6-methylamino-nicotinic acid methyl ester (5.0 g), 1,l′-thiocarbonyl-diimidazole (5.0 g), and EDAC (4.5 g). The crude product was used in next step without further purification.

3-Methyl-2-[[6-(trifluoromethyl)-1,3-benzothiazol-2-yl]amino]imidazo[4,5-b]pyridine-6-carboxylic acid (4.2 g) was prepared by following General Procedure D starting from methyl 3-methyl-2-[[6-(trifluoromethyl)-1,3-benzothiazol-2-yl]amino]imidazo[4,5-b]pyridine-6-carboxylate (5.0 g) and NaOH (2N, 25 mL) in methanol: THF (2:1, 50 mL). The crude product was used in next step without further purification.

N-[2-(2-Hydroxyethoxy)ethyl]-3-methyl-2-[[6-(trifluoromethyl)-1,3-benzothiazol-2-yl]amino]imidazo[4,5-b]pyridine-6-carboxamide (40 mg) was prepared by following General Procedure E starting from 3-methyl-2-[[6-(trifluoromethyl)-1,3-benzothiazol-2-yl]amino]imidazo[4,5-b]pyridine-6-carboxylic acid (100 mg), 2-(2-aminoethoxy) ethanol (100 mg), HBTU (200 mg) and DIEA (0.2 mL) in DMF (2.0 mL). LC/MS: m/z 481.7. ¹H NMR (DMSO-d₆, 400 MHz): δ 8.67-8.59 (m, 2H), 8.29-8.23 (d, 2H), 7.71-7.69 (d, 1H), 4.61 (s, 1H), 3.68 (br s, 3H), 3.57-3.44 (m, 8H), 3.32 (br s, 2H).

### Compound 474

### 1-Ethyl-N-[2-(2-hydroxyethoxy)ethyl]-2-[[5-(trifluoromethoxy)-1,3-benzothiazol-2-yl]amino] benzimidazole-5-carboxamide

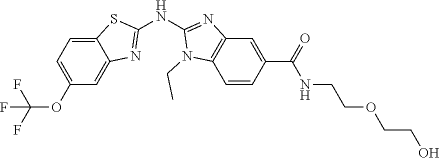 [SMILES: CCn1c(Nc2nc3cc(OC(F)(F)F)ccc3s2)nc2cc(C(=O)NCCOCCO)ccc21]

1-Ethyl-N-[2-(2-hydroxyethoxy)ethyl]-2-[[5-(trifluoromethoxy)-1,3-benzothiazol-2-yl]amino] benzimidazole-5-carboxamide (40 mg) was prepared by following General Procedure E starting from 3-ethyl-2-[[6-(trifluoromethoxy)-1,3-benzothiazol-2-yl]amino]imidazo[4,5-b]pyridine-6-carboxylic acid (100 mg) (See WO '018), 2-(2-aminoethoxy) ethanol (100 mg), HBTU (200 mg) and DIEA (0.2 mL) in DMF (2.0 mL). LC/MS: m/z 510.7.

### General Assay Methods

### Western Blot Assay

About 20-50 ug of protein is separated by SDS-polyacrylamide gel electrophoresis, and transferred to nitrocellulose membranes. Membranes are blocked in 5% dry milk containing TBS-T for 30 minutes followed by one hour and incubated with HbF or actin antibodies. After several washes, membranes are incubated with 1:10000 diluted HRP-conjugated secondary antibody (Thermo Scientific), developed with ECL Prime reagent (GE Healthcare Biosciences). Images may be captured on a Bio-Rad Chemi-Doc MP Imaging System and protein bands quantified by densitometry.

### Flow Cytometry Assay

About 5×105 cells are harvested after treatment with compound, washed twice with ice cold phosphate buffered saline and resuspended in 4% paraformaldehyde for 40 minutes at 37° C. Fixed cells are permeabilized with ice-cold acetone/methanol (4:1) and washed with phosphate buffered saline followed by incubation with FITC-conjugated anti-HbF antibody (1:1000, Abcam) for 20 minutes. The labeled cells may be analyzed using a Becton Dickerson LSR-II flow cytometer (BD Bioscience, San Jose, CA, USA) and FlowJo v0.9 software.

### Example 1

KU812, a human leukemic cell line that expresses the fetal gamma-globin and adult beta-globin genes, was used as a system for screening. KU812 cells have comparable globin gene response patterns as primary erythroid cells after treatments with potential HbF inducers. (See Zein S, Lou R F, Sivanand S, Ramakrishnan V, Mackie A, Li W, Pace BS. KU812 Cell Line: model for identifying fetal hemoglobin inducing drugs. Exp Biol Med (Maywood) 235:1385-94, 2010.) KU812 cells were grown in Iscove's Modified Dulbecco Media (IMDM) and 10% fetal bovine serum until in log phase growth.

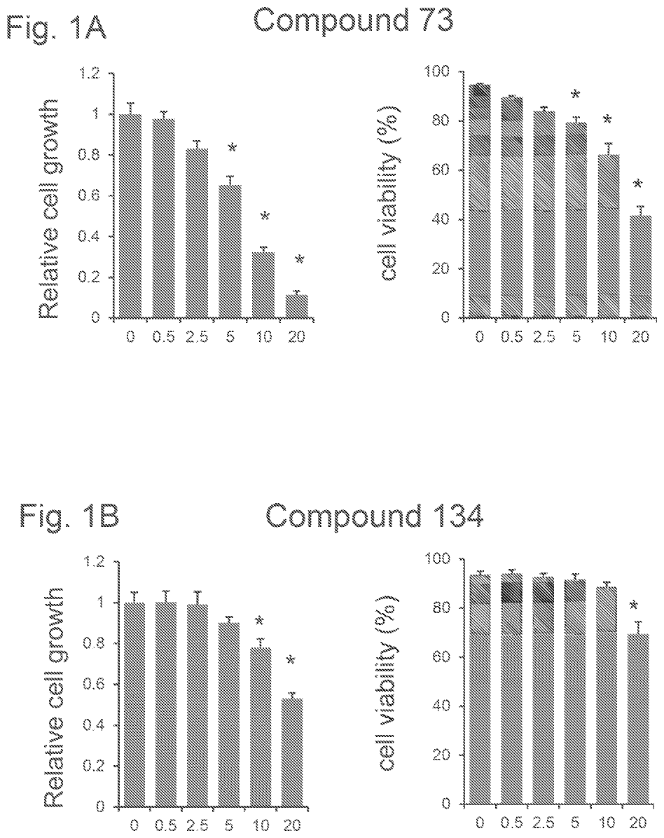

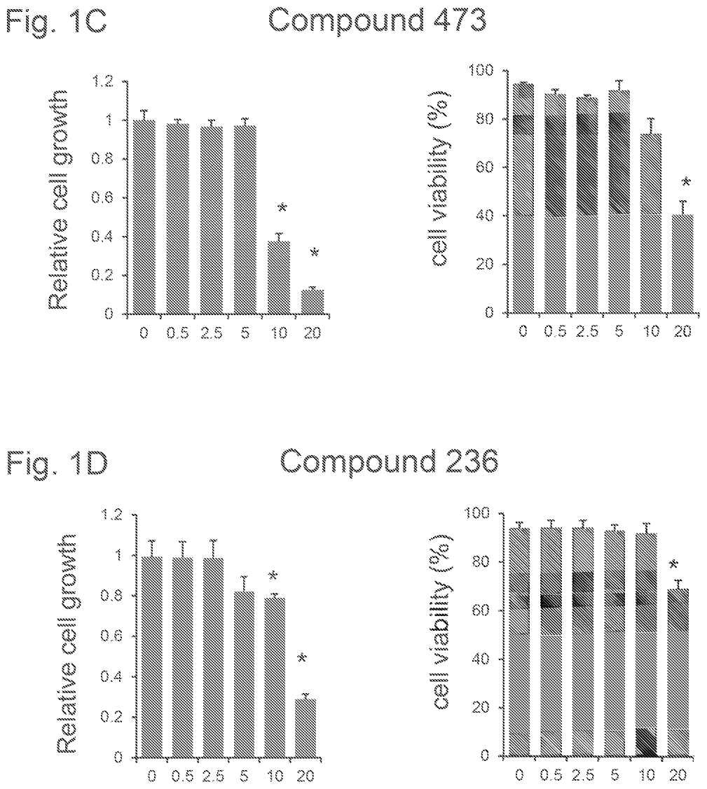

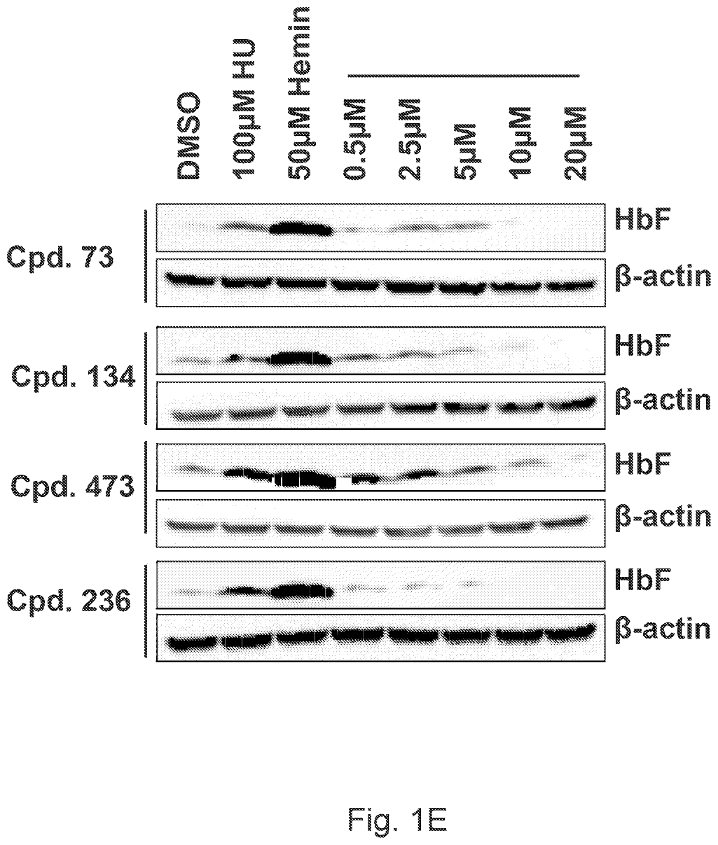

KU812 cells in log growth phase were treated with compounds 73, 134, 473 and 236 (See Table A) at a doses of 0.5, 2.5, 5.0 and 20 μM for 48 hours. At harvest, cell counts and viability were measured by 0.4% Trypan blue exclusion. See FIGS. 1A-1D. Compounds 134 and 473 had minimal effects on cell growth rates and viability remained >90% at the widest range of drug concentrations (See FIGS. 1B and 1C, respectively).

Western blot analysis was also conducted to determine level of induction of HbF in KU812 cells by Compounds 73, 134, 473, and 236. Hydroxyurea and hemin were used as positive controls, and ß-actin was used as a protein loading control. Compounds 73, 134 and 473 each showed HbF induction, and compounds 134 and 473 increased HbF at concentrations between 0.5 to 5 PM (FIG. 1E). Compound 236 did not show significant induction of HbF by Western blot analysis at the tested concentrations.

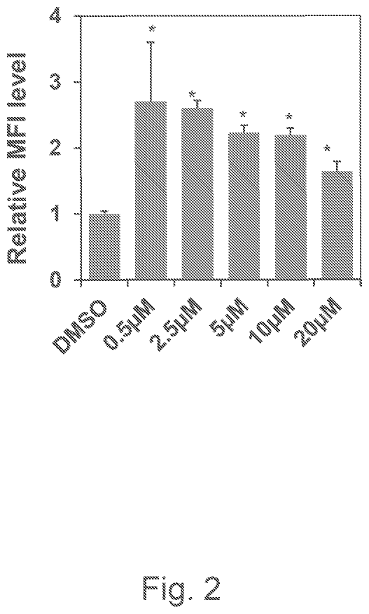

Flow cytometry was conducted for KU812 cells following treatment with Compounds 473 and 236 at various concentrations (0.5, 2.5, 5, 10, and 20 μM). An increase in the number of HbF positive cells (F-cells) and increased mean fluorescence intensity (MFI) was observed for Compound 473 (FIG. 2). By contrast, Compound 236 did not significantly increase F-cells at these concentrations, but some increase in MFI was observed (data not shown). Compounds 73 and 134 were not analyzed by flow cytometry.

### Example 2

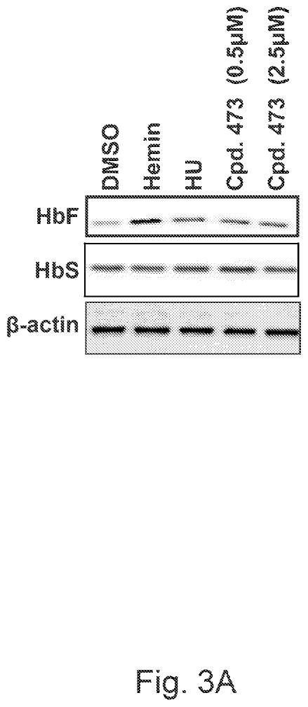

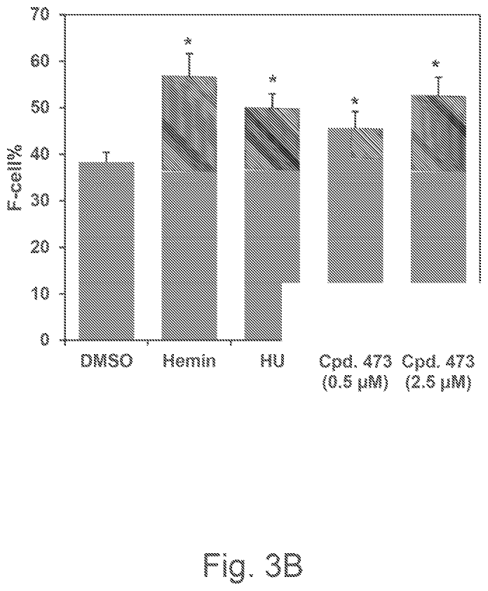

To test compounds under conditions more closely modeling physiological conditions for one with a SCD, sickle erythroid progenitor cells were cultured for 10 days and then treated with Compound 473 for 48 hours at concentrations of 0.5 μM and 2.5 μM. Treated cells were analyzed by western blot for levels of expression of HbF, HbS, and ß-actin relative to cells treated with DMSO, hemin, or HU. The same treated cells were also analyzed by flow cytometry for y-globin gene expression relative to cells treated with DMSO, hemin, or HU. Compound 473 (0.5 μM and 2.5 μM) induced γ-globin gene expression by 1.6 and 1.9 fold, respectively, without affecting HbS protein levels. See FIG. 3A. Increased F-cell levels were observed by flow cytometry. See FIG. 3B.

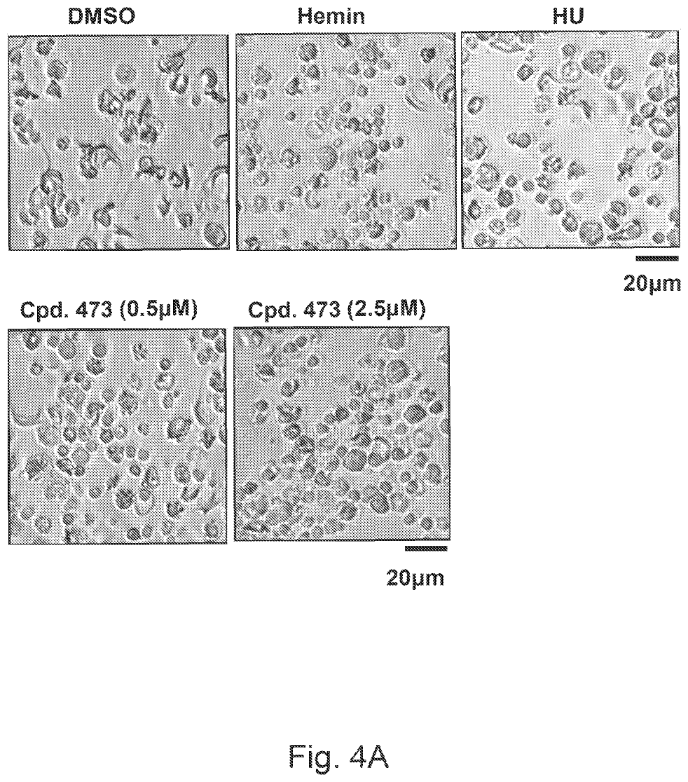

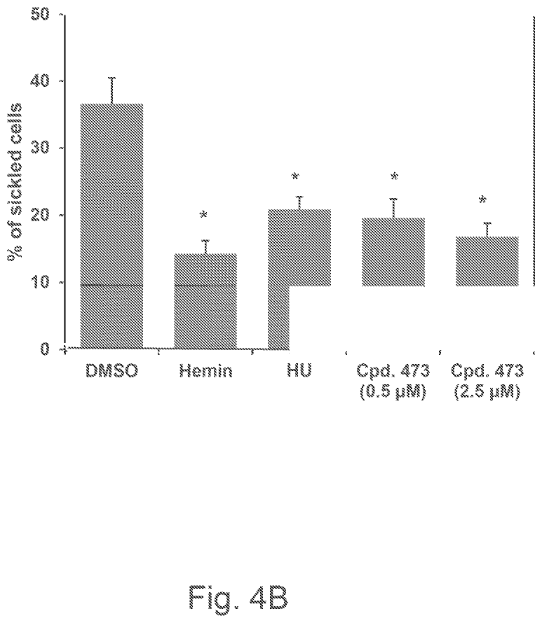

Anti-sickling activity was observed in treated cells under hypoxia conditions. As described above, sickle erythroid progenitor cells were cultured for 10 days and then treated with Compound 473 for 48 hours at concentrations of 0.5 μM and 2.5 μM or with hemin (about 50 μM) or with HU (about 100 μM). Treated cells were then subjected to hypoxia conditions (1% O₂ and 5% CO₂). Cells treated with Compound 473 at concentrations of 0.5 μM and 2.5 μM significantly decreased the percent of sickled cells compared to DMSO control. See FIGS. 4A and 4B.

What is claimed is:

1. A method of treating a sickle cell disorder in a subject comprising administering to the subject with the sickle cell disorder a therapeutically effective amount of a compound that is:
1-methyl-2-(6-trifluoromethyl-benzothiazol-2-ylamino)-1H-benzimidazole-5-carboxylic acid dimethylcarbamoylmethyl-amide, or
1-methyl-2-(6-trifluoromethyl-benzothiazol-2-ylamino)-1H-benzoimidazole-5-carboxylic acid [2-(2-hydroxy-ethoxy)-ethyl]-amide, or
N—[2-(2-hydroxyethoxy)ethyl]-3-methyl-2-[[6-(trifluoromethyl)-1,3-benzothiazol-2-yl]amino]imidazo[4,5-b]pyridine-6-carboxamide,
or a pharmaceutically acceptable salt thereof.

2. The method of claim 1, wherein the compound is 1-methyl-2-(6-trifluoromethyl-benzothiazol-2-ylamino)-1H-benzimidazole-5-carboxylic acid dimethylcarbamoylmethyl-amide or a pharmaceutically acceptable salt thereof.

3. The method of claim 1, wherein the compound is 1-methyl-2-(6-trifluoromethyl-benzothiazol-2-ylamino)-H-benzoimidazole-5-carboxylic acid [2-(2-hydroxy-ethoxy)-ethyl]-amide or a pharmaceutically acceptable salt thereof.

4. The method of claim 1, wherein the compound is N-[2-(2-hydroxyethoxy)ethyl]-3-methyl-2-[[6-(trifluoromethyl)-1,3-benzothiazol-2-yl]amino]imidazo[4,5-b]pyridine-6-carboxamide or a pharmaceutically acceptable salt thereof.

5. The method of claim 1, wherein treating the sickle cell disorder comprises increasing expression of fetal hemoglobin expression (HbF) in the subject.

6. The method of claim 1, wherein treating the sickle cell disorder comprises reducing blood cell sickling in response to reduced air pressure, reduced barometric pressure, reduced partial pressure of oxygen, or hypoxia.

7. The method of claim 1, wherein treating the sickle cell disorder comprises
reducing the incidences or rate of painful crises,
reducing incidences or rate of painful crises requiring hospitalization,
reducing incidences of chest syndrome,
reducing the number of transfusion events, or
reducing the number of units of blood transfused per event.

8. The method of claim 1, wherein treating the sickle cell disorder comprises treating hemolytic anemia, a vaso-occlusive crisis, or multiple organ damage from microinfarcts.

9. The method of claim 1, wherein the subject exhibits one or more of the clinical symptoms of sickle cell disease, beta-thalassemia, or a related disorder caused by the sickle cell disorder.

10. The method of claim 1, wherein the subject exhibits a genetic or biochemical indicator of sickle cell disease, beta-thalassemia, or a related disorder caused by the sickle cell disorder.

11. The method of claim 1, wherein the subject has sickle cell disease, beta-thalassemia, or a related disorder caused by the sickle cell disorder.

12. The method of claim 1, wherein the compound or the pharmaceutically acceptable salt thereof is administered in combination with another active compound selected from the group consisting of hydroxyurea, dimethyl fumarate, monomethyl fumarate, and bardoxolone methyl.

13. The method of claim 1, wherein from 10 mg/day to 100 mg/day of the compound is administered.

14. A method of treating a sickle cell disorder in a subject comprising orally administering to the subject with the sickle cell disorder a pharmaceutical composition comprising a compound that is:
1-methyl-2-(6-trifluoromethyl-benzothiazol-2-ylamino)-1H-benzimidazole-5-carboxylic acid dimethylcarbamoylmethyl-amide, or
1-methyl-2-(6-trifluoromethyl-benzothiazol-2-ylamino)-1H-benzoimidazole-5-carboxylic acid [2-(2-hydroxy-ethoxy)-ethyl]-amide, or
N—[2-(2-hydroxyethoxy)ethyl]-3-methyl-2-[[6-(trifluoromethyl)-1,3-benzothiazol-2-yl]amino]imidazo[4,5-b]pyridine-6-carboxamide,
or a pharmaceutically acceptable salt thereof.

15. The method of claim 14, wherein the pharmaceutical composition is formulated as a capsule or tablet for oral administration.
